SEQUENCE LISTING (124 sequences)

SEQ ID NO: 1 - protein, 341 aa, Streptococcus pyogenes
MRKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 219 aa, Synthetic construct
EVQLVETGGGLVQPGKSLKLTCATSGFTFSNAWMHWVRQSPEKQLEWVSQIRAKSNNYATYYAESVKGRFTISRDDSKRNVYLQMNSLKEEDTAIYYCTTGSGDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - DNA, 657 bp, Synthetic construct
  source 1..657: mol_type = other DNA
gaagtacaactggtagaaacaggaggggggctggtccagcctggcaaaagcctgaaactgacgtgcgctacgagcggctttacattttcaaacgcttggatgcactgggttaggcagtcccctgagaagcaattggaatgggtaagccagatacgagctaagtcaaacaactacgcgacgtattatgcagagtctgtaaaaggaaggttcacgataagtcgggatgactctaaaaggaacgtgtacctgcaaatgaacagcctcaaggaagaagatacagccatttactattgtaccacgggctctggagattactggggtcaaggagtcatggtcactgtttcaagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 4 - protein, 5 aa, Synthetic construct
NAWMH

SEQ ID NO: 5 - protein, 19 aa, Synthetic construct
QIRAKSNNYATYYAESVKG

SEQ ID NO: 6 - protein, 5 aa, Synthetic construct
GSGDY

SEQ ID NO: 7 - protein, 221 aa, Synthetic construct
DVVLTQTPPTLLATIGQSVSISCRSSQSLLHRSGNTYLNWLLQRTGQSPQPLIYLVSKLESGVPNRFSGSGSGTDFTLKISGVEAEDLGIYYCMQFTHYPYTFGAGTKLELKRARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 663 bp, Synthetic construct
  source 1..663: mol_type = other DNA
gacgttgttctgactcaaacaccaccaacactccttgcaaccatcggtcaatctgtttcaatatcatgcagaagttcccagtccttgttgcaccgaagtggtaacacgtacttgaactggctcctccaacggacaggacaaagccctcagccgctcatttatttggtttccaagctggagtccggtgtaccgaacagattctcaggttccggtagtggcacagacttcaccttgaaaattagtggcgtggaggcggaagacctgggaatttattattgtatgcaattcactcattacccgtacacgttcggcgcaggcaccaagttggagttgaaacgggcacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 9 - protein, 16 aa, Synthetic construct
RSSQSLLHRSGNTYLN

SEQ ID NO: 10 - protein, 7 aa, Synthetic construct
LVSKLES

SEQ ID NO: 11 - protein, 9 aa, Synthetic construct
MQFTHYPYT

SEQ ID NO: 12 - protein, 541 aa, Synthetic construct
EVQLVETGGGLVQPGKSLKLTCATSGFTFSNAWMHWVRQSPEKQLEWVSQIRAKSNNYATYYAESVKGRFTISRDDSKRNVYLQMNSLKEEDTAIYYCTTGSGDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 13 - DNA, 1623 bp, Synthetic construct
  source 1..1623: mol_type = other DNA
gaagtacaactggtagaaacaggaggggggctggtccagcctggcaaaagcctgaaactgacgtgcgctacgagcggctttacattttcaaacgcttggatgcactgggttaggcagtcccctgagaagcaattggaatgggtaagccagatacgagctaagtcaaacaactacgcgacgtattatgcagagtctgtaaaaggaaggttcacgataagtcgggatgactctaaaaggaacgtgtacctgcaaatgaacagcctcaaggaagaagatacagccatttactattgtaccacgggctctggagattactggggtcaaggagtcatggtcactgtttcaagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 14 - protein, 224 aa, Synthetic construct
EVQLLESGPGLVKPSETLSLTCTVSGSSLSSAYGWNWIRQPPGKGLEWIGSIGGSRDNTNYNPSLKRRVTISKDTSKNQFSLKLKSVTAADTAVYYCAQRGAYGYSYFDYWGQGVLVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - DNA, 672 bp, Synthetic construct
  source 1..672: mol_type = other DNA
gaggtacaactcctggaatctggaccagggcttgtaaagccatccgaaaccctgtcacttacttgtactgtctctggctcatctttgtcaagcgcctacggttggaattggatacgacaacccccaggaaagggccttgaatggatagggagcatcggaggttcccgcgacaacacgaattacaatccgagtttgaaacgccgggttacaatatccaaggatacgagcaagaaccagttttctctcaaactgaaatcagtaacagcagctgacactgccgtatattactgtgctcaacgcggcgcctatggatatagttacttcgactactggggccagggcgtcttggtggcagtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 16 - protein, 6 aa, Synthetic construct
SAYGWN

SEQ ID NO: 17 - protein, 17 aa, Synthetic construct
SIGGSRDNTNYNPSLKR

SEQ ID NO: 18 - protein, 11 aa, Synthetic construct
RGAYGYSYFDY

SEQ ID NO: 19 - protein, 215 aa, Synthetic construct
ELTLTQSPATLSLSPGETATLSCRASQTVGRNLAWYQQRPGQAPNLLVHSAYFRATGIPDRFSGSGSGTDFTLTISSLEPEDAGVYHCQQYNDLLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 645 bp, Synthetic construct
  source 1..645: mol_type = other DNA
gagttgacactgacgcagtcaccggcgactcttagtctctcccctggcgaaaccgcgactctttcttgtcgcgcatcccaaacggttggacgaaatttggcgtggtatcaacagcgccccggacaggccccgaatctgttggtgcattccgcatatttcagggcgactggtattcctgaccgcttttccggatcaggttccggaacggatttcacactcactattagttccttggaaccagaagatgcgggagtttaccattgccaacaatataacgatctgctcccacttactttcggtgggggcaccaaggtagagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 21 - protein, 11 aa, Synthetic construct
RASQTVGRNLA

SEQ ID NO: 22 - protein, 7 aa, Synthetic construct
SAYFRAT

SEQ ID NO: 23 - protein, 10 aa, Synthetic construct
QQYNDLLPLT

SEQ ID NO: 24 - protein, 546 aa, Synthetic construct
EVQLLESGPGLVKPSETLSLTCTVSGSSLSSAYGWNWIRQPPGKGLEWIGSIGGSRDNTNYNPSLKRRVTISKDTSKNQFSLKLKSVTAADTAVYYCAQRGAYGYSYFDYWGQGVLVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 25 - DNA, 1638 bp, Synthetic construct
  source 1..1638: mol_type = other DNA
gaggtacaactcctggaatctggaccagggcttgtaaagccatccgaaaccctgtcacttacttgtactgtctctggctcatctttgtcaagcgcctacggttggaattggatacgacaacccccaggaaagggccttgaatggatagggagcatcggaggttcccgcgacaacacgaattacaatccgagtttgaaacgccgggttacaatatccaaggatacgagcaagaaccagttttctctcaaactgaaatcagtaacagcagctgacactgccgtatattactgtgctcaacgcggcgcctatggatatagttacttcgactactggggccagggcgtcttggtggcagtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 26 - protein, 231 aa, Synthetic construct
EVQLLESGPGLLKPSETLSLTCAVSGAPISNYWWSWIRQSPGKGLEWIGEIDGSIYTTYYNPSLKSRVAISKDTSKNRLSLKLTSVTAADTAVYYCAREGQNPLVPTYGSTGFGLDFWGHGLAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - DNA, 672 bp, Synthetic construct; identical to SEQ ID NO: 15
  source 1..672: mol_type = other DNA

SEQ ID NO: 28 - protein, 5 aa, Synthetic construct
NYWWS

SEQ ID NO: 29 - protein, 17 aa, Synthetic construct
EIDGSIYTTYYNPSLKS

SEQ ID NO: 30 - protein, 19 aa, Synthetic construct
EGQNPLVPTYGSTGFGLDF

SEQ ID NO: 31 - protein, 213 aa, Synthetic construct
AELTQSPSSLSASVGDRVTITCQASQGISSWLAWYQQKPGKAPKLLIYKASSLQSGVPSRFSGSGSGTDFTLTISSLQSEDFATYYCQQYSSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 639 bp, Synthetic construct
  source 1..639: mol_type = other DNA
gctgagttgacgcagagtcctagtagcctctctgcgtctgtaggagatcgggtcaccatcacatgccaagcatcacaaggtatctcctcatggcttgcttggtaccaacagaaaccaggtaaagcacctaagctgcttatatacaaagcctctagcttgcagagtggcgtcccgtcccgctttagcggctccggctcagggacggactttacattgacgatctcttcacttcagtctgaagattttgcaacctactattgccagcaatactcctcctctccgagaactttcggtcagggaacaaaggttgaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 33 - protein, 11 aa, Synthetic construct
QASQGISSWLA

SEQ ID NO: 34 - protein, 7 aa, Synthetic construct
KASSLQS

SEQ ID NO: 35 - protein, 9 aa, Synthetic construct
QQYSSSPRT

SEQ ID NO: 36 - protein, 553 aa, Synthetic construct
EVQLLESGPGLLKPSETLSLTCAVSGAPISNYWWSWIRQSPGKGLEWIGEIDGSIYTTYYNPSLKSRVAISKDTSKNRLSLKLTSVTAADTAVYYCAREGQNPLVPTYGSTGFGLDFWGHGLAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 37 - DNA, 1659 bp, Synthetic construct
  source 1..1659: mol_type = other DNA
gaagtgcaattgctggagagcgggccgggccttctcaaaccaagtgaaactctgtcccttacctgcgcggtctcaggggcgcccataagcaattattggtggtcctggatacgccagagtcctggtaaaggcttggagtggattggggaaattgatggctctatctatacaacatattacaacccgtcattgaagagccgcgtggctatttcaaaagacacatccaagaaccgactttctctgaagctcacatctgtaactgccgccgatacggcagtatattactgcgccagggaaggacagaatcctcttgtaccaacttatggcagcacaggattcggactcgacttttggggccatggattggcggttacggtgagttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 38 - protein, 223 aa, Synthetic construct
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - DNA, 669 bp, Synthetic construct
  source 1..669: mol_type = other DNA
caagtaaaattgcaggagagtggtgggggactcgtacagccagggggttcccttaaattgtcatgcgttgcctctggttttaccttcagagatcattggatgaactgggtcagacaagcgcctggtaaaactatggaatggattggggacatacgcccagatggatctgacactaactatgcaccaagtgttcgcaatagatttacaatatctagagataatgcccgatctatcttgtatttgcagatgagcaacatgcgaagtgactatacggcaacgtactactgtgtaagagattcaccaactcgagcgggacttatggacgcatggggccagggtgcaagtgtgaccgtactcacggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 40 - protein, 5 aa, Synthetic construct
DHWMN

SEQ ID NO: 41 - protein, 17 aa, Synthetic construct
DIRPDGSDTNYAPSVRN

SEQ ID NO: 42 - protein, 11 aa, Synthetic construct
DSPTRAGLMDA

SEQ ID NO: 43 - protein, 213 aa, Synthetic construct
DIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 639 bp, Synthetic construct
  source 1..639: mol_type = other DNA
gacatacaaatgatacagtctccatctgtcctctctgcgagcgtcggagatcgcgtcaccttgaactgtaaggcgtctcagaatattaacaagtatttgaactggtatcagcagaagctcggtgaggcccctaaggtactcatatacaacactaataaccttcagacaggcattccaagccgatttagcggttccggaagtggaactgacttcacactgaccatctccagcctccaacccgaggattttgctacgtatttctgtttccagcattatacttggcctaccttcggcggcggtacgaaactggaactgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 45 - protein, 5 aa, Synthetic construct
DHWMN

SEQ ID NO: 46 - protein, 17 aa, Synthetic construct
DIRPDGSDTNYAPSVRN

SEQ ID NO: 47 - protein, 11 aa, Synthetic construct
DSPTRAGLMDA

SEQ ID NO: 48 - protein, 545 aa, Synthetic construct
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 49 - DNA, 1635 bp, Synthetic construct
  source 1..1635: mol_type = other DNA
caagtaaaattgcaggagagtggtgggggactcgtacagccagggggttcccttaaattgtcatgcgttgcctctggttttaccttcagagatcattggatgaactgggtcagacaagcgcctggtaaaactatggaatggattggggacatacgcccagatggatctgacactaactatgcaccaagtgttcgcaatagatttacaatatctagagataatgcccgatctatcttgtatttgcagatgagcaacatgcgaagtgactatacggcaacgtactactgtgtaagagattcaccaactcgagcgggacttatggacgcatggggccagggtgcaagtgtgaccgtactcacggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 50 - protein, 220 aa, Synthetic construct
EVQLQQSGPELVKPGASVKISCKVSGYSFTDYNVDWVKQSHRKTLEWIGYIYPDNGVTDYTQKFNTKATLTVDKSSSTAYMDLHSLTSEDSAVYYCARGRGWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - DNA, 660 bp, Synthetic construct
  source 1..660: mol_type = other DNA
gaggtgcagctccagcagtcaggtcccgagctggtaaagcccggagcctcagtgaagatctcttgtaaggtatctggatacagtttcacggattacaacgttgattgggttaagcagtctcacagaaagaccctggaatggataggctatatttatccagataatggtgttacagactatacacaaaaattcaacactaaggcgacactcacagtcgataaaagttcttccaccgcgtatatggacttgcactctctgacttcagaagattccgccgtctactactgtgccagagggaggggctggtactttgacgtgtggggcgcgggcactactgtcacagtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 52 - protein, 5 aa, Synthetic construct
DYNVD

SEQ ID NO: 53 - protein, 17 aa, Synthetic construct
YIYPDNGVTDYTQKFNT

SEQ ID NO: 54 - protein, 8 aa, Synthetic construct
GRGWYFDV

SEQ ID NO: 55 - protein, 218 aa, Synthetic construct
DIVLTQSPASLAVSLGQRATISCRASESVDNYGISFMNWLQQKPGQPPKLLIYAASNQVSGVPARFSGSGSGTDFSLNIHPMEEDDTAMYFCQQSKEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - DNA, 654 bp, Synthetic construct
  source 1..654: mol_type = other DNA
gacattgttttgacccagagcccggccagcttggctgtgagtctgggccaacgcgctacaatctcctgtcgcgcgagtgaatcagtggataattatggtatttcattcatgaattggctgcaacagaaaccgggacaacctccaaagctccttatatatgcggcttccaatcaagtaagcggggtcccagcccggttttctggttccggctccggtactgactttagtttgaacattcacccaatggaagaagatgataccgccatgtacttctgccaacaatcaaaggaaattccttatacctttggcggcggcacgaagctggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 57 - protein, 15 aa, Synthetic construct
RASESVDNYGISFMN

SEQ ID NO: 58 - protein, 7 aa, Synthetic construct
AASNQVS

SEQ ID NO: 59 - protein, 9 aa, Synthetic construct
QQSKEIPYT

SEQ ID NO: 60 - protein, 542 aa, Synthetic construct
EVQLQQSGPELVKPGASVKISCKVSGYSFTDYNVDWVKQSHRKTLEWIGYIYPDNGVTDYTQKFNTKATLTVDKSSSTAYMDLHSLTSEDSAVYYCARGRGWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 61 - DNA, 1626 bp, Synthetic construct
  source 1..1626: mol_type = other DNA
gaggtgcagctccagcagtcaggtcccgagctggtaaagcccggagcctcagtgaagatctcttgtaaggtatctggatacagtttcacggattacaacgttgattgggttaagcagtctcacagaaagaccctggaatggataggctatatttatccagataatggtgttacagactatacacaaaaattcaacactaaggcgacactcacagtcgataaaagttcttccaccgcgtatatggacttgcactctctgacttcagaagattccgccgtctactactgtgccagagggaggggctggtactttgacgtgtggggcgcgggcactactgtcacagtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 62 - protein, 253 aa, Synthetic construct
QVQLVQSGAEVKKPGASVKVSCKASGYTFTNYGMNWVRQAPGQRLEWMGWLNTYTGESWYPDDFKGRVTITRDTSASTAYMELSSLRSEDTAVYYCARGDYGYDDPLDYWGQGTLVTVSSGAGGSGGGGSGGGGSGGGGSDIVMTQSPLSLPVTPGEPASISCRSSKSLLHTNQNTYLHWFLQKPGQSPQLLIYRMSVLASGVPDRFSGSGSGTDFTLKISRVEAEDVGVYYCMQHLEYPLTFGQGTKLEIKR

SEQ ID NO: 63 - protein, 271 aa, Synthetic construct
QVQLVQSGAEVKKPGASVKVSCKASGYTFTNYGMNWVRQAPGQRLEWMGWLNTYTGESWYPDDFKGRVTITRDTSASTAYMELSSLRSEDTAVYYCARGDYGYDDPLDYWGQGTLVTVSSGAGGSGGGGSGGGGSGGGGSDIVMTQSPLSLPVTPGEPASISCRSSKSLLHTNQNTYLHWFLQKPGQSPQLLIYRMSVLASGVPDRFSGSGSGTDFTLKISRVEAEDVGVYYCMQHLEYPLTFGQGTKLEIKRGSSSGLPETGGGHHHHHH

SEQ ID NO: 64 - protein, 265 aa, Synthetic construct
GGGSSGQVQLVQSGAEVKKPGASVKVSCKASGYTFTNYGMNWVRQAPGQRLEWMGWLNTYTGESWYPDDFKGRVTITRDTSASTAYMELSSLRSEDTAVYYCARGDYGYDDPLDYWGQGTLVTVSSGAGGSGGGGSGGGGSGGGGSDIVMTQSPLSLPVTPGEPASISCRSSKSLLHTNQNTYLHWFLQKPGQSPQLLIYRMSVLASGVPDRFSGSGSGTDFTLKISRVEAEDVGVYYCMQHLEYPLTFGQGTKLEIKRHHHHHH

SEQ ID NO: 65 - protein, 5 aa, Synthetic construct
NYGMN

SEQ ID NO: 66 - protein, 17 aa, Synthetic construct
WLNTYTGESWYPDDFKG

SEQ ID NO: 67 - protein, 11 aa, Synthetic construct
GDYGYDDPLDY

SEQ ID NO: 68 - protein, 16 aa, Synthetic construct
RSSKSLLHTNQNTYLH

SEQ ID NO: 69 - protein, 7 aa, Synthetic construct
RMSVLAS

SEQ ID NO: 70 - protein, 9 aa, Synthetic construct
MQHLEYPLT

SEQ ID NO: 71 - protein, 328 aa, Synthetic construct
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGSSSGLPETGGGHHHHHH

SEQ ID NO: 72 - protein, 321 aa, Synthetic construct
GGGSSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 73 - protein, 583 aa, Synthetic construct
QVQLVQSGAEVKKPGASVKVSCKASGYTFTNYGMNWVRQAPGQRLEWMGWLNTYTGESWYPDDFKGRVTITRDTSASTAYMELSSLRSEDTAVYYCARGDYGYDDPLDYWGQGTLVTVSSGAGGSGGGGSGGGGSGGGGSDIVMTQSPLSLPVTPGEPASISCRSSKSLLHTNQNTYLHWFLQKPGQSPQLLIYRMSVLASGVPDRFSGSGSGTDFTLKISRVEAEDVGVYYCMQHLEYPLTFGQGTKLEIKRGSSSGLPETGGGSSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 74 - protein, 584 aa, Synthetic construct
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGSSSGLPETGGGSSGQVQLVQSGAEVKKPGASVKVSCKASGYTFTNYGMNWVRQAPGQRLEWMGWLNTYTGESWYPDDFKGRVTITRDTSASTAYMELSSLRSEDTAVYYCARGDYGYDDPLDYWGQGTLVTVSSGAGGSGGGGSGGGGSGGGGSDIVMTQSPLSLPVTPGEPASISCRSSKSLLHTNQNTYLHWFLQKPGQSPQLLIYRMSVLASGVPDRFSGSGSGTDFTLKISRVEAEDVGVYYCMQHLEYPLTFGQGTKLEIKRHHHHHH

SEQ ID NO: 75 - protein, 225 aa, Synthetic construct
QVQLQQPGAVLVKPGASVKMSCTASGYTFTNYNMHWVKQTPGQGLEWIGGIYPGNGNTAYNQKFKGKATVTADKSSSAVHMQVSSLTSEDSAVYYCARRSGNSYSYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - DNA, 675 bp, Synthetic construct
  source 1..675: mol_type = other DNA
caggtgcaactgcagcagcctggggctgtgctggtgaagcctggggcctcagtgaagatgtcctgcacggcttctggctacacatttaccaattacaatatgcactgggtaaaacagacacctggacagggcctggaatggattggaggtatttatccaggaaatggtaatactgcctacaatcagaaattcaaaggcaaggccacagtgactgcagacaaatcctccagcgcagtccacatgcaggtcagcagcctgacatctgaggactctgcggtctattactgtgcacgacgctccggtaatagctactcctactggtacttcgatgtctggggcgcagggaccacggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 77 - protein, 5 aa, Synthetic construct
NYNMH

SEQ ID NO: 78 - protein, 17 aa, Synthetic construct
GIYPGNGNTAYNQKFKG

SEQ ID NO: 79 - protein, 13 aa, Synthetic construct
RSGNSYSYWYFDV

SEQ ID NO: 80 - protein, 213 aa, Synthetic construct
DIKMTQSPSSIYASLGERVTITCKASQDINNYLTWYLQKPGKSPKTLIYRANRLVDGVPSRFSGSGSGQDYSLTISSLEFEDMGIYYCLQYDELYTFGGGTKVELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 639 bp, Synthetic construct
  source 1..639: mol_type = other DNA
gatatcaagatgacccagtctccatcttccatatatgcatctcttggagagagagtcactatcacttgcaaggcgagtcaggacattaataactatttaacctggtacctgcagaaaccagggaaatctcctaagaccctgatctatcgtgcaaacagattggtagatggggtcccatcaaggttcagtggcagtggatctggacaagattattctctcaccatcagcagcctggagtttgaagatatgggaatttattactgtctacagtatgatgagttgtacacgttcggaggtggaaccaaggtcgaattgaggcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 82 - protein, 11 aa, Synthetic construct
KASQDINNYLT

SEQ ID NO: 83 - protein, 7 aa, Synthetic construct
RANRLVD

SEQ ID NO: 84 - protein, 8 aa, Synthetic construct
LQYDELYT

SEQ ID NO: 85 - protein, 547 aa, Synthetic construct
QVQLQQPGAVLVKPGASVKMSCTASGYTFTNYNMHWVKQTPGQGLEWIGGIYPGNGNTAYNQKFKGKATVTADKSSSAVHMQVSSLTSEDSAVYYCARRSGNSYSYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 86 - DNA, 1641 bp, Synthetic construct
  source 1..1641: mol_type = other DNA
caggtgcaactgcagcagcctggggctgtgctggtgaagcctggggcctcagtgaagatgtcctgcacggcttctggctacacatttaccaattacaatatgcactgggtaaaacagacacctggacagggcctggaatggattggaggtatttatccaggaaatggtaatactgcctacaatcagaaattcaaaggcaaggccacagtgactgcagacaaatcctccagcgcagtccacatgcaggtcagcagcctgacatctgaggactctgcggtctattactgtgcacgacgctccggtaatagctactcctactggtacttcgatgtctggggcgcagggaccacggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 87 - protein, 226 aa, Synthetic construct
QVQLQQSGAELMKPGASVKISCKATGYTFSHYWIEWVKQRPGHGLEWIGEILPVTGSTKYTEKFKDKATFTADTFSNTAYMQLTSLTSEDSAVYYCARDPHYSGSTYDAMDYWGQGTSVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - DNA, 678 bp, Synthetic construct
  source 1..678: mol_type = other DNA
caggtgcagcttcaacaaagcggagcagagctgatgaagccaggcgctagtgtgaagatctcatgcaaggccaccggctacacgttcagccactattggatcgagtgggtgaaacagaggcccggtcatggcctggagtggatcggcgagatcctgcccgtgaccggctccaccaagtacaccgagaagttcaaggacaaggctaccttcaccgccgacaccttcagcaataccgcctacatgcaactgacaagcctgaccagcgaggacagcgccgtgtactactgtgccagggaccctcactacagcggcagcacctacgacgccatggactactggggccagggcaccagcgtaacagtgagcgccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 89 - protein, 5 aa, Synthetic construct
HYWIE

SEQ ID NO: 90 - protein, 17 aa, Synthetic construct
EILPVTGSTKYTEKFKD

SEQ ID NO: 91 - protein, 14 aa, Synthetic construct
DPHYSGSTYDAMDY

SEQ ID NO: 92 - protein, 219 aa, Synthetic construct
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - DNA, 657 bp, Synthetic construct
  source 1..657: mol_type = other DNA
aacatcgtgctgacccagagccccgcatctctggccgtgagcctgggccaaagagccaccatcagctgtagcgccagcgagagcgtggacagctatggcaacaacttcatgcactggtatcagcaaaagcctggccaaccccccaagctgctgatctacctggcaagcaacctggagagcggcgtgcccgtgaggttcagcggcagcgggagcaggaccgacttcaccctgaccatcgaccccgtggaggccgacgacgccgccacttactactgccaacagaacaacgaggacccctggaccttcggtgggggtacgaagttggagatcaagaggcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 94 - protein, 15 aa, Synthetic construct
RASESVDSYGNSFMH

SEQ ID NO: 95 - protein, 7 aa, Synthetic construct
LASNLES

SEQ ID NO: 96 - protein, 9 aa, Synthetic construct
QQNNEDPWT

SEQ ID NO: 97 - protein, 548 aa, Synthetic construct
QVQLQQSGAELMKPGASVKISCKATGYTFSHYWIEWVKQRPGHGLEWIGEILPVTGSTKYTEKFKDKATFTADTFSNTAYMQLTSLTSEDSAVYYCARDPHYSGSTYDAMDYWGQGTSVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 98 - DNA, 1644 bp, Synthetic construct
  source 1..1644: mol_type = other DNA
caggtgcagcttcaacaaagcggagcagagctgatgaagccaggcgctagtgtgaagatctcatgcaaggccaccggctacacgttcagccactattggatcgagtgggtgaaacagaggcccggtcatggcctggagtggatcggcgagatcctgcccgtgaccggctccaccaagtacaccgagaagttcaaggacaaggctaccttcaccgccgacaccttcagcaataccgcctacatgcaactgacaagcctgaccagcgaggacagcgccgtgtactactgtgccagggaccctcactacagcggcagcacctacgacgccatggactactggggccagggcaccagcgtaacagtgagcgccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 99 - protein, 220 aa, Synthetic construct
EVQLQQSGAELAKPGTSVKMSCKASGYTFISYWMNWVKQRPGQGLEWIGAINPSDGYTEYNQKFKDKAIMTADRSSSTAYMQLSSLTSEDSALYYCARYGGYFDYWGQGTTLTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 100 - DNA, 660 bp, Synthetic construct
  source 1..660: mol_type = other DNA
gaagtgcagctccaacaatccggtgccgaacttgccaaacccggaacctcagtgaagatgtcctgtaaggcttctgggtatactttcatttcctattggatgaactgggtgaagcaacgcccaggacaaggcttggaatggatcggtgcaataaatccctccgacggttataccgaatacaaccagaaattcaaggacaaagcaatcatgacggctgaccgctccagtagcaccgcgtatatgcagctttccagtcttacgagtgaagactctgcgctgtattactgcgctaggtatggaggctacttcgattattggggccagggcacaacgttgacagtcagcagcgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 101 - protein, 5 aa, Synthetic construct
SYWMN

SEQ ID NO: 102 - protein, 17 aa, Synthetic construct
AINPSDGYTEYNQKFKD

SEQ ID NO: 103 - protein, 7 aa, Synthetic construct
YGGYFDY

SEQ ID NO: 104 - protein, 219 aa, Synthetic construct
DIVLTQSPASLAVSLGQRATISCRASESVEYFGTSLMQWYQQKPGQPPKLLIYAASNVESGVPARFSGSGSGTDFSLNIHPVEEDDIAMYFCQQSREVPYTFGGGSKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 657 bp, Synthetic construct
  source 1..657: mol_type = other DNA
gacatcgtgttgactcaatcacccgctagccttgccgtaagtttgggtcagcgggccacgatcagttgtcgcgccagtgagtcagtcgaatacttcgggacatcattgatgcaatggtatcaacagaagccgggacagccgcccaaattgcttatctatgccgcgtccaatgtcgaaagtggagtaccggcacgcttctctggaagcggtagcggtactgactttagcttgaatatccatcccgtggaagaagatgacattgctatgtacttctgccagcagtccagagaggtcccttatacgttcggtggtggaagcaaattggagattaagcgccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 106 - protein, 15 aa, Synthetic construct
RASESVEYFGTSLMQ

SEQ ID NO: 107 - protein, 7 aa, Synthetic construct
AASNVES

SEQ ID NO: 108 - protein, 9 aa, Synthetic construct
QQSREVPYT

SEQ ID NO: 109 - protein, 542 aa, Synthetic construct
EVQLQQSGAELAKPGTSVKMSCKASGYTFISYWMNWVKQRPGQGLEWIGAINPSDGYTEYNQKFKDKAIMTADRSSSTAYMQLSSLTSEDSALYYCARYGGYFDYWGQGTTLTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 110 - DNA, 1626 bp, Synthetic construct
  source 1..1626: mol_type = other DNA
gaagtgcagctccaacaatccggtgccgaacttgccaaacccggaacctcagtgaagatgtcctgtaaggcttctgggtatactttcatttcctattggatgaactgggtgaagcaacgcccaggacaaggcttggaatggatcggtgcaataaatccctccgacggttataccgaatacaaccagaaattcaaggacaaagcaatcatgacggctgaccgctccagtagcaccgcgtatatgcagctttccagtcttacgagtgaagactctgcgctgtattactgcgctaggtatggaggctacttcgattattggggccagggcacaacgttgacagtcagcagcgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 111 - protein, 223 aa, Synthetic construct
QVQLQQSGPELVRPGVSVKISCKGSGYTFIDYAIHWVKESHAKSLEWIGVISAYSGDTNYNQKFKGKATMTVDKSSNTAYLELARLTSEDSAIYYCARGGWLLLSFDYWGQGTTLTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 112 - DNA, 669 bp, Synthetic construct
  source 1..669: mol_type = other DNA
caggtccaactgcagcagtcagggcctgagctggtgaggcctggggtctcagtgaagatttcctgcaagggttccggctacacattcattgattatgctatacactgggtgaaggagagtcatgcaaagagtctagagtggattggagttattagtgcttactctggtgacacaaactacaaccagaagtttaagggcaaggccacaatgactgttgacaaatcctccaacacagcctatttggaacttgccagattgacatctgaggattctgccatctattactgtgcaagagggggatggttactactctcctttgactactggggccaaggcaccactctcacagtctcctcggccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 113 - protein, 5 aa, Synthetic construct
DYAIH

SEQ ID NO: 114 - protein, 17 aa, Synthetic construct
VISAYSGDTNYNQKFKG

SEQ ID NO: 115 - protein, 10 aa, Synthetic construct
GGWLLLSFDY

SEQ ID NO: 116 - protein, 220 aa, Synthetic construct
DIVMTQSPLSLPVSLGDQASISCRSSQSLVHSNGNNYLHWYLQKSGQAPKLLIYKVSNRFSGVPDRFSGSGSGTDFTLKISRVEAEDLGVYFCSQSTHVPLTFGGGTKVELRARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 660 bp, Synthetic construct
  source 1..660: mol_type = other DNA
gatatcgtgatgacccaaagtccactctccctgcctgtcagtcttggagatcaagcctccatctcttgcagatcaagtcagagccttgtacacagcaatggaaataactatttacattggtacctgcagaagtcaggccaggctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctctcacgttcggtggaggcacaaaggtcgaattgagggcacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 118 - protein, 16 aa, Synthetic construct
RSSQSLVHSNGNNYLH

SEQ ID NO: 119 - protein, 7 aa, Synthetic construct
KVSNRFS

SEQ ID NO: 120 - protein, 9 aa, Synthetic construct
SQSTHVPLT

SEQ ID NO: 121 - protein, 545 aa, Synthetic construct
QVQLQQSGPELVRPGVSVKISCKGSGYTFIDYAIHWVKESHAKSLEWIGVISAYSGDTNYNQKFKGKATMTVDKSSNTAYLELARLTSEDSAIYYCARGGWLLLSFDYWGQGTTLTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGGSDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNHHHHHH

SEQ ID NO: 122 - DNA, 1635 bp, Synthetic construct
  source 1..1635: mol_type = other DNA
caggtccaactgcagcagtcagggcctgagctggtgaggcctggggtctcagtgaagatttcctgcaagggttccggctacacattcattgattatgctatacactgggtgaaggagagtcatgcaaagagtctagagtggattggagttattagtgcttactctggtgacacaaactacaaccagaagtttaagggcaaggccacaatgactgttgacaaatcctccaacacagcctatttggaacttgccagattgacatctgaggattctgccatctattactgtgcaagagggggatggttactactctcctttgactactggggccaaggcaccactctcacagtctcctcggccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcggccgctggaggttcagacagcttttccgccaaccaagaaataaggtattccgaggtgacgccttatcatgttacgagtgtgtggaccaagggcgttacacccccggcgaactttacgcagggagaggacgtgttccacgcgccctatgtcgctaatcaaggttggtacgacataactaagacgtttaatggtaaagatgacttgctttgtggcgcagccacggcaggaaacatgctgcattggtggttcgatcaaaacaaggatcaaattaagcgctaccttgaggagcatcctgagaaacaaaagatcaacttcaatggggaacagatgttcgatgttaaagaggcgatagataccaaaaatcatcaactcgactccaaactcttcgagtattttaaggagaaggcgttcccgtatctgtcaacaaagcacttgggggtcttcccggatcatgttattgatatgtttatcaatggatataggctctccctgacaaaccatggccccaccccagtaaaggaaggctccaaagatccacggggcggcatttttgatgccgtttttacgcgaggggaccaatcaaaactcctgaccagccggcatgacttcaaagaaaaaaacttgaaagagataagtgacttgataaagaaagagctcaccgaagggaaggctttgggactctctcacacttacgcaaacgtgcgcatcaatcatgtaatcaatctgtggggggccgatttcgatagcaatggaaatcttaaggcgatctatgtaacggattccgattctaatgcgagcattggtatgaaaaagtatttcgtgggagttaactcagctggtaaggtcgctatttccgcaaaagagatcaaggaggacaacattggtgcgcaagttttgggtctctttacgctcagcacaggccaagattcctggaatcagacgaatcatcatcaccatcaccat

SEQ ID NO: 123 - protein, 15 aa, Synthetic construct
GGGGSGGGGSGGGGS

SEQ ID NO: 124 - protein, 4 aa, Synthetic construct
LPET

(19) United States
(12) Patent Application Publication
(10) Pub. No.: US 2026/0258388 A1
(43) Pub. Date: Sep. 3, 2026

(54) COMPOSITIONS AND METHODS FOR TARGETED IDES TREATMENT OF IGG-RELATED DISORDERS

(71) Applicant: The Regents of the University of Michigan, Ann Arbor, MI (US)

(72) Inventors: Benjamin TOURDOT, Ann Arbor, MI (US); Peter TESSIER, Ann Arbor, MI (US); Colin GREINEDER, Ann Arbor, MI (US)

(21) Appl. No.: 18/877,808

(22) PCT Filed: Jun. 22, 2023
(86) PCT No.: PCT/US2023/068902

§ 371 Date: Dec. 20, 2024

Related U.S. Application Data
(60) Provisional application No. 63/354,623, filed on Jun. 22, 2022.
(60) Provisional application No. 63/354,989, filed on Jun. 23, 2022.

(51) Int. Cl. C12N 9/52; A61K 38/00

(52) U.S. Cl. CPC C12N 9/52; C12Y 304/2201; A61K 38/00; C07K 2319/01; C07K 2319/30

(57) ABSTRACT

Provided herein are fusions comprising targeted immunoglobulin G-degrading enzyme of Streptococcus pyogenes (IdeS) and methods of treating/preventing pathogenic IgG-related disorders therewith.

### CROSS-REFERENCE TO RELATED APPLICATIONS

This application claims the benefit of U.S. Provisional Patent Application No. 63/354,623, filed on Jun. 22, 2022, and U.S. Provisional Patent Application No. 63/354,989, filed on Jun. 23, 2022, both of which are incorporated by reference herein.

### SEQUENCE LISTING STATEMENT

The contents of the electronic sequence listing titled UM_40255_601.xml (Size: 146,539 bytes; and Date of Creation: Jun. 22, 2023) is herein incorporated by reference in its entirety.

### FIELD

Provided herein are fusions comprising targeted immunoglobulin G-degrading enzyme of Streptococcus pyogenes (IdeS) and methods of treating/preventing pathogenic IgG-related disorders therewith.

### BACKGROUND

Antibody-mediated autoimmune disorders, estimated to occur in approximately 2.5% of the population, are a heterogeneous group of diseases predominantly characterized by self-reacting immunoglobulin G (IgG) damaging otherwise healthy cells (Refs. A1-A2; incorporated by reference in their entireties).

In the case of immune thrombocytopenia (ITP), there is a well-defined pathogenic antibody-mediated platelet clearance, which can predispose patients to severe, life-threatening bleeding. Most ITP therapies aim to either directly or indirectly reduce pathogenic antibody levels. This, in turn, leads to an increase in platelet count and therefore, reduced disease severity. This is primarily accomplished via immunosuppressive therapies, including corticosteroids and B-cell depletion. Unfortunately, these treatments are associated with well-known iatrogenic complications. For example, immunosuppressive therapies can suppress normal immune function in patients, conferring a heightened risk for infection (refs. 3-4; incorporated by reference in their entireties). Moreover, chronic use of high-dose corticosteroids can lead to side effects including hyperglycemia, myopathy, osteoporosis, glaucoma, and psychiatric disturbances. Therefore, due to these complications, alternative treatment approaches to treat ITP are needed.

Autoimmune hemolytic anemia (AIHA) occurs when antibodies (i.e., IgG) directed against the person's own red blood cells (RBCs) cause them to burst (lyse), leading to an insufficient number of oxygen-carrying red blood cells in the circulation. The lifetime of the RBCs is reduced from the normal 100-120 days to just a few days in serious cases. The intracellular components of the RBCs are released into the circulating blood and into tissues, leading to some of the characteristic symptoms of this condition.

An acute hemolytic transfusion reaction (AHTR), also called immediate hemolytic transfusion reaction, is a life-threatening reaction to receiving a blood transfusion. AHTRs occur within 24 hours of the transfusion and can be triggered by a few milliliters of blood. The reaction is triggered by host antibodies (e.g., IgG) destroying donor red blood cells. AHTR typically occurs when there is an ABO blood group incompatibility, and is most severe when type A donor blood is given to a type O recipient. Early acute hemolytic transfusion reactions are typically characterized by fever, which may be accompanied by rigors (chills). Mild cases are also typically characterized by abdominal, back, flank, or chest pain. More severe cases may be characterized by shortness of breath, low blood pressure, hemoglobinuria, and may progress to shock and disseminated intravascular coagulation. In anesthetized or unconscious patients, hematuria (blood in the urine) may be the first sign of AHTR. Other symptoms include nausea, vomiting, and wheezing.

IdeS is a cysteine protease secreted by S. pyogenes that facilitates evasion of the humoral immune response by cleaving the heavy chain of IgG, generating a F(ab′)₂ and two Fc fragments (Ref. A5; incorporated by reference in its entirety). Since IdeS can selectively and rapidly neutralize the Fc-mediated effector function of IgG from all 4 subclasses of human IgG, it has been developed into a pharmacotherapeutic agent for treating IgG-mediated disorders. In passive murine models of autoimmune diseases, IdeS ameliorates a wide range of IgG-driven disorders including ITP and heparin-induced thrombocytopenia (HIT) (Refs. A6-A7; incorporated by reference in their entireties).

In humans, IdeS has been studied as a pharmacologic therapy in several clinical trials. Although effective, IdeS has certain limitations in its use. First, IdeS dose-dependently removes greater than 95% of IgG from circulation in Phase I dose-escalation studies in healthy subjects. While the IdeS-mediated removal of IgG was transient, recovery of normal IgG levels needed for humoral immune response may take up to 4 weeks (Ref. A8; incorporated by reference in its entirety). Second, the global nonspecific removal of IgG places the patient at risk for severe infection due to hypogammaglobulinemia necessitating use of prophylactic antibiotics in IdeS clinical trials (Ref. A8; incorporated by reference in its entirety). Finally, for some disease states, such as patients with anti-glomerular basement membrane (GBM) antibodies, the pathogenic IgG returned to toxic levels 7 days after IdeS treatment and over 50% of patients resumed plasmapheresis or immunoadsorption treatments (Ref. A9; incorporated by reference in its entirety). Yet, additional doses of IdeS are not recommended for patients with GBM-antibodies due to concern about the development of anti-IdeS antibodies that could neutralize IdeS enzymatic activity or trigger an immune hypersensitivity reaction (Ref. A9; incorporated by reference in its entirety). Taken together, the current literature demonstrates that although IdeS treatment in humans can result in a significant reduction in pathogenic IgG, novel strategies are needed to reduce its immunogenicity and global hypogammaglobulinemia.

In clinical trials, IdeS plasma concentrations greater than ~100 nM caused almost complete removal of circulating IgG (>95%), while plasma concentrations less than 25 nM did not induce significant IgG cleavage. Similarly, the immunogenicity of IdeS was also dose-dependent with only higher (>100 nM) but not lower (<25 nM) plasma concentrations resulting in detectable anti-IdeS antibodies (Ref. A8; incorporated by reference in its entirety). During life-threatening autoimmune complications such as severe bleeding or thrombosis, the immediate removal of all IgG may be warranted; however, the increased immunogenicity and potential for infection may be unacceptable risks in individuals with milder yet still significantly symptomatic autoimmune disease. Thus, strategies that can reduce the concentration of IdeS used to treat IgG-mediated immune diseases may cause less non-specific antibody cleavage and lower the likelihood of provoking an immune response potentially providing an avenue for expanding clinical indications for the use of this approach.

### SUMMARY

Provided herein are fusions comprising targeted immunoglobulin G-degrading enzyme of Streptococcus pyogenes (IdeS) and methods of treating/preventing pathogenic IgG-related disorders therewith.

In some embodiments, provided herein are compositions comprising an immunoglobulin-G degrading enzyme fused to a targeting moiety capable of specifically binding to a cell surface marker. In some embodiments, the immunoglobulin-G degrading enzyme is an immunoglobulin-G degrading enzyme of S. pyogenes (IdeS) polypeptide. In some embodiments, the IdeS polypeptide has at least 70% (e.g., 70%, 75%, 80%, 85%, 90%, 95%, 100%, or ranges or values therebetween) sequence identity to SEQ ID NO: 1 and is capable of cleaving human IgG. In some embodiments, the targeting moiety is an antibody fragment. In some embodiments, the antibody fragment is an scFv or Fab.

In some embodiments, targeting moiety is capable of binding to an erythrocyte surface marker.

In some embodiments, the targeting moiety is capable of binding human glycophorin A. In some embodiments, the targeting moiety is an antibody fragment derived from a YTH 89.1 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 4-6. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 2. In some embodiments, the targeting moiety comprises a polypeptide having CDRs having at least 70% sequence identity with SEQ ID NOS: 9-11. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 7. In some embodiments, the targeting moiety comprises a heavy chain variable region with at least 70% sequence identity with SEQ ID NO: 2 and a light chain variable region with at least 70% (e.g., 70%, 75%, 80%, 85%, 90%, 95%, 100%, or ranges or values therebetween) sequence identity with SEQ ID NO: 7. In some embodiments, the targeting moiety comprises a first variable region comprising a CDR1 of SEQ ID NO: 4, a CDR2 of SEQ ID NO: 5 and CDR3 of SEQ ID NO: 6 and a second variable region comprising a CDR1 of SEQ ID NO: 9, a CDR2 of SEQ ID NO: 10 and CDR3 of SEQ ID NO: 11.

In some embodiments, the targeting moiety is capable of binding human Wr^{b} antigen. In some embodiments, the targeting moiety is an antibody fragment derived from a Wr^{b} monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 16-18. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 14. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 21-23. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 19. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 14 and a light chain variable region with at least 70% (e.g., 70%, 75%, 80%, 85%, 90%, 95%, 100%, or ranges or values therebetween) sequence identity with SEQ ID NO: 19. In some embodiments, the targeting moiety comprises a first variable region comprising a CDR1 of SEQ ID NO: 16, a CDR2 of SEQ ID NO: 17 and CDR3 of SEQ ID NO: 18 and a second variable region comprising a CDR1 of SEQ ID NO: 21, a CDR2 of SEQ ID NO: 22 and CDR3 of SEQ ID NO: 23.

In some embodiments, the targeting moiety is capable of binding human Rh17 antigen. In some embodiments, the targeting moiety is an antibody fragment derived from a Rh17 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 28-30. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 26. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 33-35. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 31. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 26 and a light chain variable region with at least 70% (e.g., 70%, 75%, 80%, 85%, 90%, 95%, 100%, or ranges or values therebetween) sequence identity with SEQ ID NO: 31. In some embodiments, the targeting moiety comprises a first variable region comprising a CDR1 of SEQ ID NO: 28, a CDR2 of SEQ ID NO: 29 and CDR3 of SEQ ID NO: 30 and a second variable region comprising a CDR1 of SEQ ID NO: 33, a CDR2 of SEQ ID NO: 34 and CDR3 of SEQ ID NO: 35.

In some embodiments, the targeting moiety is capable of binding to a platelet surface marker. In some embodiments, the targeting moiety is capable of binding human FcγRIIA. In some embodiments, the targeting moiety is an antibody fragment derived from an IV.3 monoclonal antibody. In some embodiments, the targeting moiety comprises a polypeptide having comprises a first variable region comprising a CDR1 of SEQ ID NO: 65, a CDR2 of SEQ ID NO: 66 and CDR3 of SEQ ID NO: 67 and a second variable region comprising a CDR1 of SEQ ID NO: 68, a CDR2 of SEQ ID NO: 69 and CDR3 of SEQ ID NO: 70. In some embodiments, the targeting moiety comprises a polypeptide comprises at least 70% (e.g., 70%, 75%, 80%, 85%, 90%, 95%, 100%, or ranges or values therebetween) sequence identity with one of SEQ ID NOS: 62-64.

In some embodiments, the targeting moiety is capable of binding to an endothelial cell surface marker.

In some embodiments, the targeting moiety is capable of binding human PECAM. In some embodiments, the targeting moiety is an antibody fragment derived from a Ab37 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 77-79. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 75. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 82-84. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 80. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 75 and a light chain variable region with at least 70% (e.g., 70%, 75%, 80%, 85%, 90%, 95%, 100%, or ranges or values therebetween) sequence identity with SEQ ID NO: 80. In some embodiments, the targeting moiety comprises a polypeptide having comprises a first variable region comprising a CDR1 of SEQ ID NO: 77, a CDR2 of SEQ ID NO: 78 and CDR3 of SEQ ID NO: 79 and a second variable region comprising a CDR1 of SEQ ID NO: 82, a CDR2 of SEQ ID NO: 83 and CDR3 of SEQ ID NO: 84.

In some embodiments, the targeting moiety is capable of binding human PECAM. In some embodiments, the targeting moiety is an antibody fragment derived from a Ab62 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 89-91. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 87. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 94-96. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 92. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 87 and a light chain variable region with at least 70% (e.g., 70%, 75%, 80%, 85%, 90%, 95%, 100%, or ranges or values therebetween) sequence identity with SEQ ID NO: 92. In some embodiments, the targeting moiety comprises a polypeptide having comprises a first variable region comprising a CDR1 of SEQ ID NO: 89, a CDR2 of SEQ ID NO: 90 and CDR3 of SEQ ID NO: 91 and a second variable region comprising a CDR1 of SEQ ID NO: 94, a CDR2 of SEQ ID NO: 95 and CDR3 of SEQ ID NO: 96.

In some embodiments, the targeting moiety is capable of binding human ICAM-1. In some embodiments, the targeting moiety is an antibody fragment derived from a R6.5 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 113-115. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 111. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 118-120. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 116. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 111 and a light chain variable region with at least 70% (e.g., 70%, 75%, 80%, 85%, 90%, 95%, 100%, or ranges or values therebetween) sequence identity with SEQ ID NO: 116. In some embodiments, the targeting moiety comprises a polypeptide having comprises a first variable region comprising a CDR1 of SEQ ID NO: 113, a CDR2 of SEQ ID NO: 114 and CDR3 of SEQ ID NO: 115 and a second variable region comprising a CDR1 of SEQ ID NO: 118, a CDR2 of SEQ ID NO: 119 and CDR3 of SEQ ID NO: 120.

In some embodiments, the targeting moiety is capable of binding to a cartilage cell surface marker. In some embodiments, the targeting moiety is capable of binding human collagen type II. In some embodiments, the targeting moiety is an antibody fragment derived from a M2.139 monoclonal antibody. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 101-103. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 99. In some embodiments, the targeting moiety comprises a polypeptide having CDRs having at least 70% (e.g., 70%, 75%, 80%, 85%, 90%, 95%, 100%, or ranges or values therebetween) sequence identity with SEQ ID NOS: 106-108. In some embodiments, the targeting moiety comprises a polypeptide comprises at least 70% (e.g., 70%, 75%, 80%, 85%, 90%, 95%, 100%, or ranges or values therebetween) sequence identity with SEQ ID NO: 104. In some embodiments, the targeting moiety comprises a heavy chain variable region with at least 70% (e.g., 70%, 75%, 80%, 85%, 90%, 95%, 100%, or ranges or values therebetween) sequence identity with SEQ ID NO: 99 and a light chain variable region with at least 70% (e.g., 70%, 75%, 80%, 85%, 90%, 95%, 100%, or ranges or values therebetween) sequence identity with SEQ ID NO: 104. In some embodiments, the targeting moiety comprises a polypeptide having comprises a first variable region comprising a CDR1 of SEQ ID NO: 101, a CDR2 of SEQ ID NO: 102 and CDR3 of SEQ ID NO: 103 and a second variable region comprising a CDR1 of SEQ ID NO: 106, a CDR2 of SEQ ID NO: 107 and CDR3 of SEQ ID NO: 108.

In some embodiments, provided herein are methods of treating degrading pathogenic IgG on circulating blood cells comprising administering an IdeS/targeting moiety composition described herein to a subject. In some embodiments, administering the composition prevents or reduces destruction of blood cells by cell-bound IgG.

In some embodiments, provided herein are methods of treating a disease or condition mediated by pathogenic IgG binding to red blood cells comprising administering an IdeS/targeting moiety composition to a subject in need thereof. In some embodiments, the subject suffers from or is at risk of autoimmune hemolytic anemia (wAIHA), IgG-mediated hemolytic transfusion reaction (HTR), or hemolytic disease of the fetus and newborn (HDFN).

In some embodiments, provided herein are methods of treating a disease or condition mediated by pathogenic IgG binding to platelets comprising administering an IdeS/targeting moiety composition described herein to a subject in need thereof. In some embodiments, the subject suffers from or is at risk of immune thrombocytopenia (ITP).

In some embodiments, provided herein are method of treating a disease or condition mediated by pathogenic IgG binding to endothelial comprising administering an IdeS/targeting moiety composition described herein to a subject in need thereof. In some embodiments, the subject suffers from or is at risk of immune vasculitis, Goodpasture's (anti-GMB) disease, or organ transplant rejection.

In some embodiments, provided herein are methods of preventing organ transplant rejection comprising administering an IdeS/targeting moiety composition described herein to an organ to be transplanted, a subject to receive an organ transplant, or a subject following organ transplant.

In some embodiments, provided herein are methods of treating a disease or condition mediated by pathogenic IgG binding to cartilage comprising administering an IdeS/targeting moiety composition described herein to a subject in need thereof. In some embodiments, the subject suffers from or is at risk of autoimmune arthritis. In some embodiments, the autoimmune arthritis is rheumatoid arthritis.

In some embodiments, the administration is followed by a subsequent administration of untargeted IdeS to the subject.

In some embodiments, the compositions herein are administered by any suitable route of administration. In some embodiments, the composition is administered intravenously.

In some embodiments, provided herein is the use of an effective dose of a fusion described herein for treating or preventing an IgG mediated disease or condition.

In some embodiments, provided herein is the use of a fusion described herein in the manufacture of a medicament for use in a method of treating or preventing an IgG mediated disease or condition.

### BRIEF DESCRIPTION OF THE DRAWINGS

FIG. 1A-F. Characterization of the binding properties of scIV.3. A) Representative histograms of FAM-conjugated scIV.3 (scIV.3-FAM; 10 nM) incubated with washed platelets from WT mice which do not express human FcγRIIA (hFcγRII^{null}) or human FcγRIIA transgenic mice (hFcγRIIA^{TGN}). Increasing concentrations scIV.3-FAM were incubated with washed platelets from hFcγRIIAₙᵤₗₗ or hFcγRIIA_{TGN} mice. n=3 B) Representative histograms of increasing concentrations of scIV.3-FAM incubated with THP-1, a human monocytic like cell. THP-1 cells or human platelets were incubated with increasing concentrations of scIV.3-FAM data reported as percentage of max MFI (40 nM) for each cell. n=3-4C) Representative microscopic images (63× objective) of human platelets (CD41⁺) spread on fibrinogen, treated with control or commercial full-length IV.3 (cIV.3; 30 nM), and then stained with scIV.3-FAM (5 nM). D) Flow cytometry was used to quantify the binding of scIV.3-FAM to human platelets following the incubation with or without cIV.3. Data n=4; mean±SD; **P<0.01 E) Human whole blood was incubated with increasing concentrations of scIV.3-FAM and normalized to cIV.3 (30 nM) F) Human platelets were treated with scIV.3 (50 nM) or control for 15 minutes prior to stimulation with mouse anti-human CD9 antibodies, a FcγRIIA specific agonist. Data n=5; mean±SD; *P<0.05, ***P<0.001). Two-way ANOVA statistical analysis with Bonferroni correction was performed.

FIG. 2A-D. Generation and characterization of recombinant scIV.3-IdeS. A) Representative histograms of washed human platelets treated with 10 nM of either scIV.3-IdeS (purple) or scIV.3 (light blue). His-tagged recombinant protein and Alexa 488 conjugated mouse anti-His antibody. Increasing concentrations of either His-tagged scIV.3-IdeS or scIV.3 were incubated with washed human platelets and the binding was assessed by flow cytometry. B) Microscopic images of platelets spread on fibrinogen treated with control or commercial IV.3 (cIV.3), and co-stained with scIV.3-IdeS (5 nM) and anti-CD41. C) Surface bound His-tagged scIV.3-IdeS was quantified by flow cytometry every 30 minutes for 90 minutes. D) Representative Coomassie stained SDS-PAGE gel used to evaluate IgG cleavage following the incubation of IgG with predetermined concentrations of recombinant proteins (scIV.3, IdeS, or scIV.3-IdeS) for 1 hour at 37° C. IdeS cleaves IgG in a two-step process first generating a single-cleaved IgG (scIgG) and then a double-cleaved F(ab′)₂. The cleaved Fc fragment from both single or double cleaved IgG was quantified by a Licor Odyssey CLx Infrared Imaging System as a measure of IdeS activity. The relative fluorescence units of the Fc fragment were reported for 4 independent experiments. Data mean±SD; *P<0.05,

FIG. 3A-C. scIV.3-IdeS inhibits IgG-mediated platelet aggregation more potently than scIV.3 alone. A) Platelet-rich plasma was incubated with scIV.3 (20 nM), scIV.3-IdeS (20 nM) or control and then stimulated with mouse anti-human CD9 (Ms anti-hCD9; 1 μg/mL), ADP (20 μM) or Collagen (1 μg/mL). Representative tracings for Ms anti-hCD9 and collagen stimulated platelets were included and maximum aggregation for 3-4 independent experiments was reported B) Washed platelets treated for 15 minutes with 1, 2.5, or 20 nM of scIV.3 or scIV.3-IdeS were stimulated with ms anti-hCD9 (1 μg/mL) and maximum aggregation was reported. Statical difference between treated and control (0 nm) denoted by #P<0.05 C) Platelets treated for 15 minutes with predetermined concentrations of scIV.3 or scIV.3-IdeS were stimulated with rabbit (rb) anti-hCD9 (1 μg/mL) and maximum aggregation reported. Data shown as mean±SD (One-way ANOVA; n=3-4). ****P<0.0001.

FIG. 4A-E. scIV.3-IdeS bound to platelet FcγRIIA cleaves antiplatelet antibodies and prevents in vitro phagocytosis. A) Washed human platelets treated with 5 nM of scIV.3, IdeS, scIV.3-IdeS or vehicle control were incubated with antiplatelet antibodies (rabbit anti-human CD41 or CD42b) for 30 minutes, and then stained with a CoraLite 594 (CL594)-conjugated mouse anti-rabbit heavy chain specific antibody. The amount of full-length antiplatelet antibody bound to the surface of platelets was quantified by flow cytometry and normalized to the MFI of platelets treated with buffer control (Ctrl) for each donor. B) Representative Western blot of three independent experiments for human IgG following incubation of platelet poor plasma (PPP) with increasing concentrations of scIV.3-IdeS-coated platelets (Coated-Platelets; (18.7-150×10⁶/mL). PPP was also incubated with control (Ctrl), untreated platelets (plts) or IdeS (1 μM). IdeS treated sample lack the full-length IgG heavy chain (arrow; upper-band) but have a Fc cleavage product (arrow; lower band). Coated-platelets without PPP do not have detectable IgG at the dilution used. C) CFSE-stained human platelets treated with 5 nM of scIV.3 or scIV.3-IdeS were incubated with equal amounts of rabbit anti-human CD41 and CD42b antibodies and then were added to THP-1 cells. THP-1 cells were washed to remove excess platelets and stained with a platelet specific (PE-conjugated CD42a) antibody to distinguish THP-1 cells that had adhered (CFSE⁺/CD42a⁺) or internalized (CFSE⁺/CD42a⁻) platelets. The number of THP-1 cells with either adhered or internalized platelets were quantified by gating on CFSE⁺ THP-1 cells. THP-1 cells that were CFSE⁺ and CD42a⁻ were quantified as THP-1 cells with only internalized platelets. D) Mice expressing human Fc receptors were treated with control or IV.3-IdeS (10 μg), then IP injected with 10 or 20 μg of rabbit anti-mouse platelet sera (RAMS). Platelet counts were performed 24 hours post antiplatelet IgG injection and were normalized to pre-IgG injection (pre) measurements. E) The amount of surface bound His-tagged scIV.3-IdeS on mouse platelets was quantified by flow cytometry 0.5, 2 and 24 hours post IV.3-IdeS treatment. Data represent mean±SD; (One-way ANOVA); *P, 0.05, ***P, 0.001.

FIG. 5A-E. scIV.3-IdeS bound to platelet FcγRIIA cleaves antiplatelet antibodies from HIT and ITP sera and prevents in vitro phagocytosis. A) PF4-dependent P-selectin expression assays were performed using sera from 5 HIT patients and platelets from healthy donors treated with 5 nM of scIV.3-IdeS or vehicle control. B) Washed human platelets treated with 5 nM of scIV.3-IdeS or vehicle control were incubated with sera from ITP patients, and then stained with a FITC-conjugated mouse anti-human Fc specific antibody. The amount of full-length antiplatelet antibody bound to the surface of platelets was quantified by flow cytometry. Data represents mean±SD (two-way ANOVA; n=4-6). C) The PF4-dependent P-selectin expression assay was performed with platelets treated with scIV.3 or scIV.3-IdeS using sera from a HIT patient (HIT5) D) Platelets treated with scIV.3 or scIV.3-IdeS were incubated with sera from a ITP patient (ITP4), then stained with a FITC-conjugated mouse anti-human Fc specific antibody, and the amount of full-length antiplatelet antibody bound to the surface of platelets was quantified by flow cytometry. E) CFSE-stained human platelets treated with scIV.3-IdeS (5 nM) or vehicle control were incubated with sera from ITP patients and then were added to preactivated THP-1 cells. THP-1 cells were washed to remove excess platelets and stained with a platelet specific (PE-conjugated CD42a) antibody to distinguish THP-1 cells that had adhered (CFSE⁺/CD42a⁺) or internalized (CFSE⁺/CD42a⁻) platelets. The number of THP-1 cells with either adhered or internalized platelets were quantified by gating on CFSE⁺ THP-1 cells. THP-1 cells that were CFSE⁺ and CD42a⁻ were quantified as THP-1 cells with only internalized platelets. Data represents mean=SD. (1-way ANOVA; n=4). *P<0.05.

FIG. 6A-C. Quantitative, HPLC-based IdeS activity assay. A.) Schematic showing the assay reaction, in which IdeS cleaves recombinant human IgG1 labeled site-specifically at the C-terminus (“fluoro-IgG”). Middle panels show representative SEC traces and demonstrate appearance of product (“fluoro-Fc”) over time. Fluoro-Fc signal is normalized to the total fluorescent signal to give % cleavage. B.) % cleavage over time at different concentrations—the rate is approximately linear over the first 30-60 minutes. C.) Calculation of IdeS specific activity, expressed as % cleavage/min/fmol enzyme.

FIG. 7A-E. Synthesis and characterization of Ter119 scFv-IdeS. A.) SDS-PAGE and B.) SEC HPLC of 1. IdeS, 2. Ter119 scFv, 3. scFv-IdeS fusion. C.) Binding assay using mouse RBC ghosts shows saturable binding of Ter119 scFv (Kd=30 nM) and scFv-IdeS (Kd=26 nM). D). SDS-PAGE showing cleavage of human IgG by scFv-IdeS and IdeS at different concentrations. The appearance of the characteristic band of the cleaved heavy chain is shown. E.) HPLC-based IdeS activity assay shows roughly equivalent specific activities of IdeS and scFv-IdeS fusion protein.

FIG. 8. Quantitative comparison of IdeS specific activity in antibodies of different species and isotype.

FIG. 9A-B. Ter119 scFv-IdeS cleaves RBC-bound IgGs and blocks agglutination. Agglutination assays performed using ‘humanized’ Ter119 mAb (A) and 34-3C mAb (B). All mAbs were used at 0.5 nM with 10 nM anti-human F(ab′)₂ as a secondary.

FIG. 10A-C. Blood PK and biodistribution of Ter119 scFv-IdeS. A.) Erythrocyte anchoring markedly extends the blood circulation time and B.) alters the distribution of IdeS within the blood, from >80% in the plasma to >98% bound to RBCs at 1 hour post-injection. C.) 1 hour organ biodistribution of scFv-IdeS and IdeS.

FIG. 11A-E. scFv-IdeS provides potent protection in a murine model of human IgG-mediated hemolysis. Mice were injected with 2 mg/kg humanized Ter119 mAb vs. control human IgG1 vs. Ter119 F(ab′)₂. For treatment, mice received IdeS or scFv-IdeS 30 min before Ter119 mAb, with Ter1 19 scFv as a control. A.) Hemoglobin, mean±SEM, *-p<0.05, ***-p<0.001. B.) Histograms of RBC volume showing marked reticulocytosis C.) peripheral blood smears, showing polychromasia and anisocytosis (red arrows) and D.) liver and E.) spleen histopathology, showing extramedullary hematopoesis, all significantly improved in mice treated with scFv-IdeS.

FIG. 12. Human Fab-IdeS agglutination assays.

FIG. 13. Pharmacokinetics of anti-RBC (Ter119) IdeS constructs (Fab vs. scFv vs. untargeted).

FIG. 14. In vitro selectivity assay demonstrating the cleavage of RBC-bound and soluble IgG by RBC targeted and untargeted IdeS.

FIG. 15. Design of assay to (1) determine the ability of anti-endothelial cell (EC) Fab-IdeS to cleave EC-bound IgG and (2) determine the selectivity of anti-EC Fab IdeS for EC-bound vs soluble IgG.

FIG. 16A-E. In vivo selectivity assay A-C.) Principle of the FLAG-IgG ELISA, which uses an Fc-specific anti-human IgG antibody for capture and an HRP-conjugated anti-FLAG antibody to quantitate intact vs. IdeS-cleaved FLAG-IgG in mouse plasma. D.) Time course of in vivo selectivity experiment. ~40-fold difference in dose of IdeS vs. Fab-Ides reflects difference in potency of the two proteins. E.) % cleavage of soluble vs. RBC-bound IgG (normalized to untreated) at 2 hr, 6 hr, and a range of timepoints shows the selectivity of Fab-Ides, but not untargeted IdeS, for RBC-bound IgG, mean±SEM with n=6 per group.

FIG. 17A-B. Immunogenicity experiments A) Schematic of sandwich ELISA using IdeS or Fab-IdeS coated plates. Titer was calculated as the highest dilution of plasma to give 2× background. B) Results of immunogenicity experiment, in which mice were given repeated doses of IdeS or Ter119 Fab-IdeS (black arrows). Red arrow indicates timing of the last dose of IdeS, as additional doses were found to cause anaphylaxis, mean±SEM with n=4 per group.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the terms “subject at risk for a disease,” or “subject at risk for a condition,” refers to a subject with one or more risk factors for developing the disease/condition. Depending upon the specific disease, risk factors may include, but are not limited to, gender, age, genetic predisposition, environmental exposures, infections, and previous incidents of diseases, lifestyle, etc.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., orally, intravenously, subcutaneously, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) (e.g., a fusion construct herein and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “instructions for administering,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like, refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

As used herein, the term “antibody” refers to a whole antibody molecule or a fragment thereof (e.g., fragments such as Fab, Fab′, and F(ab′)₂), unless specified otherwise; an antibody may be polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256:495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352:624-628; and Marks et al. (1991) J. Mol. Biol. 222:581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

### DETAILED DESCRIPTION

Provided herein are fusions comprising targeted immunoglobulin G-degrading enzyme of Streptococcus pyogenes (IdeS) and methods of treating/preventing pathogenic IgG-related disorders therewith.

In some embodiments, provided herein are IdeS polypeptides fused to a targeting moiety. In some embodiments, the targeting moiety is capable of binding to target molecule (e.g., a peptide, a small molecule, a lipid, a carbohydrate, a protein (e.g., cell surface marker, cell surface receptor, etc.). In some embodiments, the targeting moiety is an antibody, antibody fragment (e.g., Fab, Fab′, F(ab′)₂, Fv, scFv, Fd, diabodies, etc.), DARPin, anticalin, nanobody, aptamer, affimer, analyte binding domain of protein, etc. In some embodiments, the targeting moiety is a single polypeptide chain (e.g., an scFv). In some embodiments, the targeting moiety comprises multiple peptide and/or polypeptide chains (e.g., a Fab). In embodiments in which a targeting moiety is a multi-polypeptide complex (e.g., the light and heavy chains of a Fab), the IdeS polypeptide may be bound to one of the polypeptides, and one or more of the other polypeptides associated with the IdeS fusion to form a targeted IdeS construct. In some embodiments, the targeting polypeptide fused to the IdeS polypeptide forms a complex with one or more additional targeting polypeptides to form a targeting complex capable of binding to the target. In some embodiments, the targeting moiety is not cleavable by IdeS. In some embodiments, the target molecule is present on a particular class of cells or tissues (e.g., red blood cells, platelets, cartilage, endothelial cells, etc.).

Targeting IdeS to the surface of cells relevant to the specific IgG-mediated disorder is a strategy to decrease the amount of pathogenic IgG without a concomitant generalized collateral global IgG degradation or induction of an immune reaction to IdeS.

In experiments conducted during development of embodiments herein, FcγRIIA, a low-affinity IgG receptor, was chosen as an exemplary target (e.g., a platelet-based binding target) for IdeS based on the following properties: 1) selective expression on a distinct cell type (e.g., platelets, monocytes and neutrophils (ref. A10; incorporated by reference in its entirety)); 2) proximity to autoantibody targets (e.g., two of the most common autoantibody platelet targets in ITP patients: αIIbβ3 and GPIb/V/IX (ref. A11; incorporated by reference in its entirety)); 3) minimal importance in normal hemostasis (ref. A12; incorporated by reference in its entirety); and 4) involvement in pathological autoantibody-mediated platelet activation (such as HIT) (ref. A13: incorporated by reference in its entirety). Moreover, a well-characterized, high affinity, function-blocking humanized monoclonal antibody specific for human FcγRIIA designated IV.3 was available (refs. A14-A16: incorporated by reference in their entireties). Using a site-specific bioconjugation strategy, the C-terminus of a single-chain variable fragment (scFv) of IV.3 was covalently conjugated to the N-terminus of IdeS, producing a single protein product, scIV.3-IdeS (e.g., SEQ ID NO: 66), capable of anchoring IdeS to the surface of FcγRIIA-expressing cells. Remarkably, platelets decorated with scIV.3-IdeS cleaved platelet bound-IgG, resulting in a decrease in platelet phagocytosis in vitro, without inducing proteolytic cleavage of non-pathogenic IgG. Furthermore, scIV.3-IdeS was capable of mitigating thrombocytopenia in a passive mouse model of ITP.

While non-targeted IdeS therapies are promising strategies for treating autoimmune diseases, their side-effect profile limits current clinical use to sensitized kidney transplant patients. In experiments conducted during development of embodiments herein, it was found that recombinant IdeS, modified to bind to FcγRIIA (scIV.3-IdeS), retained the ability to bind FcγRIIA with high-affinity, cleave antiplatelet IgG, block FcγRIIA-mediated platelet activation, and prevent phagocytosis in vitro without a significant decrease in nonpathogenic IgG. Moreover, in a passive murine model of ITP, scIV.3-IdeS prevented platelet clearance. These results indicate the utility of markedly expanding the clinical indications for applying this approach in human IgG-mediated disorders.

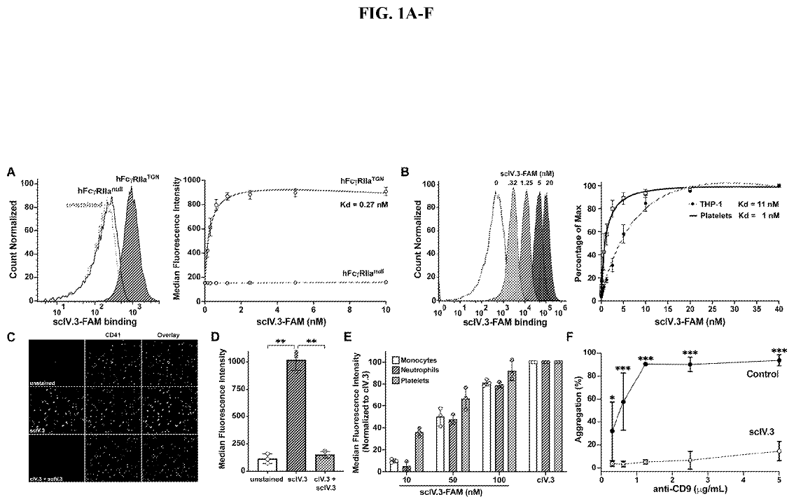

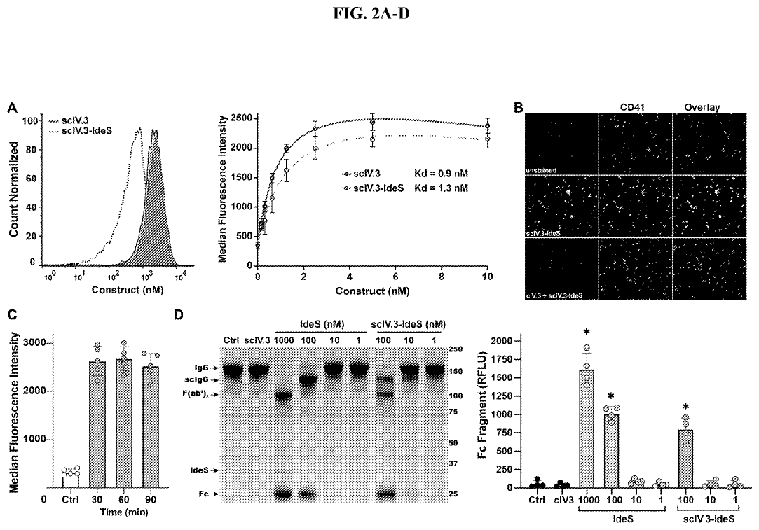

While both Fc-receptor dependent and independent platelet clearance occurs in patients with ITP, the low-affinity IgG receptors (FcγRIIA and FcγRIIIA) on macrophages in the reticuloendothelial system are primarily responsible for the clearance of IgG-coated platelets in an Fc receptor-dependent manner (refs. A27-29; incorporated by reference in their entireties). scIV.3 was demonstrated to bind platelets and monocytes with low nanomolar affinity in vitro (FIG. 1). The scIV.3-IdeS complex was retained on the platelet surface for the duration of in vitro testing (1.5 hours) and was detected on the surface of platelets in mice 2 hours after injection in vivo (FIG. 2D). Thus, scIV.3-IdeS is well-positioned to cleave the Fc fragment of IgG from the platelet's surface. In some embodiments, localization of IdeS to the surface of platelets is achieved by targeting platelet-specific receptors such as αIIb, GPVI, or GPIb/V/IX, for example, with fusions containing antibody fragments that specifically bind these targets.

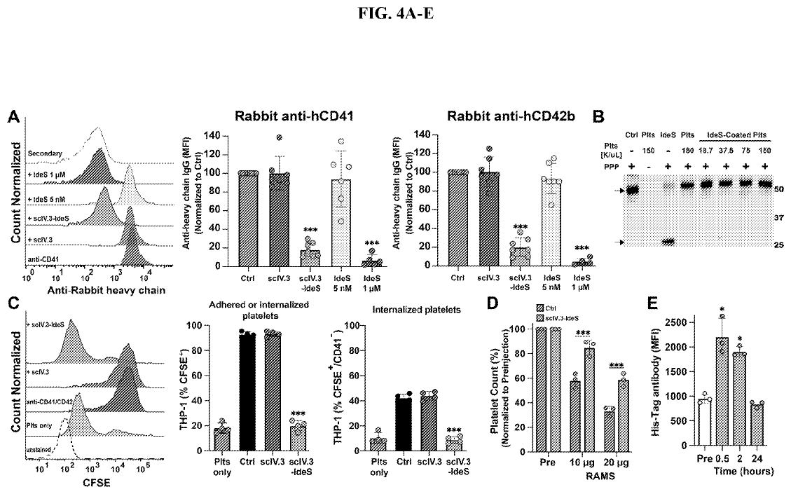

It was observed that prophylactic intravenous injection of scIV.3-IdeS into mice with human Fc receptors reduced platelet clearance in a passive model of ITP (FIG. 4D). The ability of scIV.3-IdeS to protect mice from thrombocytopenia in models with polyclonal rabbit anti-mouse platelet antibodies against multiple platelet antigens indicates that scIV.3-IdeS broadly neutralizes antiplatelet IgG regardless of their specificity.

Platelet clearance and activation can occur in autoimmune disorders through the formation of immune complexes that activate platelets via FcγRIIA (refs. A32-33; incorporated by reference in their entireties). Previously studies have demonstrated that full-length or Fab fragments of IV.3 can block IgG-mediated platelet activation and thrombosis (ref. A34; incorporated by reference in its entirety). Experiments conducted during development of embodiments herein demonstrate that scIV.3 and scIV.3-IdeS block FcγRIIA-mediated platelet activation via anti-CD9 antibodies and HIT patient sera. ScIV.3-IdeS was more effective at blocking FcγRIIA-mediated platelet activation than scIV.3 at concentrations in which platelet FcγRIIA was not fully occupied. The cleavage of pathogenic IgG complexes by platelets coated in scIV.3-IdeS can neutralize IgG complexes from activating platelets, extending to platelet protection even after scIV.3-IdeS has been cleared.

In some embodiments, including scIV.3-IdeS as part of a regimen first-line pharmacotherapeutic alone or in conjunction with corticosteroids provides a minimally invasive therapeutic approach to help raise platelet counts in patients with ITP without negative impact on host defense. In some embodiments, scIV.3-IdeS finds use in the treatment of acute IgG-driven platelet diseases with clearly defined Fc-dependent pathogenesis such as fetal and neonatal alloimmune thrombocytopenia, vaccine-induced thrombocytopenia and thrombosis, HIT, and pediatric ITP.

The experiments conducted during development of embodiments herein using scIV.3-IdeS to target FcγRIIA on platelets provides a demonstration of the utility of targeted IdeS for the treatment of IgG-based autoantibody conditions.

Selective removal of pathogenic IgG (e.g., by scIV.3-IdeS, by Wr^{b}-IdeS, Rh17-IdeS, Ter11-IdeS, etc.) alone or in conjunction with other treatment for diseases or conditions to be treated, provides are treatment for patients suffering from IgG-mediated immune disorders. (e.g., autoimmune platelet, endothelial cell, cartilage, or RBC disorders). In some embodiments, cell specific targeting of IdeS to affected tissue improves treatments while minimizing side effects.

In some embodiments, provided herein are fusion constructs comprising an immunoglobulin-G degrading enzyme fused to a targeting moiety.

In preferred embodiments, the immunoglobulin-G (IgG) degrading enzyme fused to a targeting moiety is Immunoglobulin G-degrading enzyme of Streptococcus pyogenes (IdeS) or a variant thereof (e.g., at least 70% sequence identity to SEQ ID NO: 1) that is capable of proteolytic cleavage of human IgG. In some embodiments, the IgG degrading enzyme is specific for IgG (e.g., does not cleave other immunoglobulins or other human proteins). In some embodiments, the IgG degrading enzyme is capable of hydrolyzing IgG at a position in the hinge region, after glycine 2326, of the heavy chain of IgG (both heavy chains of the antibody). IdeS is a well-characterized enzyme (see, e.g., Wenig et al. PNAS (2004). 101 (50) 17371-17376; incorporated by reference in its entirety). In some embodiments, the IgG degrading enzyme exhibits the IgG degrading activity of IdeS but contains C- or N-terminal truncations of the IdeS amino acid sequence. some embodiments, the IgG degrading enzyme exhibits the IgG degrading activity of IdeS and has at least 70% (e.g., 70%, 75%, 80%, 85%, 90%, 95%, 98%, 99%, 100%, or ranges therebetween) sequence identity to all or a portion (e.g., at least 100, 150, 200, 250, or 300 amino acids in length) of SEQ ID NO: 1.

In preferred embodiments, the targeting moiety is a targeting polypeptide or a complex of targeting polypeptides. In some embodiments, the targeting moiety is an antibody or antibody fragment that is incapable of being cleaved or otherwise degraded by the immunoglobulin-G degrading enzyme of the fusion. In particular embodiments, the targeting moiety is an antibody or antibody fragment that lacks the sequence or structural element that is cleaved by the IgG-degrading enzyme of the fusion. In some embodiments, the targeting polypeptide lacks a hinge region or contains substitutions in the hinge region to prevent cleavage of the targeting moiety by the IgG-degrading enzyme. In some embodiments, the targeting moiety lacks a hinge region. In some embodiments, the targeting polypeptide lacks an Fc region. In some embodiments, the targeting moiety lacks a hinge region and an Fc region. In some embodiments, the targeting moiety is a single chain variable fragment (scFv). In some embodiments, the targeting moiety is an antigen binding fragment (Fab). In embodiments in which the targeting moiety is a targeting complex (e.g., comprises two or more peptides/polypeptides), the IgG-degrading enzyme (e.g., IdeS) is fused to one or both of the components of the targeting complex

In some embodiments, the targeting moiety is capable of binding to a cell surface marker (e.g., protein, peptide, lipid, small molecule, etc.) that is displayed on the surface of a cell. Exemplary cell types for targeting in embodiments herein include circulating blood cells (e.g., platelets, RBCs, leukocytes, etc.), cartilage and other joint components (e.g., collagen and components of the other extracellular matrix, chondrocytes, synovium and synoviocytes, etc.), endothelial cells, basement membrane components (e.g., type IV collagen), and cells within transplanted organs. In some embodiments, the targeting moiety binds to a cell surface marker that is displayed on a particular cell type or class of cells.

In some embodiments, the targeting moiety is capable of binding to a cell surface marker (e.g., protein) that is displayed on the surface of a circulating blood cell.

In some embodiments, the targeting moiety is capable of binding to a cell surface marker (e.g., protein) that is displayed on the surface of a platelet. In some embodiments, the platelet cell surface marker is unique to platelets (e.g., not expressed by and/or displayed on the surface of other cell types (in some embodiments, the markers may be present on platelet precursor cells)). In some embodiments, the marker is abundant on the surface of platelets. In some embodiments, the targeting moiety binds to platelet-specific marker, like FcγRIIa, glycoprotein IIb (GPIIb, or CD41, glycoprotein Ib alpha (GPIbα, or CD42b), glycoprotein Ib beta (GPIbβ, or CD42c) glycoprotein V (GPV, or CD42d) or glycoprotein IX (GPIX, or CD42a). In some embodiments, the targeting moiety is an antibody or antibody fragment derived from the monoclonal antibody IV.3. In some embodiments, the targeting moiety lacks an IdeS cleavage site. In some embodiments, the IV-3-based targeting moiety (targeting polypeptide or complex) is an scFv or Fab. In some embodiments, the targeting moiety is an IV.3-based antibody or antibody fragment (e.g., scFv) with a first variable region (e.g., heavy chain variable region) comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of the monoclonal antibody IV.3 (e.g., SEQ ID NO: 65, 66, and/or 67). In some embodiments, the targeting moiety is an IV.3-based antibody or antibody fragment with a heavy chain variable region comprising complementarity determining regions comprising sequences of SEQ ID NO: 65, 66, or and/or 67. In some embodiments, the targeting moiety is an IV.3-based antibody or antibody fragment (e.g., scFv) with a second variable region (e.g., light chain variable region) comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of the monoclonal antibody IV.3 (e.g., SEQ ID NO: 68, 69, and/or 70). In some embodiments, the targeting moiety is an IV.3-based antibody or antibody fragment (e.g., scFv) with a second variable region (e.g., light chain variable region) comprising complementarity determining regions comprising sequences of SEQ ID NO: 68, 69, or and/or 70. In some embodiments, the targeting moiety comprises sequences having at least 70% (e.g., 70%, 75%, 80%, 85%, 90%, 95%, 95%, 99%, 100% or ranges therebetween) sequence identity to one of SEQ ID NOS: 62, 63, or 64. In some embodiments, the targeting moiety is an IV.3-based antibody or antibody fragment (e.g., scFv, Fab, etc.). In some embodiments, exemplary FcγRIIa-targeted IdeS constructs have at least 70% (e.g., 70%, 75%, 80%, 85%, 90%, 95%, 95%, 99%, 100% or ranges therebetween) sequence identity to SEQ ID NO: 73 or 74.

In some embodiments, the targeting moiety is capable of binding to a cell surface marker (e.g., protein) that is displayed on the surface of a red blood cell. In some embodiments, the RBC surface marker is unique to erythrocytes (e.g., not expressed by and/or displayed on the surface of other cell types (in some embodiments, the markers may be present on RBC precursor cells)). In some embodiments, the marker is abundant on the surface of RBCs. In some embodiments, the targeting moiety binds to erythrocyte-specific marker human glycophorin A, the human Wright b (Wr^{b}) epitope, or human Rh17/Hr₀ epitope on RhCE.

In some embodiments, the RBC-specific targeting moiety is an antibody or antibody fragment derived from the YTH 89.1 monoclonal antibody. In some embodiments, the targeting moiety lacks an IdeS cleavage site. In some embodiments, provided herein is an IdeS polypeptide fused (e.g., directly or via a linker sequence) to a targeting polypeptide (e.g., a scFv) or a component of a targeting complex (e.g., a Fab).

In some embodiments, the targeting moiety binds specifically to human glyphorin A. In some embodiments, the targeting moiety is an antibody, antibody fragment, or other specific binding agent that recognizes and binds human glyphorin A. In some embodiments, the targeting moiety is a YTH 89.1-based antibody or antibody fragment (e.g., scFv or Fab). In some embodiments, the targeting moiety is not cleaved by IdeS or any other IgG-degrading enzyme used in the system or methods. In some embodiments, the targeting moiety is an YTH 89.1-based antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of the monoclonal antibody YTH 89.1 (e.g., SEQ ID NO: 4, 5, and/or 6). In some embodiments, the targeting moiety is an YTH 89.1-based antibody or antibody fragment with a heavy chain variable region comprising complementarity determining regions comprising sequences of SEQ ID NO: 4, 5, or and/or 6. In some embodiments, the targeting moiety is a YTH 89.1-based Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 2. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a YTH V_{H}-C_{H1} sequence (SEQ ID NO: 2). In some embodiments, the targeting moiety comprises a YTH V_{H}-C_{H1} sequence (SEQ ID NO: 2). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 3. In some embodiments, the targeting moiety is an YTH 89.1-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of the monoclonal antibody YTH 89.1 (e.g., SEQ ID NO: 9, 10, and/or 11). In some embodiments, the targeting moiety is an YTH 89.1-based antibody or antibody fragment with a light chain variable region comprising complementarity determining regions comprising sequences of SEQ ID NO: 9, 10, or and/or 11. In some embodiments, the targeting moiety is a YTH 89.1-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 7. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a YTH V_{L}-C_{L} sequence (SEQ ID NO: 7). In some embodiments, the targeting moiety comprises a YTH V_{L}-C_{L} sequence (SEQ ID NO: 7). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 8.

In some embodiments, the targeting moiety is a Fab comprising a heavy chain comprising the CDRs of SEQ ID NOS: 4-6 and a light chain comprising the CDRs of SEQ ID NOS: 9-11. In some embodiments, the heavy chain comprises at least 70% (e.g., 70%, 75%, 80%, 85%, 90%, 95%, 95%, 99%, 100% or ranges therebetween) sequence identity to SEQ ID NO: 2 and the light chain comprises at least 70% (e.g., 70%, 75%, 80%, 85%, 90%, 95%, 95%, 99%, 100% or ranges therebetween) sequence identity to SEQ ID NO: 7. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 12 or is encoded by a nucleic acid having at least 70% (e.g., 70%, 75%, 80%, 85%, 90%, 95%, 95%, 99%, 100% or ranges therebetween) sequence identity SEQ ID NO: 13.

In some embodiments, the RBC-specific targeting moiety is an antibody or antibody fragment that binds the Wright b (Wr^{b}) antigen. In some embodiments, the targeting moiety lacks an IdeS cleavage site. In some embodiments, provided herein is an IdeS polypeptide fused (e.g., directly or via a linker sequence) to a targeting polypeptide (e.g., a scFv) or a component of a targeting complex (e.g., a Fab).

In some embodiments, the targeting moiety binds specifically to a Wr^{b} antigen. In some embodiments, the targeting moiety is an antibody, antibody fragment, or other specific binding agent that recognizes and binds Wr^{b}. In some embodiments, the targeting moiety is an antibody or antibody fragment (e.g., scFv or Fab) comprising sequences and binding activity of a Wr^{b} monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of a Wr^{b} monoclonal antibody (e.g., SEQ ID NO: 16, 17, and/or 18). In some embodiments, the targeting moiety is an antibody or antibody fragment with a heavy chain variable region comprising complementarity determining regions comprising sequences of SEQ ID NO: 16, 17, and/or 18. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 14. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Wr^{b} V_{H}-C_{H1} sequence (SEQ ID NO: 14). In some embodiments, the targeting moiety comprises a Wr^{b} V_{H}-C_{H1} sequence (SEQ ID NO: 14). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 15. In some embodiments, the targeting moiety is a Wr^{b}-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a Wr^{b} monoclonal antibody (e.g., SEQ ID NO: 21, 22, and/or 23). In some embodiments, the targeting moiety is an Wr^{b}-based antibody or antibody fragment with a light chain variable region comprising complementarity determining regions comprising sequences of SEQ ID NO: 21, 22, and/or 23. In some embodiments, the targeting moiety is a Wr^{b}-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 19. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Wr^{b} V_{L}-C_{L} sequence (SEQ ID NO: 19). In some embodiments, the targeting moiety comprises a Wr^{b} V_{L}-C_{L} sequence (SEQ ID NO: 19). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 20.

In some embodiments, the targeting moiety is a Fab comprising a heavy chain comprising the CDRs of SEQ ID NOS: 16-18 and a light chain comprising the CDRs of SEQ ID NOS: 21-23. In some embodiments, the heavy chain comprises at least 70% (e.g., 70%, 75%, 80%, 85%, 90%, 95%, 95%, 99%, 100% or ranges therebetween) sequence identity to SEQ ID NO: 14 and the light chain comprises at least 70% (e.g., 70%, 75%, 80%, 85%, 90%, 95%, 95%, 99%, 100% or ranges therebetween) sequence identity to SEQ ID NO: 19. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 24 or is encoded by a nucleic acid having at least 70% (e.g., 70%, 75%, 80%, 85%, 90%, 95%, 95%, 99%, 100% or ranges therebetween) sequence identity SEQ ID NO: 25.

In some embodiments, the RBC-specific targeting moiety is an antibody or antibody fragment that binds the Rh17 antigen. In some embodiments, the targeting moiety lacks an IdeS cleavage site. In some embodiments, provided herein is an IdeS polypeptide fused (e.g., directly or via a linker sequence) to a targeting polypeptide (e.g., a scFv) or a component of a targeting complex (e.g., a Fab).

In some embodiments, the targeting moiety binds specifically to a Rh17 antigen. In some embodiments, the targeting moiety is an antibody, antibody fragment, or other specific binding agent that recognizes and binds Rh17. In some embodiments, the targeting moiety is an antibody or antibody fragment (e.g., scFv or Fab) comprising sequences and binding activity of a Rh17 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of a Rh17 monoclonal antibody (e.g., SEQ ID NO: 28, 29, and/or 30). In some embodiments, the targeting moiety is an antibody or antibody fragment with a heavy chain variable region comprising complementarity determining regions comprising sequences of SEQ ID NO: 28, 29, and/or 30. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 26. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Rh17 V_{H}-C_{H1} sequence (SEQ ID NO: 26). In some embodiments, the targeting moiety comprises a Rh17 V_{H}-C_{H1} sequence (SEQ ID NO: 26). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 27. In some embodiments, the targeting moiety is a Rh17-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a Rh17 monoclonal antibody (e.g., SEQ ID NO: 33, 34, and/or 35). In some embodiments, the targeting moiety is an Rh17-based antibody or antibody fragment with a light chain variable region comprising complementarity determining regions comprising sequences of SEQ ID NO: 33, 34, and/or 35. In some embodiments, the targeting moiety is a Rh17-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 31. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Rh17V_{L}-C_{L} sequence (SEQ ID NO: 31). In some embodiments, the targeting moiety comprises a Rh17 V_{L}-C_{L} sequence (SEQ ID NO: 31). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 32.

In some embodiments, the targeting moiety is a Fab comprising a heavy chain comprising the CDRs of SEQ ID NOS: 28-30 and a light chain comprising the CDRs of SEQ ID NOS: 33-35. In some embodiments, the heavy chain comprises at least 70% (e.g., 70%, 75%, 80%, 85%, 90%, 95%, 95%, 99%, 100% or ranges therebetween) sequence identity to SEQ ID NO: 26 and the light chain comprises at least 70% (e.g., 70%, 75%, 80%, 85%, 90%, 95%, 95%, 99%, 100% or ranges therebetween) sequence identity to SEQ ID NO: 31. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 36 or is encoded by a nucleic acid having at least 70% (e.g., 70%, 75%, 80%, 85%, 90%, 95%, 95%, 99%, 100% or ranges therebetween) sequence identity SEQ ID NO: 37.

In some embodiments, the RBC-specific targeting moiety is an antibody or antibody fragment that binds the mouse Ter119 antigen. In some embodiments, the targeting moiety lacks an IdeS cleavage site. In some embodiments, provided herein is an IdeS polypeptide fused (e.g., directly or via a linker sequence) to a targeting polypeptide (e.g., a scFv) or a component of a targeting complex (e.g., a Fab).

In some embodiments, the targeting moiety binds specifically to a mouse Ter119 antigen. In some embodiments, the targeting moiety is an antibody, antibody fragment, or other specific binding agent that recognizes and binds mouse Ter119 antigen. In some embodiments, the targeting moiety is an antibody or antibody fragment (e.g., scFv or Fab) comprising sequences and binding activity of a Ter119 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of a Ter119 monoclonal antibody (e.g., SEQ ID NO: 40, 41, and/or 42). In some embodiments, the targeting moiety is an antibody or antibody fragment with a heavy chain variable region comprising complementarity determining regions comprising sequences of SEQ ID NO: 40, 41, and/or 42. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 38. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ter119 V_{H}-C_{H1} sequence (SEQ ID NO: 38). In some embodiments, the targeting moiety comprises a Ter119 V_{H}-C_{H1} sequence (SEQ ID NO: 38). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 39. In some embodiments, the targeting moiety is a Ter119-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a Ter119 monoclonal antibody (e.g., SEQ ID NO: 45, 46, and/or 47). In some embodiments, the targeting moiety is an Ter119-based antibody or antibody fragment with a light chain variable region comprising complementarity determining regions comprising sequences of SEQ ID NO: 45, 46, and/or 47. In some embodiments, the targeting moiety is a Ter119-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 43. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ter119 V_{L}-C_{L} sequence (SEQ ID NO: 43). In some embodiments, the targeting moiety comprises a Ter119 V_{L}-C_{L} sequence (SEQ ID NO: 43). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 44.

In some embodiments, the targeting moiety is a Fab comprising a heavy chain comprising the CDRs of SEQ ID NOS: 40-42 and a light chain comprising the CDRs of SEQ ID NOS: 45-47. In some embodiments, the heavy chain comprises at least 70% (e.g., 70%, 75%, 80%, 85%, 90%, 95%, 95%, 99%, 100% or ranges therebetween) sequence identity to SEQ ID NO: 38 and the light chain comprises at least 70% (e.g., 70%, 75%, 80%, 85%, 90%, 95%, 95%, 99%, 100% or ranges therebetween) sequence identity to SEQ ID NO: 43. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS is fused to a heavy chain of the targeting Fab (e.g., a polypeptide having at least 70% (e.g., 70%, 75%, 80%, 85%, 90%, 95%, 95%, 99%, 100% or ranges therebetween) sequence identity SEQ ID NO: 48, a polypeptide encoded by a nucleic acid having at least 70% (e.g., 70%, 75%, 80%, 85%, 90%, 95%, 95%, 99%, 100% or ranges therebetween) sequence identity SEQ ID NO: 49, etc.).

In some embodiments, the RBC-specific targeting moiety is a 34-3C-based antibody or antibody fragment that binds the same antigen as the mouse monoclonal antibody 34-3C. In some embodiments, the targeting moiety lacks an IdeS cleavage site. In some embodiments, provided herein is an IdeS polypeptide fused (e.g., directly or via a linker sequence) to a targeting polypeptide (e.g., a scFv) or a component of a targeting complex (e.g., a Fab).

In some embodiments, the targeting moiety binds specifically to the mouse antigen of monoclonal antibody 34-3C. In some embodiments, the targeting moiety is an antibody, antibody fragment, or other specific binding agent that recognizes and binds the antigen of monoclonal antibody 34-3C. In some embodiments, the targeting moiety is an antibody or antibody fragment (e.g., scFv or Fab) comprising sequences and binding activity of monoclonal antibody 34-3C. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of a monoclonal antibody 34-3C (e.g., SEQ ID NO: 52, 53, and/or 54). In some embodiments, the targeting moiety is an antibody or antibody fragment with a heavy chain variable region comprising complementarity determining regions comprising sequences of SEQ ID NO: 52, 53, and/or 54. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 50. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a 34-3C V_{H}-C_{H1} sequence (SEQ ID NO: 50). In some embodiments, the targeting moiety comprises a 34-3C V_{H}-C_{H1} sequence (SEQ ID NO: 50). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 51. In some embodiments, the targeting moiety is a 34-3C-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a 34-3C monoclonal antibody (e.g., SEQ ID NO: 57, 58, and/or 59). In some embodiments, the targeting moiety is an 34-3C-based antibody or antibody fragment with a light chain variable region comprising complementarity determining regions comprising sequences of SEQ ID NO: 57, 58, and/or 59. In some embodiments, the targeting moiety is a 34-3C-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 55. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a 34-3C V_{L}-C_{L} sequence (SEQ ID NO: 55). In some embodiments, the targeting moiety comprises a 34-3C V_{L}-C_{L} sequence (SEQ ID NO: 55). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 56.

In some embodiments, the targeting moiety is a Fab comprising a heavy chain comprising the CDRs of SEQ ID NOS: 52-54 and a light chain comprising the CDRs of SEQ ID NOS: 57-59. In some embodiments, the heavy chain comprises at least 70% (e.g., 70%, 75%, 80%, 85%, 90%, 95%, 95%, 99%, 100% or ranges therebetween) sequence identity to SEQ ID NO: 50 and the light chain comprises at least 70% (e.g., 70%, 75%, 80%, 85%, 90%, 95%, 95%, 99%, 100% or ranges therebetween) sequence identity to SEQ ID NO: 55. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS is fused to a heavy chain of the targeting Fab (e.g., a polypeptide having at least 70% (e.g., 70%, 75%, 80%, 85%, 90%, 95%, 95%, 99%, 100% or ranges therebetween) sequence identity SEQ ID NO: 60, a polypeptide encoded by a nucleic acid having at least 70% (e.g., 70%, 75%, 80%, 85%, 90%, 95%, 95%, 99%, 100% or ranges therebetween) sequence identity SEQ ID NO: 61, etc.).

In some embodiments, the targeting moiety is capable of binding to a cell surface marker (e.g., protein) that is displayed on the surface of an endothelial cell. In some embodiments, the endothelial cell surface marker is unique to endothelial cells (e.g., not expressed by and/or displayed on the surface of other cell types (in some embodiments, the markers may be present on endothelial precursor cells)). In some embodiments, the marker is abundant on the surface of endothelial cells. In some embodiments, the targeting moiety binds to endothelial-cell-specific marker human platelet endothelial cell adhesion molecule (PECAM) or intercellular adhesion molecule 1 (ICAM-1).

In some embodiments, the targeting moiety binds specifically to a PECAM. In some embodiments, the targeting moiety is an antibody, antibody fragment, or other specific binding agent that recognizes and binds PECAM. In some embodiments, the targeting moiety is an antibody or antibody fragment (e.g., scFv or Fab) comprising sequences and binding activity of an anti-PECAM monoclonal antibody. In some embodiments, the targeting moiety is an antibody or antibody fragment (e.g., scFv or Fab) comprising sequences and binding activity of Ab37 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of Ab37 (e.g., SEQ ID NO: 77, 78, and/or 79). In some embodiments, the targeting moiety is an antibody or antibody fragment with a heavy chain variable region comprising complementarity determining regions comprising sequences of SEQ ID NO: 77, 78, and/or 79. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 75. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ab37 V_{H}-C_{H1} sequence (SEQ ID NO: 75). In some embodiments, the targeting moiety comprises a Ab37 V_{H}-C_{H1} sequence (SEQ ID NO: 75). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 76. In some embodiments, the targeting moiety is a Ab37-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a Ab37 monoclonal antibody (e.g., SEQ ID NO: 82, 83, and/or 84). In some embodiments, the targeting moiety is an Ab37-based antibody or antibody fragment with a light chain variable region comprising complementarity determining regions comprising sequences of SEQ ID NO: 82, 83, and/or 84. In some embodiments, the targeting moiety is a Ab37-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 80. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ab37 V_{L}-C_{L} sequence (SEQ ID NO: 80). In some embodiments, the targeting moiety comprises a Ab37 V_{L}-C_{L} sequence (SEQ ID NO: 80). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 81. In some embodiments, the targeting moiety is a Fab comprising a heavy chain comprising the CDRs of SEQ ID NOS: 77-79 and a light chain comprising the CDRs of SEQ ID NOS: 82-84. In some embodiments, the heavy chain comprises at least 70% (e.g., 70%, 75%, 80%, 85%, 90%, 95%, 95%, 99%, 100% or ranges therebetween) sequence identity to SEQ ID NO: 75 and the light chain comprises at least 70% (e.g., 70%, 75%, 80%, 85%, 90%, 95%, 95%, 99%, 100% or ranges therebetween) sequence identity to SEQ ID NO: 80. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 85 or is encoded by a nucleic acid having at least 70% (e.g., 70%, 75%, 80%, 85%, 90%, 95%, 95%, 99%, 100% or ranges therebetween) sequence identity SEQ ID NO: 86.

In some embodiments, the targeting moiety binds specifically to a PECAM. In some embodiments, the targeting moiety is an antibody, antibody fragment, or other specific binding agent that recognizes and binds PECAM. In some embodiments, the targeting moiety is an antibody or antibody fragment (e.g., scFv or Fab) comprising sequences and binding activity of an anti-PECAM monoclonal antibody. In some embodiments, the targeting moiety is an antibody or antibody fragment (e.g., scFv or Fab) comprising sequences and binding activity of Ab62 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of Ab62 (e.g., SEQ ID NO: 89, 90, and/or 91). In some embodiments, the targeting moiety is an antibody or antibody fragment with a heavy chain variable region comprising complementarity determining regions comprising sequences of SEQ ID NO: 89, 90, and/or 91. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 87. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ab62 V_{H}-C_{H1} sequence (SEQ ID NO: 87). In some embodiments, the targeting moiety comprises a Ab62 V_{H}-C_{H1} sequence (SEQ ID NO: 87). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 88. In some embodiments, the targeting moiety is a Ab62-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a Ab62 monoclonal antibody (e.g., SEQ ID NO: 94, 95, and/or 96). In some embodiments, the targeting moiety is an Ab62-based antibody or antibody fragment with a light chain variable region comprising complementarity determining regions comprising sequences of SEQ ID NO: 94, 95, and/or 96. In some embodiments, the targeting moiety is a Ab62-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 92. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a Ab62 V_{L}-C_{L} sequence (SEQ ID NO: 92). In some embodiments, the targeting moiety comprises a Ab62 V_{L}-C_{L} sequence (SEQ ID NO: 92). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 93.

In some embodiments, the targeting moiety is a Fab comprising a heavy chain comprising the CDRs of SEQ ID NOS: 89-91 and a light chain comprising the CDRs of SEQ ID NOS: 94-96. In some embodiments, the heavy chain comprises at least 70% (e.g., 70%, 75%, 80%, 85%, 90%, 95%, 95%, 99%, 100% or ranges therebetween) sequence identity to SEQ ID NO: 87 and the light chain comprises at least 70% (e.g., 70%, 75%, 80%, 85%, 90%, 95%, 95%, 99%, 100% or ranges therebetween) sequence identity to SEQ ID NO: 92. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 97 or is encoded by a nucleic acid having at least 70% (e.g., 70%, 75%, 80%, 85%, 90%, 95%, 95%, 99%, 100% or ranges therebetween) sequence identity SEQ ID NO: 98.

In some embodiments, the targeting moiety binds specifically to a ICAM-1. In some embodiments, the targeting moiety is an antibody, antibody fragment, or other specific binding agent that recognizes and binds ICAM-1. In some embodiments, the targeting moiety is an antibody or antibody fragment (e.g., scFv or Fab) comprising sequences and binding activity of an anti-ICAM-1 monoclonal antibody. In some embodiments, the targeting moiety is an antibody or antibody fragment (e.g., scFv or Fab) comprising sequences and binding activity of R6.5 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of R6.5 (e.g., SEQ ID NO: 113, 114, and/or 115). In some embodiments, the targeting moiety is an antibody or antibody fragment with a heavy chain variable region comprising complementarity determining regions comprising sequences of SEQ ID NO: 113, 114, and/or 115. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 111. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a R6.5 V_{H}-C_{H1} sequence (SEQ ID NO: 111). In some embodiments, the targeting moiety comprises a R6.5 V_{H}-C_{H1} sequence (SEQ ID NO: 111). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 112. In some embodiments, the targeting moiety is a R6.5-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a R6.5 monoclonal antibody (e.g., SEQ ID NO: 118, 119, and/or 120). In some embodiments, the targeting moiety is an R6.5-based antibody or antibody fragment with a light chain variable region comprising complementarity determining regions comprising sequences of SEQ ID NO: 118, 119, and/or 120. In some embodiments, the targeting moiety is a R6.5-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 116. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a R6.5 V_{L}-C_{L} sequence (SEQ ID NO: 116). In some embodiments, the targeting moiety comprises a R6.5 V_{L}-C_{L} sequence (SEQ ID NO: 116). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 117.

In some embodiments, the targeting moiety is a Fab comprising a heavy chain comprising the CDRs of SEQ ID NOS: 113-115 and a light chain comprising the CDRs of SEQ ID NOS: 118-120. In some embodiments, the heavy chain comprises at least 70% (e.g., 70%, 75%, 80%, 85%, 90%, 95%, 95%, 99%, 100% or ranges therebetween) sequence identity to SEQ ID NO: 111 and the light chain comprises at least 70% (e.g., 70%, 75%, 80%, 85%, 90%, 95%, 95%, 99%, 100% or ranges therebetween) sequence identity to SEQ ID NO: 116. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 121 or is encoded by a nucleic acid having at least 70% (e.g., 70%, 75%, 80%, 85%, 90%, 95%, 95%, 99%, 100% or ranges therebetween) sequence identity SEQ ID NO: 122.

In some embodiments, the targeting moiety is capable of binding to a cell surface marker (e.g., protein) that is displayed on the surface of a cartilage cell (e.g., chondrocyte). In some embodiments, the cartilage cell surface marker is unique to cartilage cells (e.g., not expressed by and/or displayed on the surface of other cell types (in some embodiments, the markers may be present on cartilage precursor cells)). In some embodiments, the marker is abundant on the surface of cartilage cells.

In some embodiments, the targeting moiety binds specifically to collagen type II. In some embodiments, the targeting moiety is an antibody, antibody fragment, or other specific binding agent that recognizes and binds collagen type II. In some embodiments, the targeting moiety is an antibody or antibody fragment (e.g., scFv or Fab) comprising sequences and binding activity of an anti-collagen type II monoclonal antibody. In some embodiments, the targeting moiety is an antibody or antibody fragment (e.g., scFv or Fab) comprising sequences and binding activity of M2.139 monoclonal antibody. In some embodiments, the targeting moiety is not cleaved by IdeS or any other IgG-degrading enzyme used in the system or methods. In some embodiments, the targeting moiety is an antibody or antibody fragment with a heavy chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the heavy chain of M2.139 (e.g., SEQ ID NO: 101, 102, and/or 103). In some embodiments, the targeting moiety is an antibody or antibody fragment with a heavy chain variable region comprising complementarity determining regions comprising sequences of SEQ ID NO: 101, 102, and/or 103. In some embodiments, the targeting moiety is a Fab comprising a heavy-chain polypeptide component with at least 70% (e.g., 70%, 75%, 80%, 85%, 90%, 95%, 95%, 99%, 100% or ranges therebetween) sequence identity to SEQ ID NO: 99. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a M2.139 V_{H}-C_{H1} sequence (SEQ ID NO: 99). In some embodiments, the targeting moiety comprises a M2.139 V_{H}-C_{H1} sequence (SEQ ID NO: 99). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 100. In some embodiments, the targeting moiety is a M2.139-based antibody or antibody fragment with a light chain variable region comprising complementarity determining regions comprising at least 70% (e.g., 70%, 75%, 80%, 85%, 90%, 95%, 95%, 99%, 100% or ranges therebetween) sequence identity to one or more of the CDRs of the light chain of a M2.139 monoclonal antibody (e.g., SEQ ID NO: 106, 107, and/or 108). In some embodiments, the targeting moiety is an M2.139-based antibody or antibody fragment with a light chain variable region comprising complementarity determining regions comprising sequences of SEQ ID NO: 106, 107, and/or 108. In some embodiments, the targeting moiety is a M2.139-based Fab comprising a light-chain polypeptide component with at least 70% (e.g., 70%, 75%, 80%, 85%, 90%, 95%, 95%, 99%, 100% or ranges therebetween) sequence identity to SEQ ID NO: 104. In some embodiments, the targeting moiety comprises a polypeptide comprising a sequence having at least 70% (e.g., 70%, 75%, 80%, 85%, 90%, 95%, 95%, 99%, 100% or ranges therebetween) sequence identity to a M2.139 V_{L}-C_{L} sequence (SEQ ID NO: 104). In some embodiments, the targeting moiety comprises a M2.139 V_{L}-C_{L} sequence (SEQ ID NO: 104). In some embodiments, the targeting moiety comprises a polypeptide encoded by a sequence having at least 70% (e.g., 70%, 75%, 80%, 85%, 90%, 95%, 95%, 99%, 100% or ranges therebetween) sequence identity to SEQ ID NO: 105.

In some embodiments, the targeting moiety is a Fab comprising a heavy chain comprising the CDRs of SEQ ID NOS: 101-103 and a light chain comprising the CDRs of SEQ ID NOS: 106-108. In some embodiments, the heavy chain comprises at least 70% (e.g., 70%, 75%, 80%, 85%, 90%, 95%, 95%, 99%, 100% or ranges therebetween) sequence identity to SEQ ID NO: 99 and the light chain comprises at least 70% (e.g., 70%, 75%, 80%, 85%, 90%, 95%, 95%, 99%, 100% or ranges therebetween) sequence identity to SEQ ID NO: 104. In some embodiments, an IgG-degrading enzyme e.g., IdeS, having at least 70% (e.g., 70%, 75%, 80%, 85%, 90%, 95%, 95%, 99%, 100% or ranges therebetween) sequence identity to SEQ ID NO: 1, etc.) is fused (e.g., directly or via a suitable linker) to one or both of the heavy and light chains of the targeting Fab. In some embodiments, an IdeS fused to a heavy chain of the targeting Fab has at least 70% (e.g., 70%, 75%, 80%, 85%, 90%, 95%, 95%, 99%, 100% or ranges therebetween) sequence identity to SEQ ID NO: 109 or is encoded by a nucleic acid having at least 70% (e.g., 70%, 75%, 80%, 85%, 90%, 95%, 95%, 99%, 100% or ranges therebetween) sequence identity SEQ ID NO: 110.

In some embodiments, the targeting moiety and the immunoglobulin-G (IgG) degrading enzyme are fused directly together. In some embodiments, the targeting moiety and the immunoglobulin-G (IgG) degrading enzyme are fused by a linker sequence. The linker sequence is an amino acid sequence of suitable length (e.g., 1-50 amino acids (e.g., 1, 2, 5, 10, 15, 20, 25, 30, 35, 40, 45, 50, or ranges therebetween)), flexibility and/or rigidity, hydrophobicity/hydrophilicity, charge/non-polarity, etc. to allow the targeting moiety to stably bind to its target and the IgG degrading enzyme to efficiently cleave IgG. Any suitable linker sequences are within the scope herein.

In some embodiments, provided herein are methods for treatment or prevention of pathogenic IgG-related disorders by the administration of the fusion constructs described herein.

In some embodiments, a subject is administered a fusion comprising an RBC targeting moiety and an IgG degrading enzyme (e.g., IdeS). In some embodiments, the RBC targeting moiety binds human glycophorin A, human Wright b (Wr^{b}) epitope, or human Rh17/Hr₀ epitope on RhCE. In some embodiments, the subject suffers from or is at risk of warm autoimmune hemolytic anemia (wAIHA), IgG-mediated hemolytic transfusion reaction (HTR), or hemolytic disease of the fetus and newborn (HDFN).

In some embodiments, a fusion comprising an RBC targeting moiety and an IgG degrading enzyme (e.g., IdeS) is administered to a subject suffering from warm autoimmune hemolytic anemia (wAIHA). wAIHA is an autoimmune disorder characterized by the premature destruction of healthy red blood cells (hemolysis). In some embodiments, the fusion is co-administered with other therapeutics for the treatment of wAIHA. Currently, other treatments for wAIHA are supportive and include corticosteroids, rituximab, immunosuppressive agents, and blood transfusions. The fusions herein may be co-administered with any therapeutics for the treatment of wAIHA and/or reduction/suppression of symptoms thereof.

In some embodiments, a fusion comprising an RBC targeting moiety and an IgG degrading enzyme (e.g., IdeS) is administered to a subject suffering from or at risk of Rh-mediated hemolytic transfusion reaction (HTR). HTRs are the clinical consequence of the immune destruction of transfused red cells. HTR typically occurs when antigen-positive red blood cells are transfused into a patient who has a clinically significant alloantibody to that antigen. Severe acute HTR (AHTR) which occur within 24 hours of the offending transfusion are typically due to intravascular hemolysis caused by complement fixing IgM antibodies. However, AHTR can be caused by extravascular red cell destruction by IgG antibodies, such as, anti-D, anti-K in patients sensitized by previous transfusions or pregnancy. Delayed HTR (DHTR) occurs 5-8 days following transfusion and are due to anamnestic or secondary immune responses in previously sensitized (‘primed’) patients in whom no antibody can be detected in the pre-transfusion sample leading to extravascular hemolysis. In some embodiments, a fusion comprising an RBC targeting moiety and an IgG degrading enzyme (e.g., IdeS) is coadministered with a transfusion to prevent IgG-mediated HTR. In some embodiments, the fusion is administered with a blood transfusion when it has been determined prior to transfusion that the subject is at risk for HTR (e.g., the subject has antibodies to an antigen on the red blood cells to be transfused). In some embodiments, methods herein comprise testing a sample (e.e.gm blood or blood product (e.g., serum, plasma, etc.) from a subject for antibodies to one or more antigens on red blood cells. In some embodiments, methods herein comprise testing a sample for blood to be transfused for one or more antigens on red blood cells. In some embodiments, a fusion comprising an RBC moiety polypeptide and an IgG degrading enzyme (e.g., IdeS) is administered following a blood transfusion (e.g., when evidence appears of HTR). HTR is characterized by the destruction of healthy red blood cells (hemolysis) in transfused blood. In some embodiments, the fusion is co-administered with other therapeutics for the treatment of HTR or suppression of symptoms of HTR. Currently, other treatments for HTR are supportive and include diuretics, blood pressure support, and treatment of disseminated intravascular coagulation (with fresh frozen plasma, cryoprecipitate, and platelet transfusion). The fusions herein may be co-administered with any therapeutics for the treatment of HTR and/or reduction/suppression of symptoms thereof.

In some embodiments, a fusion comprising an RBC targeting moiety and an IgG degrading enzyme (e.g., IdeS) is administered to a subject suffering from or at risk of hemolytic disease of the fetus and newborn (HDFN). During pregnancy, RBCs from the unborn baby can cross into the mother's blood through the placenta. HDFN occurs when the immune system of the mother these RBCs as foreign. Antibodies develop against the fetuses RBCs. These antibodies attack the RBCs in the infant or newborns blood and cause them to break down too early. HDFN develops when a mother and her unborn baby have different blood type antigens. In some embodiments, a fusion herein is administered to a pregnant mother, to a gestating fetus, or to an infant human. In some embodiments, a fusion herein is administered with other therapeutics for the treatment of HDFN including blood transfusions. The fusions herein may be co-administered with any therapeutics for the treatment of HDFN and/or reduction/suppression of symptoms thereof. In some embodiments, methods herein comprise testing a sample from a mother and/or baby (or gestating fetus) to assess the risk of HDFN.

In some embodiments, a fusion comprising platelet targeting moiety and an IgG degrading enzyme (e.g., IdeS) is administered to a subject suffering from immune thrombocytopenia (ITP). ITP is a blood disorder characterized by a decrease in the number of platelets in the blood. ITP is caused by an IgG-mediated autoimmune reaction against a subject's own platelets. ITP can develop in both children and adults. Acute thrombocytopenia purpura usually affects young children, ages 2 to 6 years old. The symptoms may follow a viral illness, such as chickenpox. The onset of acute ITP is typically sudden and the symptoms usually disappear in less than 6 months, often within a few weeks. Treatment may or may not be required. Chronic ITP may occur at any age and the symptoms last 6 months or more (e.g., 1 year, 2 years, 3 years, 4 years, 5 years, 10 years, 20 years, or more). In some embodiments, a fusion herein is administered to a subject suffering from ITP (e.g., an adult subject, an adolescent subject). In some embodiments, methods herein comprise a step of diagnosing ITP. In some embodiments, diagnostic steps include a complete medical history, physical exam, complete blood count (CBC), antiplatelet antibody test, bone marrow aspiration, etc. In some embodiments, the fusion is co-administered with other therapeutics for the treatment of ITP, including, but not limited to steroids, and intravenous IgG. The fusions herein may be co-administered with any therapeutics for the treatment of ITP and/or reduction/suppression of symptoms thereof.

In some embodiments, a fusion comprising a cartilage targeting moiety and an IgG degrading enzyme (e.g., IdeS) is administered to a subject suffering from autoimmune arthritis (e.g., rheumatoid arthritis). In some embodiments, a cartilage targeting moiety binds to an antigen on a protein within the cartilage of a subject, for example, collagen type II or modified collagen (e.g., citrinullated collagen). In some embodiments, a fusion of IdeS and an antibody or antibody fragment capable of binding to collagen type II is provided. In some embodiments, the fusions are co-administered with one or more treatments for autoimmune arthritis (e.g., rheumatoid arthritis), such as methotrexate, leflunomide, hydroxychloroquine, sulfasalazine, corticosteroids, abatacept (Orencia), adalimumab (Humira), anakinra (Kineret), certolizumab (Cimzia), etanercept (Enbrel), golimumab (Simponi, Simponi Aria), infliximab (Remicade), rituximab (Rituxan), sarilumab (Kevzara), tocilizumab (Actemra), or biosimilars thereof.

In some embodiments, a fusion comprising an endothelial cell targeting moiety and an IgG degrading enzyme (e.g., IdeS) is administered to a subject suffering from autoimmune arthritis (e.g., rheumatoid arthritis). In some embodiments, an endothelial cell targeting moiety binds to an antigen on the surface of endothelial cells, for example, endothelial cell (PECAM, or CD31, or ICAM-1, or CD54) or basement membrane proteins (type IV collagen). In some embodiments, a fusion of IdeS and an antibody or antibody fragment capable of binding to PECAM-1 and ICAM-1 is provided.

In some embodiments, a fusion comprising an endothelial cell targeting moiety and an IgG degrading enzyme (e.g., IdeS) is loaded into a donor organ just prior to transplantation or administered to the recipient during or after surgery. In some embodiments, the fusions are co-administered with one or more anti-rejection medications, such as prednisone, tacrolimus, (Prograf), cyclosporine (Neoral), mycophenolate mofetil (CellCept), imuran (Azathioprine), rapamune (Rapamycin, Sirolimus), etc.

Non-specific IdeS administration to a subject can result in induction of an immune reaction to IdeS. However, in some embodiments, targeted IdeS (e.g., the fusions herein) is less is less immunogenic than regular IdeS. In some embodiments, targeted IdeS induces tolerance to untargeted IdeS. In some embodiments, a subject is initially administered one or more doses of a targeted IdeS, followed by one or more doses of untargeted IdeS. In such embodiments, the targeted IdeS induces a tolerance in the subject to the IdeS, allowing untargeted IdeS to be administered without significant immunogenic effect.

### EXPERIMENTAL

### Example 1

Experiments were conducted during development of embodiments herein to develop fusions or IdeS linked to antibody fragments that bind erythroid-specific surface targets (i.e., no expression on non-RBC cell types). Potential targets were selected based on being expressed across the patient population, and high enough copy number to enable appropriate loading with IdeS and rapid cleavage of pathologic, cell-bound antibodies. The RhD antigen, for example, was not selected, as ~15% of the population is RhD-negative, meaning that the therapeutic would not work in this subset of the population.

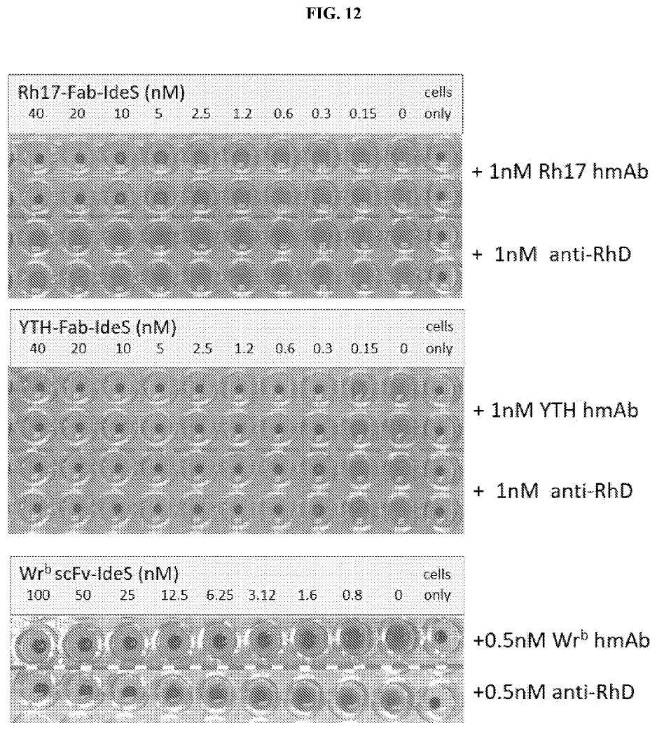

Three fusions were synthesized for initial consideration: YTH-IdeS, Wr^{b}-IdeS, and Rh17-IdeS—each of which binds to a distinct, erythroid specific target with near universal expression and relatively high copy number. YTH Fab-IdeS is derived from the YTH 89.1 monoclonal antibody (“YTH mAb”), which binds to human glycophorin A (GPA, or CD235a). GPA is erythroid specific and one of the highest copy number proteins on the surface of murine and human erythrocytes (~106 per RBC). YTH-IdeS is the direct analog of the Ter119-IdeS that has been used to target IdeS to RBC in mice. Wr^{b}-IdeS binds to the Wright b (Wr^{b}) epitope. This is a complex epitope which spans two different proteins that form a complex on the RBC membrane, band 3 and GPA. Wr^{b} is erythroid specific, nearly universal, and very high copy number (~10⁶ per RBC). Rh17-IdeS binds to the Rh17/Hr₀ epitope on RhCE. Unlike the RhD antigen, the Rh17/Hr₀ epitope is present in nearly all individuals. It is erythroid specific and has a moderate copy number (~10⁵ per RBC). Each of the three fusions (Rh17 Fab IdeS, YTH Fab-IdeS, Wr^{b} scFv-IdeS) was tested in an agglutination assay, to determine if cell-bound IdeS was able to cleave cell-bound antibody. Each fusion was tested against an antibody that binds to the same site (i.e., Rh17 mAb+Rh17-Fab IdeS) and separately using anti-RhD (RhoGAM®). Although each of the constructs was capable of cleaving cell-bound IgG, the experiments demonstrated that YTH Fab-IdeS was the most potent (FIG. 12).

### Example 2

### FcγRIIA-Targeted IgG-Degrading Enzymes Selectively Remove Pathogenic Antiplatelet Antibodies

### Materials and Methods

### Isolation of Human Platelet-Rich Plasma (PRP) and Platelets

Research involving human subjects was approved by Cincinnati Children's Hospital Medical Center Institutional Review Board. Written informed consent was obtained from all subjects before enrollment in this study. Whole blood was collected into vacutainers containing 3.2% sodium citrate (Becton, Dickinson and Company (BD) and centrifuged for 10 minutes at 200×g without brakes to obtain PRP. Where indicated, citrated PRP was supplemented with 1 mM CaCl₂) and used without adjustment of platelet count. To further isolate platelets from PRP, acid citrate dextrose (ACD; 2.5% sodium citrate tribasic, 1.5% citric acid, 2.0% D-glucose), and Prostaglandin E1 (PGE₁) 50 ng/ml (Cayman Chemicals) were added and then the samples were centrifuged for 10 minutes at 2000×g. The pelleted platelets were resuspended in Tyrode's buffer (10 mM HEPES, 12 mM NaHCO₃, 127 mM NaCl, 5 mM KCl, 0.5 mM NaH₂PO₄, 1 mM MgCl₂, and 5 mM glucose) and adjusted to 3.0×10⁸ platelets/mL, unless otherwise stated.

### Isolation of Murine Platelets

Studies involving mice were approved by the Cincinnati Children's Hospital Medical Center IACUC. Blood was drawn from the inferior vena cava of mice anesthetized with ketamine/xylazine with a 21-gauge needle into a 1 mL syringe containing 100 μL of 3.8% sodium citrate. Blood was diluted with equal volumes of Tyrode's buffer and centrifuged for 4 minutes at 200×g without brakes. The diluted PRP was transferred to a fresh tube and then equal volumes of Tyrode's buffer was added back to the blood. The samples were gently inverted and centrifuged for 4 minutes at 200×g without brakes to maximize recovery of platelets. ACD and PGE1 (50 ng/ml) were then added to the diluted PRP, and centrifuged for 5 minutes at 2000×g. The pelleted platelets were resuspended in Tyrode's buffer and adjusted to 3.0×10⁸ platelets/mL, unless otherwise stated

### Expression, Sortase Modification, and Purification

V_{H} and V_{L} sequences for IV.3 were fused with a (GGGGS)₃ (SEQ ID NO: 123) linker and purchased as a geneblock from Integrated DNA Technologies. This sequence was cloned into bacterial expression plasmid pBAD/scFv-LPETGG via NcoI/NheI restriction enzyme sites (refs. A17 and A35; incorporated by reference in their entireties). scIV.3-LPETGG protein was expressed in the periplasm of Top 1OF bacteria and purified using anti-FLAG resin (BioLegend). Purified scIV.3-LPETGG was C-terminally modified with a FAM peptide as (ref. A17; incorporated by reference in its entirety). A cDNA encoding amino acids 30-339 of S. pyogenes IdeS (NBCI WP_010922160), corresponding to the mature proteolytic enzyme (ref. A5; incorporated by reference in its entirety), was cloned into the N-terminal sortag vector, pRSET/GGG, via NdeI/EcoRI restriction enzyme sites. GGG-IdeS was expressed in BL21 bacteria and purified from cellular lysate via immobilized metal affinity chromatography (Ni-NTA agarose, Qiagen). The purity of both proteins was confirmed to be >95% by size exclusion HPLC (SEC-HPLC). The proteins were reacted with sortase-A5 at a ratio of 1:1.5 (scIV.3-LPETGG: GGG-IdeS) and the desired protein product, scIV.3-IdeS, was purified by SEC-HPLC.

### Characterization of Construct Binding by Flow Cytometry

Washed platelets (7.5×10⁶), whole blood (5 μL), or THP-1 cells (5×10⁵) were incubated with increasing concentrations of scIV.3-FAM for 30 minutes at room temperature in Tyrode's buffer supplemented with 3% fetal bovine serum. Samples were fixed with equal volumes of 4% formaldehyde, centrifuged (3 minutes at 2000×g), and resuspended in PBS. Samples were analyzed by flow cytometry, and binding was reported as median fluorescence intensity (MFI). Whole blood samples were simultaneously stained with scIV.3-FAM and cell-specific antibodies against monocytes (CD14; PE-Cy7-conjugated), neutrophils (CD66b; PerCP-CY5-conjugated), and platelets (CD42a; PE-conjugated). Where indicated, 30 nM of commercial IV.3 (StemCell Tech) was added to platelets 15 minutes prior to the addition of scIV.3 or scIV.3-IdeS.

To characterize the binding of scIV.3-IdeS, increasing concentrations of His-tagged recombinant proteins were incubated with platelets (7.5×10⁶) for 30 minutes at room temperature, followed by the addition of an Alexa Fluor 488 conjugated anti-His Tag antibody for an additional 30 minutes at room temperature. Samples were fixed with equal volumes of 4% formaldehyde for 10 minutes at ambient temperature and spun down at 1000×g for 3 minutes.

### Aggregation

A Chrono-log Model 490 4+4 aggregometer was used to measure aggregation under stirring conditions (1000 rpm) at 37° C. following the addition of the indicated agonist to a 250 μL aliquot of platelets or PRP. Platelets were stimulated with collagen (Chrono-log), ADP (Sigma-Aldrich), mouse monoclonal anti-human CD9 antibody (Beckman Coulter, IM0117, clone ALB6), or rabbit monoclonal anti-human CD9 (Abcam) antibody at concentrations indicated in the figure legend. Where appropriate, platelets or PRP were treated with scIV.3 or vehicle control for 15 minutes at 37° C., before being stimulated.

### IgG-Degradation

Human IgG (Cat #IHUIGGGF1GM, Innovative Research, Inc) was resuspended in Tyrode's buffer to a final concentration of 2 mg/mL and incubated with predetermined concentrations of recombinant proteins for an hour at 37° C. Reactions were stopped with the addition of 5× Laemmli non-reducing sample buffer (300 mM Tris-Cl pH 6.8, 10% SDS, 50% glycerol, 0.05% bromophenol blue), heated at 95° C. for 5 minutes, and separated on a 10% SDS-PAGE gel. Gels were stained with Coomassie dye (0.1% Coomassie R250, 50% Methanol, 10% Acetic Acid), destained with water, and scanned on a Li-COR Odyssey CLX imager.

### Platelet Spreading

Eight-well chambered cover glass slIdeS were coated with fibrinogen (100 μg/mL) for 1 hour at 37° C. Chambers were washed 3 times with PBS and then platelets (2×10⁶ platelets/mL) were allowed to spread for 1 hour at 37° C. Nonadherent platelets were removed via 3 PBS washes, the platelets were stained with a recombinant protein (scIV.3-FAM or scIV.3-IdeS) and Alexa Fluor 647 conjugated CD41 for 30 minutes at room temperature, washed 3 more times with PBS, and fixed with 2% formaldehyde for 10 minutes at room temperature. Where indicated, commercial IV.3 (30 nM) was added to platelets for 15 minutes prior to the addition of recombinant protein. Images of platelets were obtained on a Nikon Ti-E inverted microscope using at 63×/1.40 oil objective.

### In Vitro Antiplatelet Antibody Cleavage

For studies using commercial antiplatelet antibodies, washed platelets (7.5×10⁶) in 50 μL were incubated with the stated concentration of recombinant protein for 5 minutes at room temperature followed by the addition of 200 ng of a rabbit polyclonal antibody raised against either human CD41 (Proteintech) or human CD42b (Proteintech) for 30 minutes at 37° C. After, a CoraLite 594-conjugated mouse monoclonal antibody specific to the heavy chain of rabbit IgG (Proteintech) was incubated with platelets for 30 minutes at room temperature. Samples were then fixed and analyzed by flow cytometry. As a control, increasing concentrations of platelets treated with scIV.3-IdeS were incubated with equal volumes of autologous platelet poor plasma for 1 hour at 37° C. For studies with ITP patient sera, washed platelets (3.0×10⁶) in 50 μL were incubated with the stated concentration of recombinant protein for 5 minutes at room temperature followed by the addition 2 μL of ITP patient sera (1:25 dilution). APC-conjugated mouse monoclonal antibody specific to the Fc fragment of human IgG (Invitrogen) was incubated with platelets for 30 minutes at room temperature. Samples were then fixed and analyzed by flow cytometry.

### In Vitro Phagocytosis

THP-1 cells, a human monocyte-like cell line, were plated at 1×10⁶ cells/mL in 500 μL in a 24-well plate. THP-1 cells were differentiated for approximately 20 hours by the addition of 2 ng/ml TGF-β1 (R&D) and 50 nM 1,25-(OH) 2-vitamin D3 (Sigma) to culture media (RPMI 1640 medium, 10% fetal calf serum, 2 mM L-glutamine, 100 units/mL penicillin and 100 μg/mL streptomycin (Life Technologies). Wells were washed once with calcium-free PBS and the remaining adherent macrophages were activated by the addition of culture media containing 15 ng/mL phorbol-myristate acetate (PMA, Sigma) for 30 minutes. After PMA activation, the media was replaced with fresh culture media. Concurrent with THP-1 cell preparation, platelets were stained with 5 μM of carboxyfluorescein succinimidyl-ester (CFSE, Life Technologies) for 30 minutes at 37° C. To remove excess CFSE, stained platelets were pelleted in the presence of 10% ACD and 50 ng/ml of PGE1 and resuspended in Tyrode's buffer. Platelets were incubated with scIV.3, scIV.3-IdeS, or Tyrode's buffer (control) for 15 minutes at room temperature followed by the addition of anti-CD41 and anti-CD42b polyclonal rabbit antibodies (250 ng) or ITP sera (1:25 dilution). Antibody treated platelets (1×10⁷) were then added to THP-1 cells and incubated for 60 minutes at 37° C. Wells were washed twice with calcium-free PBS. THP-1 cells were then detached by the addition of 250 μL 0.05% trypsin/0.53% EDTA for 5 minutes at 37° C. Trypsin was quenched by the addition of 500 μL of culture media. Cells were stained with an antiplatelet antibody against CD42a to detect platelets adhered to the THP-1 cells, but not internalized. Cells were fixed in 2% formaldehyde and the samples were then analyzed by flow cytometry.

### Passive Model of ITP in Mice

Mice deficient in murine FcγRa receptors and expressing the full complement of human FcγR receptors and were intravenously (IV) injected via the tail vein with scIV.3-IdeS, or Tyrode's buffer (ref. A10: incorporated by reference in its entirety). After 30 minutes, a retro-orbital bleed was performed to count platelets and then mice were intraperitoneally (IP) injected with polyclonal rabbit-anti-mouse platelet serum (Cedarlanes; 5 mg/mL). Blood (50 uLs) was drawn via retro-orbital bleed at 2 and 24 hours post-antibody injection. A Hemavet 950FS was used to perform platelet counts. Whole blood was stained with mouse anti-CD45, anti-CD42a, anti-FcγRIIA, and Anti-His-Tag antibodies then and analyzed by flow cytometry to measure the cellular distribution of scIV.3-IdeS.

### Platelet Factor 4 (PF4)-Dependent P-Selectin Expression Assay

Platelets (6×10⁶) were incubated with PF4 (37.5 μg/mL) and either scIV.3-IdeS or Tyrode's (control) in a total volume of 40 μL for 30 minutes at room temperature. HIT patient serum (10 μL) was then mixed into each sample and incubated without agitation at room temperature. After 1 hour, platelets were stained with APC-conjugated anti-CD41 and FITC-conjugated anti-P-selectin antibodies for 10 minutes, fixed with 2% paraformaldehyde and analyzed by flow cytometry. The MFI of FITC-conjugated anti-P-selectin (CD62) of platelets (CD41+) was reported. In addition, as a positive control, platelets were stimulated with PAR1-activating) (Ojpeptide (AP) (NH2-SFLLRN; 25 μM) for 10 minutes in the presence of anti-CD41 and anti-P-selectin antibodies.

### Results

Characterization of the Binding Properties of the scIV.3

A single chain variable fragment of the monoclonal antibody, IV.3, a well-characterized high affinity antibody specific for human FcγRIIA, was generated by fusing the variable heavy (V_{H}) and variable light (V_{L}) chains with a flexible (GGGGS)₃ (SEQ ID NO: 123) linker. A sortag (LPET-SEQ ID NO: 124) was fused to the C-terminus with a short, semi-rigid linker, enabling efficient transpeptidation by the bacterial enzyme, sortase, and site-specific attachment of a FAM-containing peptide with minimal impact on target affinity (ref. A17; incorporated by reference in its entirety). scIV.3-LPETGG-FAM was incubated with platelets from wild-type (WT) mice, which lack FcγRIIA, or transgenic mice expressing human FcγRIIA (hFcγRIIA). As shown in FIG. 1, scIV.3-FAM bound to platelets expressing hFcγRIIA in a concentration-dependent manner, with a dissociation constant (K_{d}) of 0.27 nM. In contrast, there was no detectable binding of scIV.3-FAM to platelets from FcγRIIA-null mice (FIG. 1A). To determine the affinity that scIV.3 binds to human cells expressing FcγRIIA, increasing concentrations of scIV.3-FAM were incubated with THP-1 cells (a monocyte-like cell line), or platelets. The FAM-conjugated scIV.3 bound to both THP-1 cells and platelets with a high affinity, K_{d} 11 and 1 nM, respectively (FIG. 1B). Due to the approximately 100-fold difference in FcγRIIA expression between THP-1 (171,000±13,000 copies/cell) and platelets (1,000-5,000 copies/platelet) data are presented as percentage bound based on the MFI of the highest concentration of scIV.3-FAM tested, 40 nM (refs. A13, A18-A19; incorporated by reference in their entireties). To determine whether scIV.3 and full-length commercial IV.3 (cIV.3) had overlapping binding sites, platelets were treated with cIV.3 prior to staining with scIV.3-FAM. Incubation of platelets with cIV.3 prevented scIV.3-FAM from binding to platelets, as analyzed by fluorescent microscopy and flow cytometry (FIG. 1C-D).

Since neutrophils, monocytes, and platelets all express FcγRIIA, the cellular distribution of the scIV.3-FAM was determined in these populations in whole blood. Human whole blood was incubated with increasing concentrations of scIV.3-FAM, or cIV.3 (ref. A10; incorporated by reference in its entirety). The scIV.3-FAM bound to neutrophils, monocytes, and platelets in a dose-dependent manner (FIG. 1E). To provide further evidence that the binding of scIV.3 is functionally relevant, the ability of scIV.3 to block FcγRIIA-dependent platelet aggregation was examined in the presence of an anti-CD9 antibody. CD9 is a tetraspanin that is highly expressed (49,000±3,560 copies/platelet) on the surface of platelets. Although knowledge about CD9's precise physiologic function remains incomplete, anti-CD9 antibodies are known to cause platelet aggregation in a FcγRIIA-dependent manner (refs. A20-A22; incorporated by reference in their entireties). Tyrode's buffer (control) treated platelets exhibited dose-dependent aggregation in response to stimulation with anti-CD9 antibodies, with concentrations >1.25 μg/ml eliciting maximum aggregation. In contrast, platelets pre-treated with scIV.3 (50 nM) failed to respond to anti-CD9 antibodies at lower concentrations and had a >90% reduction in aggregation at the highest concentration tested (5 μg/ml) (FIG. 1F). Taken together, these data strongly indicate that scIV.3 retains the binding specificity and affinity for FcγRIIA on the surface of platelets.

Generation and Characterization of Recombinant scIV.3-IdeS

Recombinant IdeS with an N-terminal sortag (GGG) was produced, allowing a one-step, sortase-mediated reaction with scIV.3-LPETGG. This produced a single protein product, scIV.3-IdeS, which was purified from the reaction components using size exclusion HPLC. To determine if conjugation of IdeS to the c-terminus of scIV.3 affected its binding affinity for FcγRIIA, increasing concentrations of His-tagged scIV.3 or scIV.3-IdeS was incubated with human platelets (FIG. 2A). The scIV.3-IdeS fusion protein was found to have similar binding affinity (K_{d} 1.3 nM) to human platelets as scIV.3 (K_{d} 0.9 nM), and similar to scIV.3, the binding of scIV.3-IdeS to human platelets is also blocked by full-length cIV.3 (FIG. 2B). The ability of scIV.3-IdeS to degrade surface bound IgG is predicated on the recombinant protein being retained on the surface of cells; however, previous work has demonstrated engagement of FcγRIIA with divalent full-length IV.3 caused receptor internalization (ref. A15; incorporated by reference in its entirety). Experiments were conducted during development of embodiments herein to determine whether the monovalent ligand, scIV.3, causes receptor internalization when engaging FcγRIIA. The lowest concentration (5 nM) of His-tagged scIV.3-IdeS that caused full receptor occupancy on human platelets was incubated for up to 90 minutes. The level of surface bound scIV.3-IdeS was then measured by the binding of anti-His Tag antibodies via flow cytometry, and was found to be similar at all timepoints tested (FIG. 2C). These data indicate that FcγRIIA-bound scIV.3-IdeS remains on the surface of platelets for at least 90 minutes.

To test whether the fusion of IV.3 to the N-terminal of IdeS affected its enzymatic activity, human IgG (2 mg/mL) was incubated with increasing concentrations of either IdeS or scIV.3-IdeS. IdeS digests the heavy chains of IgG in a two-step process with the cleavage of the second heavy chain proceeding roughly 100-fold slower than the first (ref. A23: incorporated by reference in its entirety). The samples were then run on a 10% PAGE-gel under non-reducing conditions, and the formation of the Fc cleavage product of IgG was quantified by densitometry of the Coomassie Blue stained gel. Similar amounts of IgG were cleaved by IdeS and scIV.3-IdeS at 100 nM, resulting in mostly a single chain cleavage (FIG. 2D). Minimal IgG cleavage was observed at 10 or 1 nM by either IdeS or scIV.3-IdeS (FIG. 2D). The scIV.3 by itself was unable to cleave IgG (FIG. 2D). Taken together this data demonstrates that the fusion of scIV.3 and IdeS does not appreciably alter the binding affinity of the scFv, or the cleavage capacity of IdeS.

scIV.3-IdeS Inhibits IgG-Mediated Platelet Aggregation More Potently than scIV.3 Alone

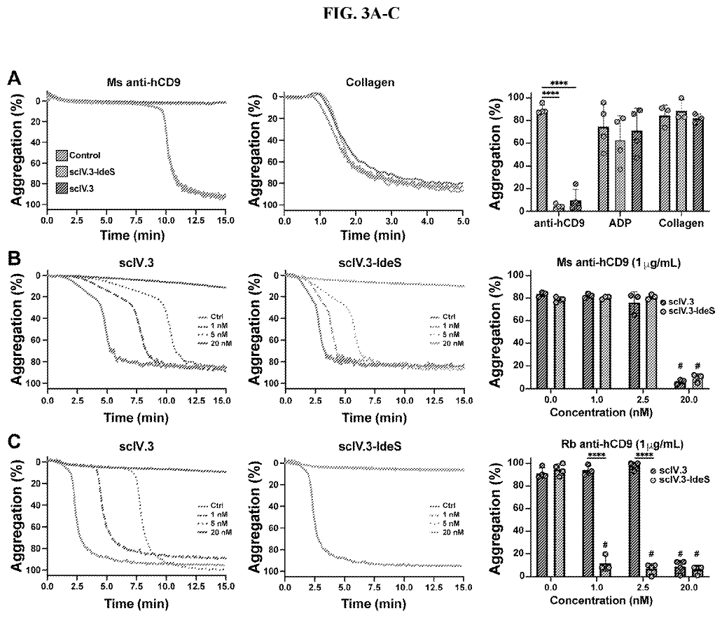

On platelets, IgG-dependent clustering of FcγRIIA causes platelet activation, but FcγRIIA has also been implicated in IgG-independent potentiation of integrin αIIbβ3 signaling (ref. A24-A25; incorporated by reference in their entireties). To examine whether scIV.3 or scIV.3-IdeS binding to FcγRIIA influences platelet sensitivity to agonists, platelets were stimulated with either ADP or collagen, two common platelet agonists that signal through the GPCRs (P2Y₁₂/P2Y₁) or GPVI and α2β1, respectively. To ensure the functional blocking of FcγRIIA, PRP incubated with scIV.3 or scIV.3-IdeS was stimulated with mouse anti-human CD9. The scIV.3 and scIV.3-IdeS, but not the control, inhibited anti-CD9 antibody mediated platelet aggregation (FIG. 3A). The binding of scIV.3 or scIV.3-IdeS to FcγRIIA did not inhibit platelet aggregation in response to collagen or ADP in PRP compared to vehicle control (FIG. 3A).

To determine whether surface bound scIV.3-IdeS can quickly neutralize IgG under biologically relevant conditions, the proteins were anchored to the platelet surface with sub-saturating concentrations of either and stimulated with commercial anti-CD9 IgG antibodies. The inhibitory effects of scIV.3 and scIV.3-IdeS on FcγRILA-dependent platelet activation were similar in platelets stimulated with mouse anti-human CD9, which cannot be cleaved by IdeS (FIG. 3B). When platelets were incubated with IdeS-cleavable monoclonal rabbit anti-human CD9, scIV.3-IdeS, but not scIV.3, was able to inhibit aggregation at 1 and 2.5 nM (FIG. 3C). Higher concentrations (20 nM) of either scIV.3 or scIV.3-IdeS were able to inhibit rabbit anti-human CD9 mediated platelet activation. scIV.3 and scIV.3-IdeS bind with similar affinity to FcγRIIA, but the conjugation of IdeS to scIV.3 enhanced its ability to prevent aggregation mediated by cleavable (rabbit) IgG, but not uncleavable (mouse) IgG.

Targeting scIV.3-IdeS to Platelet FcγRIIA Cleaves Antiplatelet Antibodies and Prevents Platelet Phagocytosis

IdeS helps streptococcal bacteria evade destruction by the human immune system, at least in part by the cleavage of opsonizing IgG bound to the surface of the bacteria. To examine whether scIV.3-IdeS targeted to the surface of platelets could neutralize antiplatelet IgG, scIV.3-IdeS-treated platelets were incubated with polyclonal rabbit IgG specific for human CD41 or CD42b, and then IgG cleavage and in vitro phagocytosis assays were performed. The amount of full-length rabbit anti-human CD41 and CD42b antibodies bound to the surface of platelets treated with scIV.3 (5 nM) was the same as platelets treated with vehicle control, as measured by flow cytometry with an anti-rabbit IgG heavy chain specific antibody (FIG. 4A). In contrast, platelets with scIV.3-IdeS (5 nM) on their surface had a decrease in the amount of heavy chain of either anti-CD41 or anti-CD42b antibodies (FIG. 4A). To determine how targeted therapy with scIV.3-IdeS influenced full-length antiplatelet IgG from binding platelets, parallel experiments were performed with equal concentrations of nontargeted IdeS (5 nM), as well as higher concentrations of IdeS (1000 nM), previously shown to cleave all IgG from a sample effectively. 5 nM of nontargeted IdeS was unable to cleave antiplatelet IgG. At the same time, higher concentrations of nontargeted IdeS were more efficient at reducing the amount of full-length anti-CD41 or anti-CD42b antibodies bound to the surface of platelets (FIG. 4A). To examine whether scIV.3-IdeS-coated platelets caused collateral IgG degradation, platelets coated with scIV.3-IdeS were incubated with equal volumes autologous platelet poor plasma. There was minimal detectable IgG cleavage product (~30 kDa fragment) at any of the scIV.3-IdeS-coated platelet concentrations tested (0.375 to 1.5×10⁸ platelets/mL) (FIG. 4B). An antibody-dependent in vitro phagocytosis assay was then performed to explore the physiological relevance of the reduction in heavy chain of antiplatelet associated antibodies with platelets. CFSE-stained platelets were incubated with a combination of CD41 and CD42b polyclonal antibodies in the presence of scIV.3, scIV.3-IdeS or control and then added to PMA-activated THP-1 cells. The number of THP-1 cells with surface adhered or internalized CFSE⁺ platelets was decreased in 15 platelets with surface-bound IdeS compared to platelets treated with scIV.3 or control (FIG. 4C). Furthermore, compared to platelets treated with scIV.3 or control, fewer scIV.3-IdeS treated platelets were phagocytosed by THP-1 cells (CFSE⁺/CD42a⁻) in the presence of antiplatelet antibodies (FIG. 4C). Mice with human FcγRIIA were IV injected with 10 μg of scIV.3-IdeS or buffer control, and 30 minutes later IP injected with 10 or 20 μg of rabbit anti-mouse platelet sera. After 24 hours, mice treated with scIV.3-IdeS had a higher platelet count than control treated mice. scIV.3-IdeS (FIG. 4D). The binding of scIV.3-IdeS to platelets in mice, was measured at 0.5, 2, and 24 hours post-injection. There was a significant increase in scIV.3-IdeS binding to platelets at 0.5 and 2 hours, but not 24 hours (FIG. 4E).

Platelets with Surface-Bound IdeS Neutralize the Fc-Dependent Effector Functions of ITP and

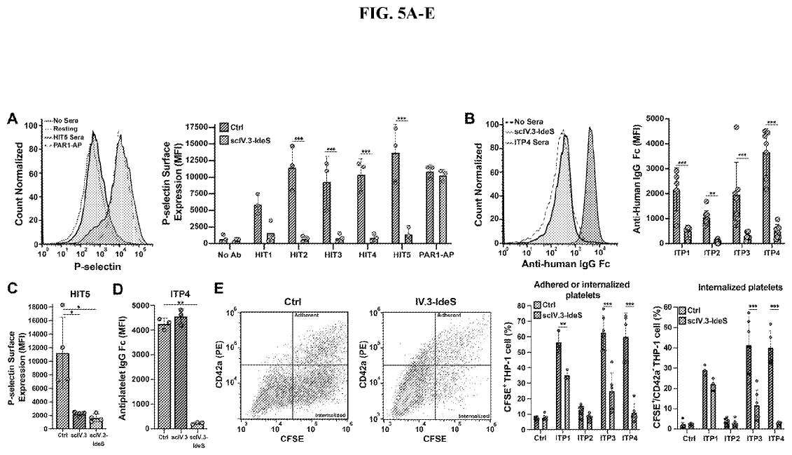

HIT Antibodies from Patient Sera Platelets with surface-bound IdeS efficiently cleaved and neutralized the Fc-dependent effector functions of commercial polyclonal antiplatelet antibodies. Experiments were conducted during development of embodiments herein to determine whether platelets with surface-bound IdeS neutralize the Fc-dependent effector functions of antibodies from HIT and ITP patients. Previously established PF4-dependent P-selectin surface expression assays were used to test the ability of scIV.3-IdeS to block HIT IgG-mediated FcγRIIA-dependent platelet activation (ref. A26; incorporated by reference in its entirety). Platelets treated with scIV.3-IdeS had decreased HIT IgG-mediated P-selectin surface expression compared to vehicle control-treated platelets (FIG. 5A). Platelets from healthy donors were incubated with sera from 4 patients with ITP, and the amount of intact antiplatelet antibodies remaining on the platelet surface following treatment with scIV.3-IdeS (5 nM) was quantified by flow cytometry using a mouse anti-human Fc specific antibody. The binding of full-length antiplatelet antibodies from the sera of all 4 patients was significantly reduced in platelets treated with scIV.3-IdeS compared to control (FIG. 5B). To examine whether blockage of FcγRIIA with the scIV.3 alone had an impact on HIT-IgG mediated platelet activation or prevented antiplatelet antibodies from binding platelets, respectively, platelets treated with scIV.3 or scIV.3-IdeS were exposed to HIT or ITP patient sera. Blocking FcγRIIA with scIV.3 inhibited HIT-IgG mediated platelet activation (FIG. 5C), but not the ability of antiplatelet IgG to bind to platelets (FIG. 5D). CFSE-stained platelets from healthy donors were incubated with antiplatelet antibodies from 4 separate ITP patients in the presence or absence of scIV.3-IdeS and incubated with THP-1 cells. Platelets treated with scIV.3-IdeS had a significant decrease in the number of platelets adherent to or internalized by activated THP-1 cells in 3 of the 4 ITP samples tested (FIG. 5E). Antibodies from ITP2 bound to platelets (FIG. 5B), but did not cause significant platelet phagocytosis. Finally, scIV.3-IdeS treated platelets significantly decreased antibody-mediated platelet phagocytosis in samples from two ITP donors (FIG. 5E). Taken together, this data demonstrates that platelet-targeted IdeS can neutralize the Fc-dependent effector functions of ITP and HIT antibodies from patient sera.

### Example 3

### Red Blood Cell Targeted IdeS

The destruction of erythrocytes by cell-bound IgGs (e.g., autoantibodies and/or alloantibodies) occurs in at least three clinically significant settings: warm Autoimmune Hemolytic Anemia (wAIHA), IgG-mediated hemolytic transfusion reaction (HTRs), and Hemolytic Disease of the Fetus and Newborn (HDFN). In each case, considerable effort is made to prevent exposure of red blood cells (RBCs) to antibody—e.g., via systemic immunosuppression in wAIHA, careful matching of transfused units in those with known alloantibodies, and immunoprophylaxis in RhD negative women. These strategies are not universally effective, however, and once acute and severe hemolysis has developed, it can be refractory to all available therapies. To address these issues, experiments were conducted during development of embodiments herein using IdeS fused to a series of single chain affinity ligands (scFv) which anchor the enzyme to the surface of murine or human red blood cells (RBCs).

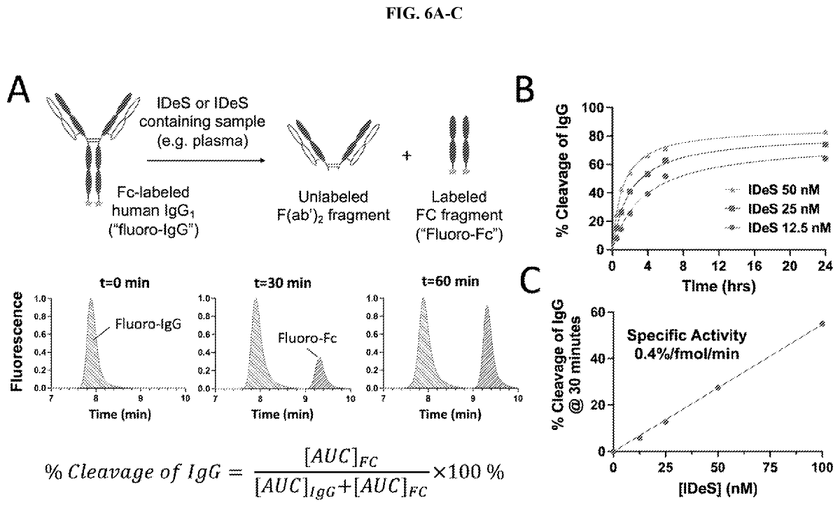

Experiments conducted during development of embodiments herein utilize two assays for measuring the activity of IdeS based therapies in vitro and in vivo. The first is an HPLC-based assay (FIG. 6), which measures cleavage of recombinant human IgG1 that has been site-specifically labeled with fluorophore at the C-terminus (i.e., on the Fc fragment). Using SEC HPLC, fluorescent signals from intact “fluoro-IgG” and cleaved “fluoro-Fc” can be completely separated and quantified, allowing precise calculation of product formation and reaction rate. The technique ultimately enables measurement of IdeS specific activity—e.g., allowing comparison of different lots of recombinant enzyme or quantification of IdeS activity in biological samples (e.g., plasma). The second assay involves injection of recombinant antibodies which have been modified to enable discrimination of intact vs. cleaved antibody via ELISA. Through simultaneous injection of RBC-binding antibody and non-binding control IgG, the technique measures the selectivity of erythrocyte-anchored IdeS for RBC-bound antibodies, one of the advantages over the untargeted enzyme.

Design, Synthesis, and Characterization of scFv-IdeS Fusion Protein

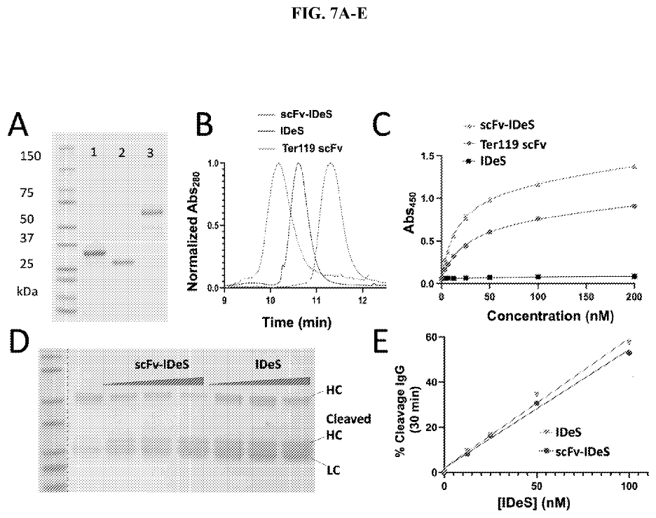

To anchor IdeS to the surface of murine erythrocytes, the enzyme was fused to Ter119 scFv, a widely reported single chain affinity ligand which binds Ly76, an antigen closely associated with the murine analogue of human glycophorin A (GPA) (Ref. B26-B27; incorporated by reference in their entireties). The resulting fusion protein was produced in a mammalian expression system (HEK293-6E cells) and purified in two steps: immobilized metal affinity chromatography (IMAC) and size exclusion HPLC (SEC). The fusion protein was relatively pure based on SDS-PAGE and analytical HPLC (FIGS. 7A and 7B) and was bound mouse RBCs with similar affinity (K_{d}~25 nm) to isolated Ter119 scFv (FIG. 7C). The IdeS activity of the fusion protein was similar to that of recombinant IdeS (FIGS. 7D and 7E).

Ter119 scFv-IdeS Cleaves RBC-Bound Antibodies

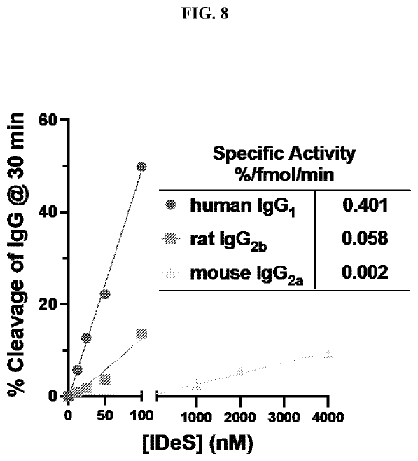

The capacity of the Ter119 scFv-IdeS fusion protein to cleave RBC-bound antibodies while bound to the erythrocyte surface was assessed. As a first example of an anti-RBC antibody, the Ter119 mAb itself was utilized, reasoning that the large antigen copy number (~106/RBC) would allow simultaneous loading of both mAb and fusion protein. Hybridoma-derived Ter119 mAb is a rat IgG2b, and while IdeS has been reported to have activity against this isotype (Ref. B19; incorporated by reference in its entirety), the HPLC-based assay revealed that the specific activity is actually ~8-fold lower than that seen with human IgG1 (FIG. 8). Consequently, a ‘humanized’ form of Ter119 mAb was engineered by replacing the rat IgG2b Fc fragment with that of human IgG1. The humanized Ter119 mAb expressed appropriately and was found to agglutinate mouse RBCs in the presence of an Fc-specific anti-human secondary F(ab′)₂ (an F(ab′)₂ was used to eliminate any possibility of IdeS cleaving the secondary antibody and disrupting agglutination in that manner.

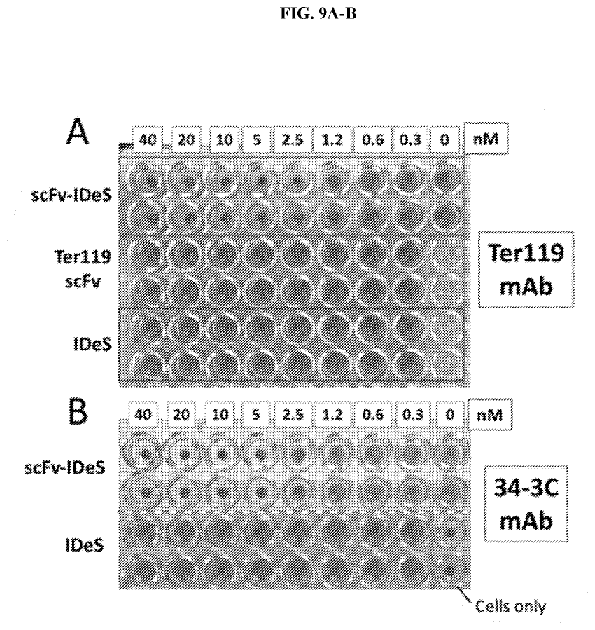

To measure cleavage of cell-bound antibody, Ter119 scFv-IdeS fusion protein and Ter119 mAb were loaded simultaneously onto mouse RBCs (on ice to prevent enzymatic cleavage) and washed to remove unbound protein. Isolated Ter119 scFv and untargeted IdeS were used as controls. As shown in FIG. 9A, the fusion protein—but not isolated Ter119 scFv or IdeS—inhibited agglutination at concentrations as low as 1.25 nM. The finding of functional effect well below the apparent K_{d} of the fusion protein indicates that a relatively small number of copies of surface-bound IdeS are sufficient to cleave the cell-bound mAb.

Experiments were conducted during development of embodiments herein to assess the capacity of Ter119 scFv-IdeS fusion protein to cleave antibody bound to the same protein (and epitope). T sequences of 34-3C were obtained from hybridomas derived from adult NZB mice, which develop spontaneous AIHA (Ref. B28; incorporated by reference in its entirety). As with Ter119 mAb, a humanized version of this clone was synthesized and agglutination of murine RBCs was confirmed (FIG. 9B). Again, Ter119 scFv-IdeS fusion protein—but not IdeS—inhibited agglutination, albeit with slightly less potency than that observed with Ter119 mAb.

### Erythrocyte Anchoring Extends Circulation Time of IdeS

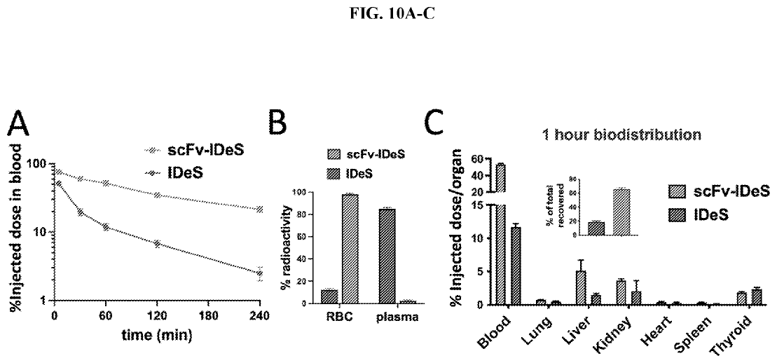

Experiments were conducted during development of embodiments herein to investigate the effects of erythrocyte anchoring on the blood PK of IdeS. Both IdeS and Ter119 scFv-IdeS fusion protein were radiolabeled with ¹²⁵I. Following confirmation of radiochemical purity (i.e., <2% free iodine), mice were intravenously injected with a 0.1 mg/kg dose of IdeS and an equimolar dose of fusion protein. As shown in FIG. 10A, IdeS cleared from the circulation quickly, with only ~10% of the injected dose remaining in the blood at 1 hour. In contrast, Ter119 scFv-IdeS demonstrated prolonged blood PK, with an ~4-fold increase in the AUC of the blood concentration vs. time curve. Moreover, the distribution of radioactivity within the blood was strikingly different, with nearly 100% of the scFv-IdeS signal found in the RBC pellet and >80% of the IdeS signal in the plasma (FIG. 10B). Organ biodistribution 1 hour after injection showed recovery of only ~20% of the injected dose of IdeS (FIG. 5C). A significant amount of IdeS radioactivity was found in the urine, suggesting rapid renal excretion.

Ter119 scFv-IdeS is More Potent than IdeS in a Mouse Model of Human IgG-Mediated Hemolysis

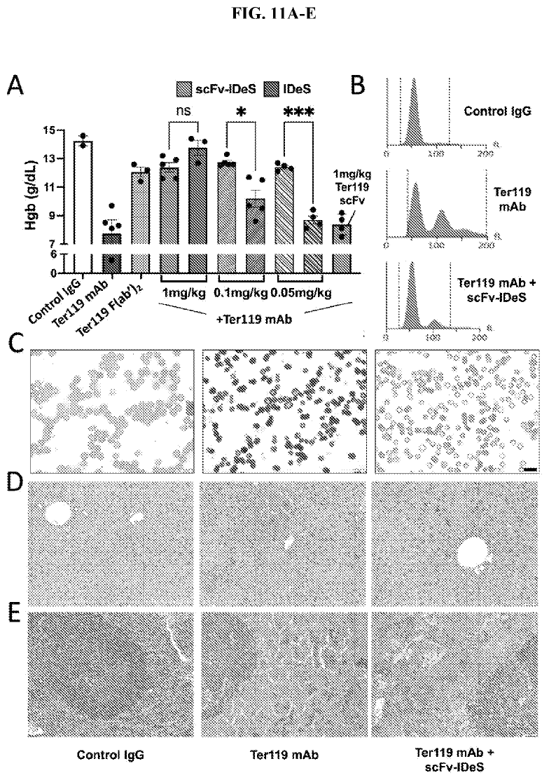

Having confirmed the cleavage of cell-bound antibodies in vitro and enhanced PK in vivo, experiments were conducted during development of embodiments herein to test the ability of Ter119 scFv-IdeS to cleave RBC-bound antibody in vivo and provide protection in a model of IgG-mediated hemolysis. Anti-RBC antibodies were engineered to have a murine Fc fragment to ensure proper interaction with Fc receptors. While IdeS has limited activity against mouse antibodies in general, reports have suggested that it cleaves mouse IgG2a efficiently (Ref. B29; incorporated by reference in its entirety). Using a quantitative activity assay, it was found the specific activity against mouse IgG2a was >200-fold less than human IgG1 (FIG. 8). Based results indicating strong interaction of human Fc with mouse Fc receptors, it was investigated whether a murine model of human IgG-mediated hemolysis could be created. Utilizing the ‘humanized’ Ter119 mAb, a range of antibody doses were injected and it was found that a single 2 mg/kg intravenous dose produced a significant drop in hemoglobin at 48 hours (7.7±0.9 g/dL, mean±SEM), as compared to a non-binding control (i.e., no target in mice) human IgG1 (14.2±0.5 g/dL). The drop in hemoglobin was confirmed to be the result of hemolysis and not impaired RBC production by the presence of marked reticulocytosis (FIG. 11B) and findings of hemolytic anemia on peripheral blood smear (anisocytosis and polychromasia, FIG. 11C).

To determine if enzymatic removal of the Fc fragment would impact the hemolysis observed in this model, Ter119 mAb was treated with IdeS ex vivo. The resulting Ter119 F(ab′)₂ was purified and injected at an equimolar dose. These mice also had a mild drop in hemoglobin (12.1±0.4 g/dL) as compared to the control IgG treated mice, although the result was also significantly different from the Ter119 mAb group (p<0.001). This result is consistent with at least one previous report indicating both Fc-dependent and independent mechanisms of hemolysis induced by Ter119 mAb (Ref. B31; incorporated by reference in its entirety).

With an established model of human IgG-mediated hemolysis, the protective effects of IdeS and Ter1 19 scFv-IdeS were tested, beginning with a relatively high dose (1 mg/kg IdeS or equimolar fusion protein). Both therapeutics were given 30 minutes before Ter119 mAb. With this dose and time interval, both treatments were highly effective, restoring the 48 hr hemoglobin to roughly the same level seen in Ter119 F(ab′)₂ treated mice (13.8±0.5 g/dL for IdeS and 12.4±0.3 for Ter119 scFv-IdeS, p=0.71). 0.1 and 0.05 mg/kg doses of IdeS (or equimolar fusion protein) were tested and significant differences between the two therapies were observed at each of these doses. Whereas the untargeted enzyme seemed to lose its protective effect on 48 hr hemoglobin (8.7±0.3 g/dL at 0.05 mg/kg), Ter1 19 scFv-IdeS fusion protein demonstrated nearly identical protection (12.4±0.2 g/dL, p<0.001 vs. IdeS).

Apart from its effect on hemoglobin, scFv-IdeS treatment largely reversed the reticulocytosis (FIG. 11B), anisocytosis, and polychromasia (FIG. 11C) observed in Ter119 mAb treated mice. Likewise, liver and spleen histopathology showed a significant reduction in extramedullary hematopoiesis (FIGS. 11D and 11E).

Ter119 scFv was also tested an important control. Given the massive reservoir of Ter119 antigen in the bloodstream (equivalent to a concentration of several micromolar), blocking the binding of Ter119 mAb is exceedingly unlikely (Ref. B24: incorporated by reference in its entirety). To explicitly test this possibility, however, mice were injected with 1 mg/kg of Ter119 scFv—a much larger dose than the 0.05 mg/kg Ter119 scFv-IdeS fusion—and no impact was found on hemolysis induced by a subsequent dose of 2 mg/kg Ter119 mAb (FIG. 11A).

### Example 4

### Pharmacokinetics of RBC-Targeted IdeS Constructs

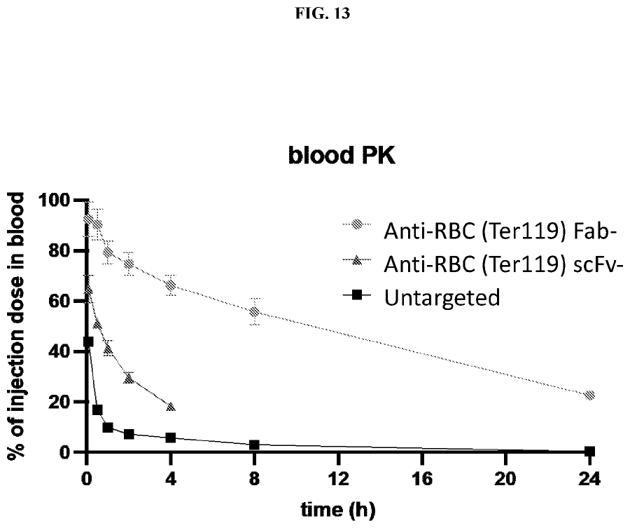

Experiments were conducted during development of embodiments herein to compare the pharmacokinetics of IdeS targeted to a RBC surface target (Ter119) by an ex3emplry Fab and scFv. FIG. 13 demonstrates that while both forms of RBC-targeted IdeS exhibit improved circulation times compared to the untargeted IdeS, the Fab-IdeS construct exhibits about a 4-fold to 5-fold improvement over the scFv IdeS construct.

### Example 5

### In Vitro Selectivity Assay

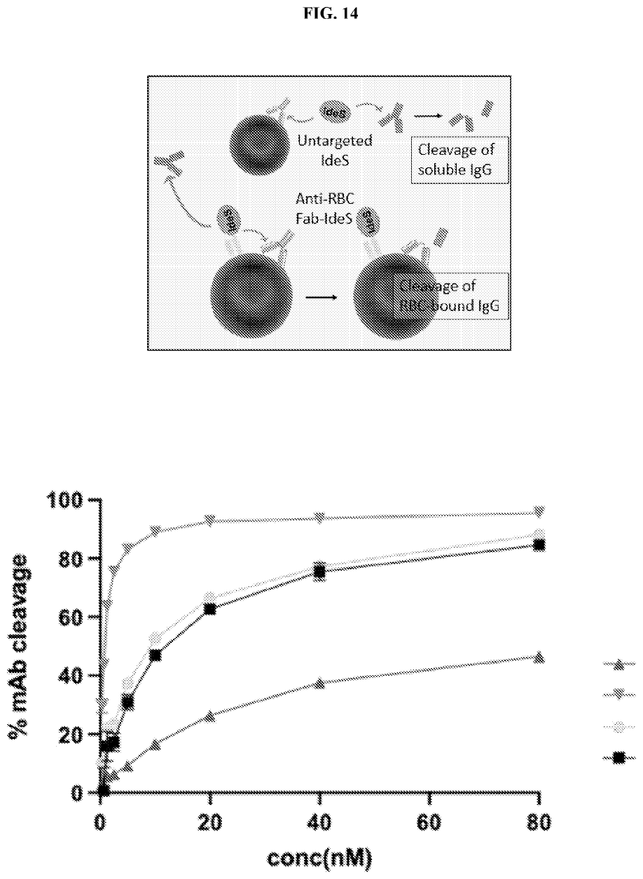

An in vitro selectivity assay was designed to compare the cleavage of RBC-bound IgG vs soluble IgG. Experiments conducted during development of embodiments herein demonstrate that untargeted IdeS cleaved RBC-bound and soluble IgG without preference, but IdeS targeted to RBC as part of a construct with an anti-RBC Fab exhibited a clear cleavage preference for the RBC-bound IgG (FIG. 14).

### Example 6

### Endothelial Cell Assay

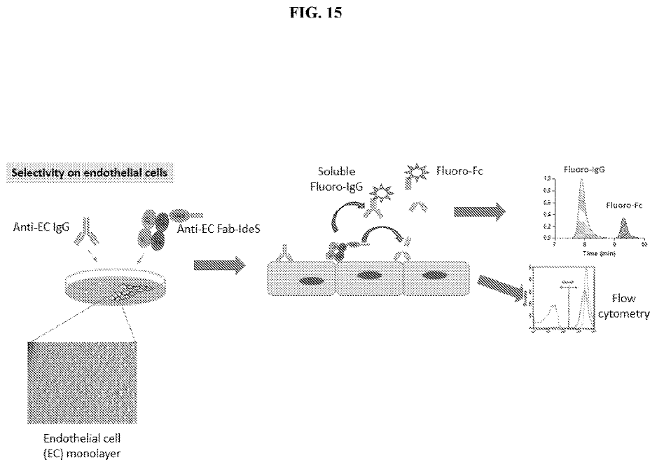

A monolayer of ECs is incubated with anti-EC IgG and anti-EC Fab-IdeS, then washed to remove unbound proteins. Fluorescently-tagged soluble IgG (Fluoro-IgG) is added, which does not bind ECs. Two readouts are be measured: (1) the cleavage of the soluble fluoro-IgG→Fluoro-Fc via HPLC, and (2) the amount of residual (i.e., uncleaved) EC-bound IgG via flow cytometry. The selectivity of the cell-bound Fab-IdeS is determined based on the relative cleavage of EC-bound and soluble IgGs (FIG. 15).

### Example 7

### In Vivo Selectivity

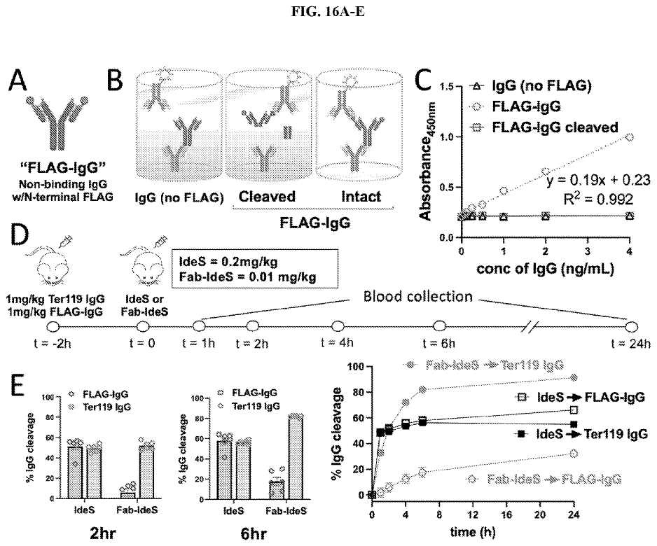

Erythrocyte-anchored IdeS selectively cleaves RBC-bound IgG in vivo To determine the selectivity of Ter119 Fab-IdeS for RBC-bound IgG in vivo, a novel assay was developed in which humanized Ter1 19 IgG is injected with an equal dose of N-terminal FLAG-tagged, non-RBC-binding, human IgG1 (“FLAG-IgG”). As shown in FIGS. 16B and 16C, intact vs. IdeS-cleaved FLAG-IgG can be quantified in mouse plasma using a sandwich ELISA, enabling calculation of the % cleavage of non-RBC bound IgG. Mice were injected with both antibodies and, two hours later, given a 0.2 mg/kg dose of IdeS or 0.01 mg/kg dose of Fab-IdeS (FIG. 1D). Blood was collected at the indicated times and separated into plasma for sandwich ELISA and RBCs for flow cytometry with an Fc-specific antibody to determine the amount of intact RBC-bound IgG. The % cleavage of RBC-bound antibody was calculated by normalizing the flow signal to untreated mice. Of note, a 20-fold difference in dose (40-fold in terms of molar equivalents of IdeS) was required to bring the % cleavage of RBC-bound IgG into the same range, reflecting the much higher potency of Ter119 Fab-IdeS as compared to its soluble counterpart. FIG. 16E also shows major differences in the behavior of these two drugs in vivo: 1. IdeS cleaves soluble and RBC-bound IgGs equally, whereas Ter119 Fab-IdeS has ~5-fold selectivity for RBC-bound IgG, 2. Nearly all IgG cleavage by IdeS occurs in the 1st hour after administration, whereas Ter119-Fab IdeS continues to have activity 24 hours later.

### Example 8

### Immunogenicity

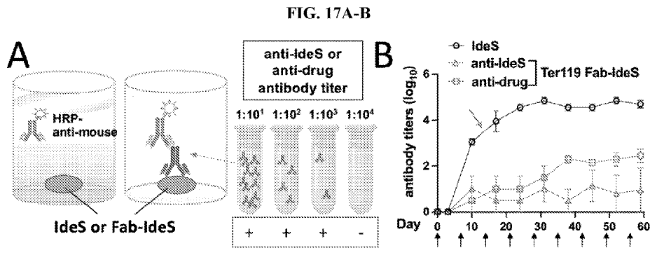

Ter119 Fab-IdeS results in lower levels of anti-drug antibodies than untargeted IdeS following repeated injection in healthy mice. The humoral immune response to weekly, intravenous doses of 12.5 μg of Ter1 19 Fab-IdeS vs. equimolar doses of untargeted IdeS was evaluated. Plasma was collected 3 days after each injection and antibody titers were quantified using sandwich ELISA (FIG. 17A). Mice injected with IdeS were given just three doses, as titers increased >4 orders of magnitude and most animals suffered acute anaphylaxis with additional doses. In contrast, mice tolerated nine weekly doses of Ter119 Fab-IdeS, maintaining low anti-IdeS titers throughout (FIG. 17B). A separate ELISA was performed using Ter119 Fab-IdeS as the capture molecule to determine ADA titer. This revealed slightly higher antibody titers in Ter119 Fab-IdeS treated mice, indicating some level of immunogenicity to non-IdeS components of the fusion protein—e.g., the rat-derived Fab fragment (FIG. 17B).

### REFERENCES

The following references, some of which are cited above, are incorporated by reference in their entireties.
A1. G. Vidarsson, G. Dekkers, T. Rispens, IgG subclasses and allotypes: from structure to effector functions. Frontiers in immunology 5, 520 (2014).
A2. S. M. Hayter, M. C. Cook, Updated assessment of the prevalence, spectrum and case definition of autoimmune disease. Autoimmunity reviews 11, 754-765 (2012).
A3. S. Mithoowani, D. M. Arnold, First-line therapy for immune thrombocytopenia. Hämostaseologie 39, 259-265 (2019).
A4 D. Provan et al., Updated international consensus report on the investigation and management of primary immune thrombocytopenia. Blood advances 3, 3780-3817 (2019).
A5. U. von Pawel-Rammingen, B. P. Johansson, L. Björck, IdeS, a novel streptococcal cysteine proteinase with unique specificity for immunoglobulin G. The EMBO journal 21, 1607-1615 (2002).
A6 C. Kizlik-Masson et al., Cleavage of anti-PF4/heparin IgG by a bacterial protease and potential benefit in heparin-induced thrombocytopenia. blood 133, 2427-2435 (2019).
A7. B. P. Johansson, O. Shannon, L. Björck, IdeS: a bacterial proteolytic enzyme with therapeutic potential. PLOS one 3, e1692 (2008).
A8. L. Winstedt et al., Complete removal of extracellular IgG antibodies in a randomized dose-escalation phase I study with the bacterial enzyme IdeS-a novel therapeutic opportunity. PLOS One 10, e0132011 (2015).
A9. J. I. Shin, D. Geetha, W. M. Szpirt, M. Windpessl, A. Kronbichler, Anti-Glomerular Basement Membrane Disease (Goodpasture Disease): From Pathogenesis to Plasma Exchange to IdeS. Therapeutic Apheresis and Dialysis, (2021).
A10. P. Smith, D. J. DiLillo, S. Bournazos, F. Li, J. V. Ravetch, Mouse model recapitulating human Fcγ receptor structural and functional diversity. Proceedings of the National Academy of Sciences 109, 6181-6186 (2012).
A11. A. R. Crow, A. H. Lazarus, Role of Fcγ receptors in the pathogenesis and treatment of idiopathic thrombocytopenia purpura. Journal of pediatric hematology/oncology 25, S14-S18 (2003).
A12. M. U. Ahmed et al., Respective roles of Glycoprotein VI and FcγRIIA in the regulation of αIIbβ3-mediated platelet activation to fibrinogen, thrombus buildup, and stability. Research and practice in thrombosis and haemostasis 5, e12551 (2021).
A13. P. Patel, J. V. Michael, U. P. Naik, S. E. Mckenzie, Platelet FcγRIIA in immunity and thrombosis: Adaptive immunothrombosis. Journal of Thrombosis and Haemostasis 19, 1149-1160 (2021).
A14. R. Looney, G. Abraham, C. L. Anderson, Human monocytes and U937 cells bear two distinct Fc receptors for IgG. The Journal of Immunology 136, 1641-1647 (1986).
A15. B. Chen et al., Humanised effector-null FcγRIIA antibody inhibits immune complex-mediated proinflammatory responses. Annals of the rheumatic diseases 78, 228-237 (2019).
A16. C. T. Sardjono, B. Wines, M. Powel, M. Hogarth, Epitope Mapping of Fc gamma RIIa Monoclonal Antibodies. Indonesian Journal of Biotechnology 13, (2008).
A17. C. F. Greineder et al., Site-specific modification of single-chain antibody fragments for bioconjugation and vascular immunotargeting. Bioconjugate chemistry 29, 56-66 (2018).
A18. M. E. Ackerman et al., A robust, high-throughput assay to determine the phagocytic activity of clinical antibody samples. Journal of immunological methods 366, 8-19 (2011).
A19. J. Qiao, M. Al-Tamimi, R. I. Baker, R. K. Andrews, E. E. Gardiner, The platelet Fc receptor, FcγRIIa. Immunological reviews 268, 241-252 (2015).
A20. M. B. Protty et al., Identification of Tspan9 as a novel platelet tetraspanin and the collagen receptor GPVI as a component of tetraspanin microdomains. Biochemical Journal 417, 391-401 (2009).
A21. P. Horsewood, C. Hayward, T. E. Warkentin, J. G. Kelton, Investigation of the mechanisms of monoclonal antibody-induced platelet activation. (1991).
A22. R. Qi et al., Differential activation of human platelets induced by Fc gamma receptor II cross-linking and by anti-CD9 monoclonal antibody. The Journal of Immunology 157, 5638-5645 (1996).
A23. R. Vindebro, C. Spoerry, U. von Pawel-Rammingen, Rapid IgG heavy chain cleavage by the streptococcal IgG endopeptidase IdeS is mediated by IdeS monomers and is not due to enzyme dimerization. FEBS letters 587, 1818-1822 (2013).
A24. B. Boylan et al., Identification of FcγRIIa as the ITAM-bearing receptor mediating αIIbβ3 outside-in integrin signaling in human platelets. Blood, The Journal of the American Society of Hematology 112, 2780-2786 (2008).
A25. H. Zhi et al., Cooperative integrin/ITAM signaling in platelets enhances thrombus formation in vitro and in vivo. Blood, The Journal of the American Society of Hematology 121, 1858-1867 (2013).
A. Padmanabhan et al., A novel PF4-dependent platelet activation assay identifies A26. patients likely to have heparin-induced thrombocytopenia/thrombosis. Chest 150, 506-515 (2016).
A27. M. Swinkels et al., Emerging concepts in immune thrombocytopenia. Frontiers in immunology 9, 880 (2018).
A28. H. Al-Samkari et al., A modern reassessment of glycoprotein-specific direct platelet autoantibody testing in immune thrombocytopenia. Blood advances 4, 9-18 (2020).
A29. S. M. Hicks, L. A. Coupland, A. Jahangiri, P. Y. Choi, E. E. Gardiner, Novel scientific approaches and future research directions in understanding ITP. Platelets 31, 315-321 (2020).
A30. M. J. Stubbs et al., Administration of immunoglobulin G-degrading enzyme of Streptococcus pyogenes (IdeS) for persistent anti-ADAMTS13 antibodies in patients with thrombotic thrombocytopenia purpura in clinical remission. British journal of haematology 186, 137-140 (2019).
A31. B. E. Lonze, A review of imlifidase in solid organ transplantation. Expert Opinion on Biological Therapy 21, 135-143 (2021).
A32. N. Cloutier et al., Platelets release pathogenic serotonin and return to circulation after immune complex-mediated sequestration. Proceedings of the National Academy of Sciences 115, E1550-E1559 (2018).
A33. I. Melki et al., Platelets release mitochondrial antigens in systemic lupus erythematosus. Science Translational Medicine 13, (2021).
A34. J. Perdomo et al., Neutrophil activation and NETosis are the major drivers of thrombosis in heparin-induced thrombocytopenia. Nature communications 10, 1-14 (2019).
A35. B. Zhang, S. M. Vidanapathirana, C. F. Greineder, in Peptide Conjugation. (Springer, 2021), pp. 163-173.
B1. Brodsky R A. Warm Autoimmune Hemolytic Anemia. N Engl J Med. 2019; 381 (7): 647-54. Epub 2019 Aug. 15. doi: 10.1056/NEJMcp1900554. PubMed PMID: 31412178.
B2. Panch S R, Montemayor-Garcia C, Klein H G. Hemolytic Transfusion Reactions. N Engl J Med. 2019; 381 (2): 150-62. Epub 2019 Jul. 11. doi: 10.1056/NEJMra1802338. PubMed PMID: 31291517.
B3. Delaney M, Matthews D C. Hemolytic disease of the fetus and newborn: managing the mother, fetus, and newborn. Hematology Am Soc Hematol Educ Program. 2015; 2015:146-51. Epub 2015 Dec. 8. doi: 10.1182/asheducation-2015.1.146. PubMed PMID: 26637714.
B4. Thein S L, Pirenne F, Fasano R M, Habibi A, Bartolucci P, Chonat S, Hendrickson J E, Stowell S R. Hemolytic transfusion reactions in sickle cell disease: underappreciated and potentially fatal. Haematologica. 2020; 105 (3): 539-44. Epub 2020 Feb. 8. doi: 10.3324/haematol.2019.224709. PubMed PMID: 32029505; PMCID: PMC7049330.
B5. Fattizzo B, Zaninoni A, Nesa F, Sciumbata V M, Zanella A, Cortelezzi A, Barcellini W. Lessons from very severe, refractory, and fatal primary autoimmune hemolytic anemias. Am J Hematol. 2015; 90 (8): E149-51. Epub 2015 May 13. doi: 10.1002/ajh.24047. PubMed PMID: 25963403.
B6. Lafarge A, Bertinchamp R, Pichereau C, Valade S, Chermak A, Theodose I, Canet E, Lemiale V, Schlemmer B, Galicier L, Azoulay E, Mariotte E. Prognosis of autoimmune hemolytic anemia in critically ill patients. Ann Hematol. 2019; 98 (3): 589-94. Epub 2018 Dec. 12. doi: 10.1007/s00277-018-3553-9. PubMed PMID: 30536106.
B7. Barcellini W, Zaninoni A, Fattizzo B, Giannotta J A, Lunghi M, Ferrari A, Leporace A P, Maschio N, Scaramucci L, Cantoni S, Chiurazzi F, Consonni D, Rossi G, De Fabritiis P, Gaidano G, Zanella A, Cortelezzi A. Predictors of refractoriness to therapy and healthcare resource utilization in 378 patients with primary autoimmune hemolytic anemia from eight Italian reference centers. Am J Hematol. 2018; 93 (9): E243-e6. Epub 2018 Jul. 8. doi: 10.1002/ajh.25212. PubMed PMID: 29981267.
B8. Barcellini W, Fattizzo B. How I treat warm autoimmune hemolytic anemia. Blood. 2021; 137 (10): 1283-94. Epub 2021 Jan. 30. doi: 10.1182/blood.2019003808. PubMed PMID: 33512406.
B9. Delaney M, Wendel S, Bercovitz R S, Cid J, Cohn C, Dunbar N M, Apelseth T O, Popovsky M, Stanworth S J, Tinmouth A, Van De Watering L, Waters J H, Yazer M, Ziman A.
Transfusion reactions: prevention, diagnosis, and treatment. Lancet. 2016; 388 (10061): 2825-36. Epub 2016 Apr. 17. doi: 10.1016/s0140-6736 (15) 01313-6. PubMed PMID: 27083327.
B10. Gardner K, Hoppe C, Mijovic A, Thein S L. How we treat delayed haemolytic transfusion reactions in patients with sickle cell disease. Br J Haematol. 2015; 170 (6): 745-56. Epub 2015 May 15. doi: 10.1111/bjh.13494. PubMed PMID: 25967919.
B11. Jager U, Barcellini W, Broome C M, Gertz M A, Hill A, Hill Q A, Jilma B, Kuter D J, Michel M, Montillo M, Roth A, Zeerleder S S, Berentsen S. Diagnosis and treatment of autoimmune hemolytic anemia in adults: Recommendations from the First International Consensus Meeting. Blood Rev. 2020; 41:100648. Epub 2019 Dec. 17. doi: 10.1016/j.blre.2019.100648. PubMed PMID: 31839434.
B12. Ruivard M, Tournilhac O, Montel S, Fouilhoux A C, Quainon F, Lenat A, Travade P, Philippe P. Plasma exchanges do not increase red blood cell transfusion efficiency in severe autoimmune hemolytic anemia: a retrospective case-control study. J Clin Apher. 2006; 21 (3): 202-6. Epub 2006 Apr. 12. doi: 10.1002/jca.20096. PubMed PMID: 16607631.
B13. Deng J, Zhou F, Wong C Y, Huang E, Zheng E. Efficacy of therapeutic plasma exchange for treatment of autoimmune hemolytic anemia: A systematic review and meta-analysis of randomized controlled trials. J Clin Apher. 2020; 35 (4): 294-306. Epub 2020 May 10. doi: 10.1002/jca.21790. PubMed PMID: 32384203.
B14. Galeotti C, Kaveri S V, Bayry J. IVIG-mediated effector functions in autoimmune and inflammatory diseases. Int Immunol. 2017; 29 (11): 491-8. Epub 2017 Jul. 2. doi: 10.1093/intimm/dxx039. PubMed PMID: 28666326.
B15. Schwab I, Nimmerjahn F. Intravenous immunoglobulin therapy: how does IgG modulate the immune system? Nat Rev Immunol. 2013; 13 (3): 176-89. Epub 2013 Feb. 16. doi: 10.1038/nri3401. PubMed PMID: 23411799.
B16. Qureshi O S, Rowley T F, Junker F, Peters S J, Crilly S, Compson J, Eddleston A, Bjorkelund H, Greenslade K, Parkinson M, Davies N L, Griffin R, Pither T L, Cain K, Christodoulou L, Staelens L, Ward E, Tibbitts J, Kiessling A, Smith B, Brennan F R, Malmqvist M, Fallah-Arani F, Humphreys D P. Multivalent Fc y-receptor engagement by a hexameric Fc-fusion protein triggers Fc y-receptor internalisation and modulation of Fc y-receptor functions. Sci Rep. 2017; 7 (1): 17049. Epub 2017 Dec. 8. doi: 10.1038/s41598-017-7255-8. PubMed PMID: 29213127;
B17. Bussel J, Arnold D M, Grossbard E, Mayer J, Treliński J, Homenda W, Hellmann A,
Windyga J, Sivcheva L, Khalafallah A A, Zaja F, Cooper N, Markovtsov V, Zayed H, Duliege A M. Fostamatinib for the treatment of adult persistent and chronic immune thrombocytopenia: Results of two phase 3, randomized, placebo-controlled trials. Am J Hematol. 2018; 93 (7): 921-30. Epub 2018 Apr. 27. doi: 10.1002/ajh.25125. PubMed PMID: 29696684; PMCID: PMC6055608.
B18. Winstedt L, Jarnum S, Nordahl E A, Olsson A, Runstrom A, Bockermann R, Karlsson C, Malmstrom J, Palmgren G S, Malmqvist U, Bjorck L, Kjellman C. Complete Removal of Extracellular IgG Antibodies in a Randomized Dose-Escalation Phase I Study with the Bacterial Enzyme IdeS—A Novel Therapeutic Opportunity. PLOS One. 2015; 10 (7): e0132011. Epub 2015 Jul. 16. doi: 10.1371/journal.pone.0132011. PubMed PMID: 26177518; PMCID:
B19. Johansson B P, Shannon O, Bjorck L. IdeS: a bacterial proteolytic enzyme with therapeutic potential. PLOS One. 2008; 3 (2): e1692. Epub 2008 Feb. 28. doi: 10.1371/journal.pone.0001692. PubMed PMID: 18301769; PMCID: PMC2253494
B20. Jordan S C, Lorant T, Choi J, Kjellman C, Winstedt L, Bengtsson M, Zhang X, Eich T, Toyoda M, Eriksson B M, Ge S, Peng A, Jarnum S, Wood K J, Lundgren T, Wennberg L, Backman L, Larsson E, Villicana R, Kahwaji J, Louie S, Kang A, Haas M, Nast C, Vo A, Tufveson G. IgG Endopeptidase in Highly Sensitized Patients Undergoing Transplantation. N Engl J Med. 2017; 377 (5): 442-53. Epub 2017 Aug. 3. doi: 10.1056/NEJMoa1612567. PubMed PMID: 28767349.
B21. Shin J I, Geetha D, Szpirt W M, Windpessl M, Kronbichler A. Anti-glomerular basement membrane disease (Goodpasture disease): From pathogenesis to plasma exchange to IdeS. Ther Apher Dial. 2021. Epub 2021 Aug. 3. doi: 10.1111/1744-9987.13718. PubMed PMID: 34339589.
B22. Takahashi R, Yuki N. Streptococcal IdeS: therapeutic potential for Guillain-Barre syndrome. Sci Rep. 2015; 5:10809. Epub 2015 Jul. 22. doi: 10.1038/srep10809. PubMed PMID: 26194472; PMCID: PMC4508529 member of Expert Review of Neurotherapeutics, The Journal of the Neurological Sciences, The Journal of Peripheral Nervous System, Neurosurgery & Psychiatry.
B23. Chessa L, Leuzzi V, Plebani A, Soresina A, Micheli R, D′Agnano D, Venturi T, Molinaro A, Fazzi E, Marini M, Ferremi Leali P, Quinti I, Cavaliere F M, Girelli G, Pietrogrande M C, Finocchi A, Tabolli S, Abeni D, Magnani M. Intra-erythrocyte infusion of dexamethasone reduces neurological symptoms in ataxia teleangiectasia patients: results of a phase 2 trial. Orphanet J Rare Dis. 2014; 9:5. Epub 2014 Jan. 11. doi: 10.1186/1750-1172 Sep. 5. PubMed PMID: 24405665; PMCID: PMC3904207.
B24. Villa C H, Cines D B, Siegel D L, Muzykantov V. Erythrocytes as Carriers for Drug Delivery in Blood Transfusion and Beyond. Transfus Med Rev. 2017; 31 (1): 26-35. Epub 2016 Oct. 7. doi: 10.1016/j.tmrv.2016.08.004. PubMed PMID: 27707522; PMCID: PMC5161683.
B25. Villa C H, Pan D C, Johnston I H, Greineder C F, Walsh L R, Hood E D, Cines D B, Poncz M, Siegel D L, Muzykantov V R. Biocompatible coupling of therapeutic fusion proteins to human erythrocytes. Blood Adv. 2018; 2 (3): 165-76. Epub 2018 Jan. 25. doi: 10.1182/bloodadvances.2017011734. PubMed PMID: 29365311; PMCID: PMC5812327 Anokion S A. V.R.M., D.L.S., and C.H.V.
B26. Kina T, Ikuta K, Takayama E, Wada K, Majumdar A S, Weissman I L, Katsura Y. The monoclonal antibody TER-119 recognizes a molecule associated with glycophorin A and specifically marks the late stages of murine erythroid lineage. Br J Haematol. 2000; 109 (2): 280-7. Epub 2000 Jun. 10. doi: 10.1046/j.1365-2141.2000.02037.x. PubMed PMID: 10848813.
B27. Greineder C F, Howard M D, Carnemolla R, Cines D B, Muzykantov V R. Advanced drug delivery systems for antithrombotic agents. Blood. 2013; 122 (9): 1565-75. Epub 2013 Jun. 27. doi: 10.1182/blood-2013-03-453498. PubMed PMID: 23798715; PMCID: PMC3757370.
B28. Shibata T, Berney T, Reininger L, Chicheportiche Y, Ozaki S, Shirai T, Izui S. Monoclonal antierythrocyte autoantibodies derived from NZB mice cause autoimmune hemolytic anemia by two distinct pathogenic mechanisms. Int Immunol. 1990; 2 (12): 1133-41. Epub 1990 Jan. 1. doi: 10.1093/intimm/2.12.1133. PubMed PMID: 2090198.
B29. Nandakumar K S, Johansson B P, Bjorck L, Holmdahl R. Blocking of experimental arthritis by cleavage of IgG antibodies in vivo. Arthritis Rheum. 2007; 56 (10): 3253-60. Epub 2007 Oct. 2. doi: 10.1002/art.22930. PubMed PMID: 17907170.
B30. Dekkers G, Bentlage AEH, Stegmann T C, Howie H L, Lissenberg-Thunnissen S, Zimring J, Rispens T, Vidarsson G. Affinity of human IgG subclasses to mouse Fc gamma receptors. MAbs. 2017; 9 (5): 767-73. Epub 2017 May 4. doi: 10.1080/19420862.2017.1323159. PubMed PMID: 28463043; PMCID: PMC5524164.
B31. Mqadmi A, Abramowitz S, Zheng X, Yazdanbakhsh K. Reduced red blood cell destruction by antibody fragments. Immunohematology. 2006; 22 (1): 11-4. Epub 2006 Mar. 28. PubMed PMID: 16563046; PMCID: PMC4797626.
B32. Faul F, Erdfelder E, Buchner A, Lang A G. Statistical power analyses using G*Power 3.1: tests for correlation and regression analyses. Behav Res Methods. 2009; 41 (4): 1149-60. Epub 2009 Nov. 10. doi: 10.3758/BRM.41.4.1149. PubMed PMID: 19897823.
B33. Faul F, Erdfelder E, Lang A G, Buchner A. G*Power 3: a flexible statistical power analysis program for the social, behavioral, and biomedical sciences. Behav Res Methods. 2007; 39 (2): 175-91. Epub 2007 Aug. 19. doi: 10.3758/bf03193146. PubMed PMID: 17695343.
B34. Hod E A, Cadwell C M, Liepkalns J S, Zimring J C, Sokol S A, Schirmer D A, Jhang J, Spitalnik S L. Cytokine storm in a mouse model of IgG-mediated hemolytic transfusion reactions. Blood. 2008; 112 (3): 891-4. Epub 2008 May 17. doi: 10.1182/blood-2008-01-132092. PubMed PMID: 18483395; PMCID: PMC2481546.
B35. Hod E A. Consequences of hemolysis: Pro-inflammatory cytokine response to erythrophagocytosis. Transfus Clin Biol. 2019; 26 (2): 125-7. Epub 2019 Mar. 14. doi: 10.1016/j.tracli.2019.02.005. PubMed PMID: 30862396.
B36. Kosanke J. EDTA glycine acid treatment of red blood cells. Immunohematology. 2012; 28 (3): 95-6. Epub 2013 Jan. 5. PubMed PMID: 23286556.
B37. Burin des Roziers N, Squalli S. Removing IgG antibodies from intact red cells: comparison of acid and EDTA, heat, and chloroquine elution methods. Transfusion. 1997; 37 (5): 497-501. Epub 1997 May 1. doi: 10.1046/j. 1537-2995.1997.37597293880.x. PubMed PMID: 9149774.
B38. Sheiner L B, Steimer J L. Pharmacokinetic/pharmacodynamic modeling in drug development. AnnuRev Pharmacol Toxicol. 2000; 40:67-95. Epub 2000 Jun. 3. doi: 10.1146/annurev.pharmtox.40.1.67. PubMed PMID: 10836128.
B39. Kontos S, Kourtis I C, Dane K Y, Hubbell J A. Engineering antigens for in situ erythrocyte binding induces T-cell deletion. Proc Natl Acad Sci USA. 2013; 110 (1): E60-8. Epub 2012 Dec. 19.
B40. Lorentz K M, Kontos S, Diaceri G, Henry H, Hubbell J A. Engineered binding to erythrocytes induces immunological tolerance to E. coli asparaginase. Sci Adv. 2015; 1 (6): e1500112. Epub 2015 Nov. 26. doi: 10.1126/sciadv.1500112. PubMed PMID: 26601215; PMCID: PMC4646778.
B41. Ermert D, Weckel A, Magda M, Morgelin M, Shaughnessy J, Rice P A, Bjorck L, Ram S, Blom A M. Human IgG Increases Virulence of Streptococcus pyogenes through Complement Evasion. J Immunol. 2018; 200 (10): 3495-505. Epub 2018 Apr. 8. doi: 10.4049/jimmunol.1800090. PubMed PMID: 29626087; PMCID: PMC6028238.
B42. Vindebro R, Spoerry C, von Pawel-Rammingen U. Rapid IgG heavy chain cleavage by the streptococcal IgG endopeptidase IdeS is mediated by IdeS monomers and is not due to enzyme dimerization. FEBS Lett. 2013; 587 (12): 1818-22. Epub 2013 May 15. doi: 10.1016/j.febslet.2013.04.039. PubMed PMID: 23665032.
B43. Akesson P, Moritz L, Truedsson M, Christensson B, von Pawel-Rammingen U. IdeS, a highly specific immunoglobulin G (IgG)-cleaving enzyme from Streptococcus pyogenes, is inhibited by specific IgG antibodies generated during infection. Infect Immun. 2006; 74 (1): 497-503. Epub 2005 Dec. 22. doi: 10.1128/iai.74.1.497-503.2006. PubMed PMID: 16369006; PMCID: PMC1346671.
B44. Ayalon G, Lee S H, Adolfsson O, Foo-Atkins C, Atwal J K, Blendstrup M, Booler H, Bravo J, Brendza R, Brunstein F, Chan R, Chandra P, Couch J A, Datwani A, Demeule B, DiCara D, Erickson R, Ernst J A, Foreman O, He D, Hotzel I, Keeley M, Kwok MCM, Lafrance-Vanasse J, Lin H, Lu Y, Luk W, Manser P, Muhs A, Ngu H, Pfeifer A, Pihlgren M, Rao G K, Scearce-Levie K, Schauer S P, Smith W B, Solanoy H, Teng E, Wildsmith K R, Bumbaca Yadav D, Ying Y, Fuji R N, Kerchner G A. Antibody semorinemab reduces tau pathology in a transgenic mouse model and engages tau in patients with Alzheimer's disease. Sci Transl Med. 2021; 13 (593). Epub 2021 May 14. doi: 10.1126/scitranslmed.abb2639. PubMed PMID: 33980574.
B45. Kiseleva R Y, Glassman P G, LeForte K M, Walsh L R, Villa C H, Shuvaev V V, Myerson J W, Aprelev P A, Marcos-Contreras O A, Muzykantov V R, Greineder C F. Bivalent engagement of endothelial surface antigens is critical to prolonged surface targeting and protein delivery in vivo. Faseb j. 2020; 34 (9): 11577-93. Epub 2020 Aug. 2. doi: 10.1096/fj.201902515R R. PubMed PMID: 32738178; PMCID: PMC8444999.
B46. Greineder C F, Villa C H, Walsh L R, Kiseleva R Y, Hood E D, Khoshnejad M, Warden-Rothman R, Tsourkas A, Muzykantov V R. Site-Specific Modification of Single-Chain Antibody Fragments for Bioconjugation and Vascular Immunotargeting. Bioconjug Chem. 2018; 29 (1): 56-66. Epub 2017 Dec. 5. doi: 10.1021/acs.bioconjchem.7b00592. PubMed PMID: 29200285; PMCID: PMC6477694.
B47. Greineder C F, Hood E D, Yao A, Khoshnejad M, Brenner J S, Johnston I H, Poncz M, Gottstein C, Muzykantov V R. Molecular engineering of high affinity single-chain antibody fragment for endothelial targeting of proteins and nanocarriers in rodents and humans. J Control Release. 2016; 226:229-37. Epub 2016 Feb. 9. doi: 10.1016/j.jconrel.2016.02.006. PubMed PMID: 26855052; PMCID: PMC4964279.
B48. Pan D, Vargas-Morales O, Zern B, Anselmo A C, Gupta V, Zakrewsky M, Mitragotri S, Muzykantov V. The Effect of Polymeric Nanoparticles on Biocompatibility of Carrier Red Blood Cells. PLOS One. 2016; 11 (3): e0152074. Epub 2016 Mar. 24. doi: 10.1371/journal.pone.0152074. PubMed PMID: 27003833; PMCID: PMC4803339.
B49. Hill B C, Hanna C A, Adamski J, Pham H P, Marques M B, Williams L A, 3rd. Ficin-Treated Red Cells Help Identify Clinically Significant Alloantibodies Masked as Reactions of Undetermined Specificity in Gel Microtubes. Lab Med. 2017; 48 (1): 24-8. Epub 2016 Dec. 23. doi: 10.1093/labmed/lmw062. PubMed PMID: 28007780.
B50. Tong T N, Branch D R. Use of a Monocyte Monolayer Assay to Evaluate Fc γ Receptor-mediated Phagocytosis. J Vis Exp. 2017 (119). Epub 2017 Jan. 25. doi: 10.3791/55039. PubMed PMID: 28117787; PMCID: PMC5408648.
B51. Samdal H H, Michaelsen T E, Heier H E, Nordhagen R. Antibody dependent cell mediated cytotoxicity against anti-D sensitized human erythrocytes. Influence of various effector cells and enzyme treatment of the target cells. Apmis. 1988; 96 (3): 250-6. Epub 1988 Mar. 1. PubMed PMID: 3126786.

SEQUENCES

SEQ ID NO: 1-IdeS
MRKRCYSTSAVVLAAVTLFALSVDRGVIADSFSANQEIRYSEVTPYHVTSVWTKGVTPP
AKFTQGEDVFHAPYVANQGWYDITKTFNGKDDLLCGAATAGNMLHWWFDQNKEKIE
AYLKKHPDKQKIMFGDQELLDVRKVINTKGDQTNSELFNYFRDKAFPGLSARRIGVMP
DLVLDMFINGYYLNVYKTQTTDVNRTYQEKDRRGGIFDAVFTRGDQSKLLTSRHDFKE
KNLKEISDLIKKELTEGKALGLSHTYANVRINHVINLWGADFDSNGNLKAIYVTDSDSN
ASIGMKKYFVGVNSAGKVAISAKEIKEDNIGAQVLGLFTLSTGQDSWNQTN
SEQ ID NO: 2-YTH Fab V_{H}-C_{H1} (amino acid sequence)
EVQLVETGGGLVQPGKSLKLTCATSGFTFSNAWMHWVRQSPEKQLEWVSQIRAKSNN
YATYYAESVKGRFTISRDDSKRNVYLQMNSLKEEDTAIYYCTTGSGDYWGQGVMVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC
SEQ ID NO: 3-YTH Fab V_{H}-C_{H1} (nucleic acid sequence)
gaagtacaactggtagaaacaggaggggggctggtccagcctggcaaaagcctgaaactgacgtgcgctacgagcggctttacattttca
aacgcttggatgcactgggttaggcagtcccctgagaagcaattggaatgggtaagccagatacgagctaagtcaaacaactacgcgacg
tattatgcagagtctgtaaaaggaaggttcacgataagtcgggatgactctaaaaggaacgtgtacctgcaaatgaacagcctcaaggaag
aagatacagccatttactattgtaccacgggctctggagattactggggtcaaggagtcatggtcactgtttcaagtgctagcaccaagggcc
catcggtcttccccctggcaccctcctccaagagcacctctgggggcacagcggccctgggctgcctggtcaaggactacttccccgaac
cggtgacggtgtcgtggaactcaggcgccctgaccagcggcgtgcacaccttcccggctgtcctacagtcctcaggactctactccctcag
cagcgtggtgaccgtgccctccagcagcttgggcacccagacctacatctgcaacgtgaatcacaagcccagcaacaccaaggtggaca
agaaagttgagcccaaatcttgt
SEQ ID NO: 4-YTH Fab V_{H} CDR1
NAWMH
SEQ ID NO: 5-YTH Fab V_{H} CDR2
QIRAKSNNYATYYAESVKG
SEQ ID NO: 6-YTH Fab V_{H} CDR3
GSGDY
SEQ ID NO: 7-YTH Fab V_{L}-C_{L} (amino acid sequence)
DVVLTQTPPTLLATIGQSVSISCRSSQSLLHRSGNTYLNWLLQRTGQSPQPLIYLVSKLES
GVPNRFSGSGSGTDFTLKISGVEAEDLGIYYCMQFTHYPYTFGAGTKLELKRARTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
SEQ ID NO: 8-YTH Fab V_{L}-C_{L} (nucleic acid sequence)
gacgttgttctgactcaaacaccaccaacactccttgcaaccatcggtcaatctgtttcaatatcatgcagaagttcccagtccttgttgcac
cgaagtggtaacacgtacttgaactggctcctccaacggacaggacaaagccctcagccgctcatttatttggtttccaagctggagtccggt
gtaccgaacagattctcaggttccggtagtggcacagacttcaccttgaaaattagtggcgtggaggcggaagacctgggaatttattattgt
atgcaattcactcattacccgtacacgttcggcgcaggcaccaagttggagttgaaacgggcacgtacggtggctgcaccatctgtcttcatc
ttcccgccatctgatgagcagttgaaatctggaactgcctctgttgtgtgcctgctgaataacttctatcccagagaggccaaagtacagtgg
aaggtggataacgccctccaatcgggtaactcccaggagagtgtcacagagcaggacagcaaggacagcacctacagcctcagcagcacc
ctgacgctgagcaaagcagactacgagaaacacaaagtctacgcctgcgaagtcacccatcagggcctgagctcgcccgtcacaaagag
cttcaacaggggagagtgt
SEQ ID NO: 9-YTH Fab V_{L} CDR1
RSSQSLLHRSGNTYLN
SEQ ID NO: 10-YTH Fab V_{L} CDR2
LVSKLES
SEQ ID NO: 11-YTH Fab V_{L} CDR3
MQFTHYPYT
SEQ ID NO: 12-YTH Fab V_{H}-C_{H1}-IdeS fusion (amino acid)
EVQLVETGGGLVQPGKSLKLTCATSGFTFSNAWMHWVRQSPEKQLEWVSQIRAKSNN
YATYYAESVKGRFTISRDDSKRNVYLQMNSLKEEDTAIYYCTTGSGDYWGQGVMVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCAAAGGSDSFSANQEI
RYSEVTPYHVTSVWTKGVTPPANFTQGEDVFHAPYVANQGWYDITKTFNGKDDLLCG
AATAGNMLHWWFDQNKDQIKRYLEEHPEKQKINFNGEQMFDVKEAIDTKNHQLDSKL
FEYFKEKAFPYLSTKHLGVFPDHVIDMFINGYRLSLTNHGPTPVKEGSKDPRGGIFDAVF
TRGDQSKLLTSRHDFKEKNLKEISDLIKKELTEGKALGLSHTYANVRINHVINLWGADF
DSNGNLKAIYVTDSDSNASIGMKKYFVGVNSAGKVAISAKEIKEDNIGAQVLGLFTLST
GQDSWNQTNHHHHHH
SEQ ID NO: 13-YTH Fab V_{H}-C_{H1}-IdeS fusion (nucleic acid)
gaagtacaactggtagaaacaggaggggggctggtccagcctggcaaaagcctgaaactgacgtgcgctacgagcggctttacattttca
aacgcttggatgcactgggttaggcagtcccctgagaagcaattggaatgggtaagccagatacgagctaagtcaaacaactacgcgacg
tattatgcagagtctgtaaaaggaaggttcacgataagtcgggatgactctaaaaggaacgtgtacctgcaaatgaacagcctcaaggaag
aagatacagccatttactattgtaccacgggctctggagattactggggtcaaggagtcatggtcactgtttcaagtgctagcaccaagggcc
catcggtcttccccctggcaccctcctccaagagcacctctgggggcacagcggccctgggctgcctggtcaaggactacttccccgaac
cggtgacggtgtcgtggaactcaggcgccctgaccagcggcgtgcacaccttcccggctgtcctacagtcctcaggactctactccctcag
cagcgtggtgaccgtgccctccagcagcttgggcacccagacctacatctgcaacgtgaatcacaagcccagcaacaccaaggtggaca
agaaagttgagcccaaatcttgtgcggccgctggaggttcagacagcttttccgccaaccaagaaataaggtattccgaggtgacgccttat
catgttacgagtgtgtggaccaagggcgttacacccccggcgaactttacgcagggagaggacgtgttccacgcgccctatgtcgctaatc
aaggttggtacgacataactaagacgtttaatggtaaagatgacttgctttgtggcgcagccacggcaggaaacatgctgcattggtggttcg
atcaaaacaaggatcaaattaagcgctaccttgaggagcatcctgagaaacaaaagatcaacttcaatggggaacagatgttcgatgttaaa
gaggcgatagataccaaaaatcatcaactcgactccaaactcttcgagtattttaaggagaaggcgttcccgtatctgtcaacaaagcacttg
ggggtcttcccggatcatgttattgatatgtttatcaatggatataggctctccctgacaaaccatggccccaccccagtaaaggaaggctcc
aaagatccacggggcggcatttttgatgccgtttttacgcgaggggaccaatcaaaactcctgaccagccggcatgacttcaaagaaaaaaa
cttgaaagagataagtgacttgataaagaaagagctcaccgaagggaaggctttgggactctctcacacttacgcaaacgtgcgcatcaatc
atgtaatcaatctgtggggggccgatttcgatagcaatggaaatcttaaggcgatctatgtaacggattccgattctaatgcgagcattggta
tgaaaaagtatttcgtgggagttaactcagctggtaaggtcgctatttccgcaaaagagatcaaggaggacaacattggtgcgcaagttttgg
gtctctttacgctcagcacaggccaagattcctggaatcagacgaatcatcatcaccatcaccat
SEQ ID NO: 14-Wrb Fab V_{H}-C_{H1} (amino acid)
EVQLLESGPGLVKPSETLSLTCTVSGSSLSSAYGWNWIRQPPGKGLEWIGSIGGSRDNTN
YNPSLKRRVTISKDTSKNQFSLKLKSVTAADTAVYYCAQRGAYGYSYFDYWGQGVLV
AVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC
SEQ ID NO: 15-Wrb Fab V_{H}-C_{H1} (nucleic acid)
gaggtacaactcctggaatctggaccagggcttgtaaagccatccgaaaccctgtcacttacttgtactgtctctggctcatctttgtcaagc
gcctacggttggaattggatacgacaacccccaggaaagggccttgaatggatagggagcatcggaggttcccgcgacaacacgaattaca
atccgagtttgaaacgccgggttacaatatccaaggatacgagcaagaaccagttttctctcaaactgaaatcagtaacagcagctgacact
gccgtatattactgtgctcaacgcggcgcctatggatatagttacttcgactactggggccagggcgtcttggtggcagtctcatcagctagc
accaagggcccatcggtcttccccctggcaccctcctccaagagcacctctgggggcacagcggccctgggctgcctggtcaaggacta
cttccccgaaccggtgacggtgtcgtggaactcaggcgccctgaccagcggcgtgcacaccttcccggctgtcctacagtcctcaggactc
tactccctcagcagcgtggtgaccgtgccctccagcagcttgggcacccagacctacatctgcaacgtgaatcacaagcccagcaacacc
aaggtggacaagaaagttgagcccaaatcttgt
SEQ ID NO: 16-Wrb Fab V_{H} CDR1
SAYGWN
SEQ ID NO: 17-Wrb Fab V_{H} CDR2
SIGGSRDNTNYNPSLKR
SEQ ID NO: 18-Wrb Fab V_{H} CDR3
RGAYGYSYFDY
SEQ ID NO: 19-Wrb Fab V_{L}-C_{L} (amino acid)
ELTLTQSPATLSLSPGETATLSCRASQTVGRNLAWYQQRPGQAPNLLVHSAYFRATGIP
DRFSGSGSGTDFTLTISSLEPEDAGVYHCQQYNDLLPLTFGGGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 20-Wrb Fab V_{L}-C_{L} (nucleic acid)
gagttgacactgacgcagtcaccggcgactcttagtctctcccctggcgaaaccgcgactctttcttgtcgcgcatcccaaacggttggacg
aaatttggcgtggtatcaacagcgccccggacaggccccgaatctgttggtgcattccgcatatttcagggcgactggtattcctgaccgctt
ttccggatcaggttccggaacggatttcacactcactattagttccttggaaccagaagatgcgggagtttaccattgccaacaatataacga
tctgctcccacttactttcggtgggggcaccaaggtagagatcaagcgtacggtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaagc
agactacgagaaacacaaagtctacgcctgcgaagtcacccatcagggcctgagctcgcccgtcacaaagagcttcaacaggggagagt
gt
SEQ ID NO: 21-Wrb Fab V_{L} CDR1
RASQTVGRNLA
SEQ ID NO: 22-Wrb Fab V_{L} CDR2
SAYFRAT
SEQ ID NO: 23-Wrb Fab V_{L} CDR3
QQYNDLLPLT
SEQ ID NO: 24-Wrb Fab V_{H}-C_{H1}-IdeS fusion (amino acid)
EVQLLESGPGLVKPSETLSLTCTVSGSSLSSAYGWNWIRQPPGKGLEWIGSIGGSRDNTN
YNPSLKRRVTISKDTSKNQFSLKLKSVTAADTAVYYCAQRGAYGYSYFDYWGQGVLV
AVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCAAAGGSDSFSAN
QEIRYSEVTPYHVTSVWTKGVTPPANFTQGEDVFHAPYVANQGWYDITKTFNGKDDLL
CGAATAGNMLHWWFDQNKDQIKRYLEEHPEKQKINFNGEQMFDVKEAIDTKNHOLDS
KLFEYFKEKAFPYLSTKHLGVFPDHVIDMFINGYRLSLTNHGPTPVKEGSKDPRGGIFDA
VFTRGDQSKLLTSRHDFKEKNLKEISDLIKKELTEGKALGLSHTYANVRINHVINLWGA
DFDSNGNLKAIYVTDSDSNASIGMKKYFVGVNSAGKVAISAKEIKEDNIGAQVLGLFTL
STGQDSWNQTNHHHHHH
SEQ ID NO: 25-Wrb V_{H}-C_{H1}-IdeS fusion (nucleic acid)
gaggtacaactcctggaatctggaccagggcttgtaaagccatccgaaaccctgtcacttacttgtactgtctctggctcatctttgtcaagc
gcctacggttggaattggatacgacaacccccaggaaagggccttgaatggatagggagcatcggaggttcccgcgacaacacgaattaca
atccgagtttgaaacgccgggttacaatatccaaggatacgagcaagaaccagttttctctcaaactgaaatcagtaacagcagctgacact
gccgtatattactgtgctcaacgcggcgcctatggatatagttacttcgactactggggccagggcgtcttggtggcagtctcatcagctagc
accaagggcccatcggtcttccccctggcaccctcctccaagagcacctctgggggcacagcggccctgggctgcctggtcaaggacta
cttccccgaaccggtgacggtgtcgtggaactcaggcgccctgaccagcggcgtgcacaccttcccggctgtcctacagtcctcaggactc
tactccctcagcagcgtggtgaccgtgccctccagcagcttgggcacccagacctacatctgcaacgtgaatcacaagcccagcaacacc
aaggtggacaagaaagttgagcccaaatcttgtgcggccgctggaggttcagacagcttttccgccaaccaagaaataaggtattccgagg
tgacgccttatcatgttacgagtgtgtggaccaagggcgttacacccccggcgaactttacgcagggagaggacgtgttccacgcgcccta
tgtcgctaatcaaggttggtacgacataactaagacgtttaatggtaaagatgacttgctttgtggcgcagccacggcaggaaacatgctgca
ttggtggttcgatcaaaacaaggatcaaattaagcgctaccttgaggagcatcctgagaaacaaaagatcaacttcaatggggaacagatgt
tcgatgttaaagaggcgatagataccaaaaatcatcaactcgactccaaactcttcgagtattttaaggagaaggcgttcccgtatctgtcaa
caaagcacttgggggtcttcccggatcatgttattgatatgtttatcaatggatataggctctccctgacaaaccatggccccaccccagtaa
aggaaggctccaaagatccacggggcggcatttttgatgccgtttttacgcgaggggaccaatcaaaactcctgaccagccggcatgacttca
aagaaaaaaacttgaaagagataagtgacttgataaagaaagagctcaccgaagggaaggctttgggactctctcacacttacgcaaacgt
gcgcatcaatcatgtaatcaatctgtggggggccgatttcgatagcaatggaaatcttaaggcgatctatgtaacggattccgattctaatgc
gagcattggtatgaaaaagtatttcgtgggagttaactcagctggtaaggtcgctatttccgcaaaagagatcaaggaggacaacattggtgc
gcaagttttgggtctctttacgctcagcacaggccaagattcctggaatcagacgaatcatcatcaccatcaccat
SEQ ID NO: 26-Rh17 Fab V_{H}-C_{H1} (amino acid)
EVQLLESGPGLLKPSETLSLTCAVSGAPISNYWWSWIRQSPGKGLEWIGEIDGSIYTTYY
NPSLKSRVAISKDTSKNRLSLKLTSVTAADTAVYYCAREGQNPLVPTYGSTGFGLDFWG
HGLAVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSC
SEQ ID NO: 27-Rh17 Fab V_{H}-C_{H1} (nucleic acid)
Gaggtacaactcctggaatctggaccagggcttgtaaagccatccgaaaccctgtcacttacttgtactgtctctggctcatctttgtcaagc
gcctacggttggaattggatacgacaacccccaggaaagggccttgaatggatagggagcatcggaggttcccgcgacaacacgaattaca
atccgagtttgaaacgccgggttacaatatccaaggatacgagcaagaaccagttttctctcaaactgaaatcagtaacagcagctgacact
gccgtatattactgtgctcaacgcggcgcctatggatatagttacttcgactactggggccagggcgtcttggtggcagtctcatcagctagc
accaagggcccatcggtcttccccctggcaccctcctccaagagcacctctgggggcacagcggccctgggctgcctggtcaaggacta
cttccccgaaccggtgacggtgtcgtggaactcaggcgccctgaccagcggcgtgcacaccttcccggctgtcctacagtcctcaggactc
tactccctcagcagcgtggtgaccgtgccctccagcagcttgggcacccagacctacatctgcaacgtgaatcacaagcccagcaacacc
aaggtggacaagaaagttgagcccaaatcttgt
SEQ ID NO: 28-Rh17 Fab V_{H} CDR1
NYWWS
SEQ ID NO: 29-Rh17 Fab V_{H} CDR2
EIDGSIYTTYYNPSLKS
SEQ ID NO: 30-Rh17 Fab V_{H} CDR3
EGQNPLVPTYGSTGFGLDF
SEQ ID NO: 31-Rh17 Fab V_{L}-C_{L} (amino acid)
AELTQSPSSLSASVGDRVTITCQASQGISSWLAWYQQKPGKAPKLLIYKASSLQSGVPSR
FSGSGSGTDFTLTISSLQSEDFATYYCQQYSSSPRTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 32-Rh17 Fab V_{L}-C_{L} (nucleic acid)
Gctgagttgacgcagagtcctagtagcctctctgcgtctgtaggagatcgggtcaccatcacatgccaagcatcacaaggtatctcctcatg
gcttgcttggtaccaacagaaaccaggtaaagcacctaagctgcttatatacaaagcctctagcttgcagagtggcgtcccgtcccgctttag
cggctccggctcagggacggactttacattgacgatctcttcacttcagtctgaagattttgcaacctactattgccagcaatactcctcctc
tccgagaactttcggtcagggaacaaaggttgaaataaaacgtacggtggctgcaccatctgtcttcatcttcccgccatctgatgagcagtt
gaaatctggaactgcctctgttgtgtgcctgctgaataacttctatcccagagaggccaaagtacagtggaaggtggataacgccctccaatc
gggtaactcccaggagagtgtcacagagcaggacagcaaggacagcacctacagcctcagcagcaccctgacgctgagcaaagcagacta
cgagaaacacaaagtctacgcctgcgaagtcacccatcagggcctgagctcgcccgtcacaaagagcttcaacaggggagagtgt
SEQ ID NO: 33-Rh17 Fab V_{L} CDR1
QASQGISSWLA
SEQ ID NO: 34-Rh17 Fab V_{L} CDR2
KASSLOS
SEQ ID NO: 35-Rh17 Fab V_{L} CDR3
QQYSSSPRT
SEQ ID NO: 36-Rh17 Fab V_{H}-C_{H1}-IdeS fusion (amino acid)
EVQLLESGPGLLKPSETLSLTCAVSGAPISNYWWSWIRQSPGKGLEWIGEIDGSIYTTYY
NPSLKSRVAISKDTSKNRLSLKLTSVTAADTAVYYCAREGQNPLVPTYGSTGFGLDFWG
HGLAVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCAAAGGS
DSFSANQEIRYSEVTPYHVTSVWTKGVTPPANFTQGEDVFHAPYVANQGWYDITKTEN
GKDDLLCGAATAGNMLHWWFDQNKDQIKRYLEEHPEKQKINFNGEQMEDVKEAIDTK
NHQLDSKLFEYFKEKAFPYLSTKHLGVFPDHVIDMFINGYRLSLTNHGPTPVKEGSKDPR
GGIFDAVFTRGDQSKLLTSRHDFKEKNLKEISDLIKKELTEGKALGLSHTYANVRINHVI
NLWGADFDSNGNLKAIYVTDSDSNASIGMKKYFVGVNSAGKVAISAKEIKEDNIGAQV
LGLFTLSTGQDSWNQTNHHHHHH
SEQ ID NO: 37-Rh17 V_{H}-C_{H1}-IdeS fusion (nucleic acid)
gaagtgcaattgctggagagcgggccgggccttctcaaaccaagtgaaactctgtcccttacctgcgcggtctcaggggcgcccataagc
aattattggtggtcctggatacgccagagtcctggtaaaggcttggagtggattggggaaattgatggctctatctatacaacatattacaac
ccgtcattgaagagccgcgtggctatttcaaaagacacatccaagaaccgactttctctgaagctcacatctgtaactgccgccgatacggca
gtatattactgcgccagggaaggacagaatcctcttgtaccaacttatggcagcacaggattcggactcgacttttggggccatggattggcg
gttacggtgagttctgctagcaccaagggcccatcggtcttccccctggcaccctcctccaagagcacctctgggggcacagcggccctg
ggctgcctggtcaaggactacttccccgaaccggtgacggtgtcgtggaactcaggcgccctgaccagcggcgtgcacaccttcccggct
gtcctacagtcctcaggactctactccctcagcagcgtggtgaccgtgccctccagcagcttgggcacccagacctacatctgcaacgtga
atcacaagcccagcaacaccaaggtggacaagaaagttgagcccaaatcttgtgcggccgctggaggttcagacagcttttccgccaacc
aagaaataaggtattccgaggtgacgccttatcatgttacgagtgtgtggaccaagggcgttacacccccggcgaactttacgcagggaga
ggacgtgttccacgcgccctatgtcgctaatcaaggttggtacgacataactaagacgtttaatggtaaagatgacttgctttgtggcgcagc
cacggcaggaaacatgctgcattggtggttcgatcaaaacaaggatcaaattaagcgctaccttgaggagcatcctgagaaacaaaagatca
acttcaatggggaacagatgttcgatgttaaagaggcgatagataccaaaaatcatcaactcgactccaaactcttcgagtattttaaggaga
aggcgttcccgtatctgtcaacaaagcacttgggggtcttcccggatcatgttattgatatgtttatcaatggatataggctctccctgacaa
accatggccccaccccagtaaaggaaggctccaaagatccacggggggcatttttgatgccgtttttacgcgaggggaccaatcaaaactcct
gaccagccggcatgacttcaaagaaaaaaacttgaaagagataagtgacttgataaagaaagagctcaccgaagggaaggctttgggactc
tctcacacttacgcaaacgtgcgcatcaatcatgtaatcaatctgtggggggccgatttcgatagcaatggaaatcttaaggcgatctatgta
acggattccgattctaatgcgagcattggtatgaaaaagtatttcgtgggagttaactcagctggtaaggtcgctatttccgcaaaagagatc
aaggaggacaacattggtgcgcaagttttgggtctctttacgctcagcacaggccaagattcctggaatcagacgaatcatcatcaccatcac
cat
SEQ ID NO: 38-Ter119 Fab V_{H}-C_{H1} (amino acid)
QVKLQESGGGLVQPGGSLKLSCVASGFTFRDHWMNWVRQAPGKTMEWIGDIRPDGSD
TNYAPSVRNRFTISRDNARSILYLQMSNMRSDYTATYYCVRDSPTRAGLMDAWGQGAS
VTVLTASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSC
SEQ ID NO: 39-Ter119 Fab V_{H}-C_{H1} (nucleic acid)
Caagtaaaattgcaggagagtggtgggggactcgtacagccagggggttcccttaaattgtcatgcgttgcctctggttttaccttcagagat
cattggatgaactgggtcagacaagcgcctggtaaaactatggaatggattggggacatacgcccagatggatctgacactaactatgcac
caagtgttcgcaatagatttacaatatctagagataatgcccgatctatcttgtatttgcagatgagcaacatgcgaagtgactatacggcaa
cgtactactgtgtaagagattcaccaactcgagcgggacttatggacgcatggggccagggtgcaagtgtgaccgtactcacggctagcacca
agggcccatcggtcttccccctggcaccctcctccaagagcacctctgggggcacagcggccctgggctgcctggtcaaggactacttcc
ccgaaccggtgacggtgtcgtggaactcaggcgccctgaccagcggcgtgcacaccttcccggctgtcctacagtcctcaggactctactc
cctcagcagcgtggtgaccgtgccctccagcagcttgggcacccagacctacatctgcaacgtgaatcacaagcccagcaacaccaaggt
ggacaagaaagttgagcccaaatcttgt
SEQ ID NO: 40-Ter119 Fab V_{H} CDR1
DHWMN
SEQ ID NO: 41-Ter119 Fab V_{H} CDR2
DIRPDGSDTNYAPSVRN
SEQ ID NO: 42-Ter119 Fab V_{H} CDR3
DSPTRAGLMDA
SEQ ID NO: 43-Ter119 Fab V_{L}-C_{L} (amino acid)
DIQMIQSPSVLSASVGDRVTLNCKASQNINKYLNWYQQKLGEAPKVLIYNTNNLQTGIP
SRFSGSGSGTDFTLTISSLQPEDFATYFCFQHYTWPTFGGGTKLELKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 44-Ter119 Fab V_{L}-C_{L} (nucleic acid)
Gacatacaaatgatacagtctccatctgtcctctctgcgagcgtcggagatcgcgtcaccttgaactgtaaggcgtctcagaatattaacaag
tatttgaactggtatcagcagaagctcggtgaggcccctaaggtactcatatacaacactaataaccttcagacaggcattccaagccgattt
agcggttccggaagtggaactgacttcacactgaccatctccagcctccaacccgaggattttgctacgtatttctgtttccagcattatact
tggcctaccttcggcggcggtacgaaactggaactgaagcgtacggtggctgcaccatctgtcttcatcttcccgccatctgatgagcagttg
aaatctggaactgcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaagcagactac
gagaaacacaaagtctacgcctgcgaagtcacccatcagggcctgagctcgcccgtcacaaagagcttcaacaggggagagtgt
SEQ ID NO: 45-Ter119 Fab V_{L} CDR1
DHWMN
SEQ ID NO: 46-Ter119 Fab V_{L} CDR2
DIRPDGSDTNYAPSVRN
SEQ ID NO: 47-Ter119 Fab V_{L} CDR3
DSPTRAGLMDA
SEQ ID NO: 48-Ter119 Fab V_{H}-C_{H1}-IdeS fusion (amino acid)
QVKLQESGGGLVQPGGSLKLSCVASGFTFRDHWMNWVRQAPGKTMEWIGDIRPDGSD
TNYAPSVRNRFTISRDNARSILYLQMSNMRSDYTATYYCVRDSPTRAGLMDAWGQGAS
VTVLTASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCAAAGGSDSFSA
NQEIRYSEVTPYHVTSVWTKGVTPPANFTQGEDVFHAPYVANQGWYDITKTFNGKDDL
LCGAATAGNMLHWWFDQNKDQIKRYLEEHPEKQKINFNGEQMFDVKEAIDTKNHQLD
SKLFEYFKEKAFPYLSTKHLGVFPDHVIDMFINGYRLSLTNHGPTPVKEGSKDPRGGIFD
AVFTRGDQSKLLTSRHDFKEKNLKEISDLIKKELTEGKALGLSHTYANVRINHVINLWG
ADFDSNGNLKAIYVTDSDSNASIGMKKYFVGVNSAGKVAISAKEIKEDNIGAQVLGLFT
LSTGQDSWNQTNHHHHHH
SEQ ID NO: 49-Ter119 V_{H}-C_{H1}-IdeS fusion (nucleic acid)
caagtaaaattgcaggagagtggtgggggactcgtacagccagggggttcccttaaattgtcatgcgttgcctctggttttaccttcagagat
cattggatgaactgggtcagacaagcgcctggtaaaactatggaatggattggggacatacgcccagatggatctgacactaactatgcacc
aagtgttcgcaatagatttacaatatctagagataatgcccgatctatcttgtatttgcagatgagcaacatgcgaagtgactatacggcaac
gtactactgtgtaagagattcaccaactcgagcgggacttatggacgcatggggccagggtgcaagtgtgaccgtactcacggctagcacca
agggcccatcggtcttccccctggcaccctcctccaagagcacctctgggggcacagcggccctgggctgcctggtcaaggactacttcc
ccgaaccggtgacggtgtcgtggaactcaggcgccctgaccagcggcgtgcacaccttcccggctgtcctacagtcctcaggactctactc
cctcagcagcgtggtgaccgtgccctccagcagcttgggcacccagacctacatctgcaacgtgaatcacaagcccagcaacaccaaggt
ggacaagaaagttgagcccaaatcttgtgcggccgctggaggttcagacagcttttccgccaaccaagaaataaggtattccgaggtgacg
ccttatcatgttacgagtgtgtggaccaagggcgttacacccccggcgaactttacgcagggagaggacgtgttccacgcgccctatgtcg
ctaatcaaggttggtacgacataactaagacgtttaatggtaaagatgacttgctttgtggcgcagccacggcaggaaacatgctgcattggt
ggttcgatcaaaacaaggatcaaattaagcgctaccttgaggagcatcctgagaaacaaaagatcaacttcaatggggaacagatgttcgat
gttaaagaggcgatagataccaaaaatcatcaactcgactccaaactcttcgagtattttaaggagaaggcgttcccgtatctgtcaacaaag
cacttgggggtcttcccggatcatgttattgatatgtttatcaatggatataggctctccctgacaaaccatggccccaccccagtaaaggaa
ggctccaaagatccacggggcggcatttttgatgccgtttttacgcgaggggaccaatcaaaactcctgaccagccggcatgacttcaaagaa
aaaaacttgaaagagataagtgacttgataaagaaagagctcaccgaagggaaggctttgggactctctcacacttacgcaaacgtgcgca
tcaatcatgtaatcaatctgtggggggccgatttcgatagcaatggaaatcttaaggcgatctatgtaacggattccgattctaatgcgagca
ttggtatgaaaaagtatttcgtgggagttaactcagctggtaaggtcgctatttccgcaaaagagatcaaggaggacaacattggtgcgcaag
ttttgggtctctttacgctcagcacaggccaagattcctggaatcagacgaatcatcatcaccatcaccat
SEQ ID NO: 50-34-3C Fab V_{H}-C_{H1} (amino acid)
EVQLQQSGPELVKPGASVKISCKVSGYSFTDYNVDWVKQSHRKTLEWIGYIYPDNGVT
DYTQKFNTKATLTVDKSSSTAYMDLHSLTSEDSAVYYCARGRGWYFDVWGAGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC
SEQ ID NO: 51-34-3C Fab V_{H}-C_{H1} (nucleic acid)
gaggtgcagctccagcagtcaggtcccgagctggtaaagcccggagcctcagtgaagatctcttgtaaggtatctggatacagtttcacgg
attacaacgttgattgggttaagcagtctcacagaaagaccctggaatggataggctatatttatccagataatggtgttacagactatacac
aaaaattcaacactaaggcgacactcacagtcgataaaagttcttccaccgcgtatatggacttgcactctctgacttcagaagattccgccg
tctactactgtgccagagggaggggctggtactttgacgtgtggggcgcgggcactactgtcacagtgagcagcgctagcaccaagggccca
tcggtcttccccctggcaccctcctccaagagcacctctgggggcacagcggccctgggctgcctggtcaaggactacttccccgaaccg
gtgacggtgtcgtggaactcaggcgccctgaccagcggcgtgcacaccttcccggctgtcctacagtcctcaggactctactccctcagca
gcgtggtgaccgtgccctccagcagcttgggcacccagacctacatctgcaacgtgaatcacaagcccagcaacaccaaggtggacaag
aaagttgagcccaaatcttgt
SEQ ID NO: 52-34-3C Fab V_{H} CDR1
DYNVD
SEQ ID NO: 53-34-3C Fab V_{H} CDR2
YIYPDNGVTDYTQKENT
SEQ ID NO: 54-34-3C Fab V_{H} CDR3
GRGWYFDV
SEQ ID NO: 55-34-3C Fab V_{L}-C_{L} (amino acid)
DIVLTQSPASLAVSLGQRATISCRASESVDNYGISFMNWLQQKPGQPPKLLIYAASNQVS
GVPARFSGSGSGTDFSLNIHPMEEDDTAMYFCQQSKEIPYTFGGGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 56-34-3C Fab V_{L}-C_{L} (nucleic acid)
Gacattgttttgacccagagcccggccagcttggctgtgagtctgggccaacgcgctacaatctcctgtcgcgcgagtgaatcagtggata
attatggtatttcattcatgaattggctgcaacagaaaccgggacaacctccaaagctccttatatatgcggcttccaatcaagtaagcgggg
tcccagcccggttttctggttccggctccggtactgactttagtttgaacattcacccaatggaagaagatgataccgccatgtacttctgcc
aacaatcaaaggaaattccttatacctttggcggcggcacgaagctggagattaagcgtacggtggctgcaccatctgtcttcatcttcccgc
catctgatgagcagttgaaatctggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccctgacgctg
agcaaagcagactacgagaaacacaaagtctacgcctgcgaagtcacccatcagggcctgagctcgcccgtcacaaagagcttcaacag
gggagagtgt
SEQ ID NO: 57-34-3C Fab V_{L} CDR1
RASESVDNYGISFMN
SEQ ID NO: 58-34-3C Fab V_{L} CDR2
AASNQVS
SEQ ID NO: 59-34-3C Fab V_{L} CDR3
QQSKEIPYT
SEQ ID NO: 60-34-3C Fab V_{H}-C_{H1}-IdeS fusion (amino acid)
EVQLQQSGPELVKPGASVKISCKVSGYSFTDYNVDWVKQSHRKTLEWIGYIYPDNGVT
DYTQKFNTKATLTVDKSSSTAYMDLHSLTSEDSAVYYCARGRGWYFDVWGAGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCAAAGGSDSFSANQEI
RYSEVTPYHVTSVWTKGVTPPANFTQGEDVFHAPYVANQGWYDITKTFNGKDDLLCG
AATAGNMLHWWFDQNKDQIKRYLEEHPEKQKINFNGEQMFDVKEAIDTKNHQLDSKL
FEYFKEKAFPYLSTKHLGVFPDHVIDMFINGYRLSLTNHGPTPVKEGSKDPRGGIFDAVF
TRGDQSKLLTSRHDFKEKNLKEISDLIKKELTEGKALGLSHTYANVRINHVINLWGADF
DSNGNLKAIYVTDSDSNASIGMKKYFVGVNSAGKVAISAKEIKEDNIGAQVLGLFTLST
GQDSWNQTNHHHHHH
SEQ ID NO: 61-34-3C V_{H}-C_{H1}-IdeS fusion (nucleic acid)
gaggtgcagctccagcagtcaggtcccgagctggtaaagcccggagcctcagtgaagatctcttgtaaggtatctggatacagtttcacgg
attacaacgttgattgggttaagcagtctcacagaaagaccctggaatggataggctatatttatccagataatggtgttacagactatacac
aaaaattcaacactaaggcgacactcacagtcgataaaagttcttccaccgcgtatatggacttgcactctctgacttcagaagattccgccg
tctactactgtgccagagggaggggctggtactttgacgtgtggggcgcgggcactactgtcacagtgagcagcgctagcaccaagggccca
tcggtcttccccctggcaccctcctccaagagcacctctgggggcacagcggccctgggctgcctggtcaaggactacttccccgaaccg
gtgacggtgtcgtggaactcaggcgccctgaccagcggcgtgcacaccttcccggctgtcctacagtcctcaggactctactccctcagca
gcgtggtgaccgtgccctccagcagcttgggcacccagacctacatctgcaacgtgaatcacaagcccagcaacaccaaggtggacaag
aaagttgagcccaaatcttgtgcggccgctggaggttcagacagcttttccgccaaccaagaaataaggtattccgaggtgacgccttatcat
gttacgagtgtgtggaccaagggcgttacacccccggcgaactttacgcagggagaggacgtgttccacgcgccctatgtcgctaatcaag
gttggtacgacataactaagacgtttaatggtaaagatgacttgctttgtggcgcagccacggcaggaaacatgctgcattggtggttcgatc
aaaacaaggatcaaattaagcgctaccttgaggagcatcctgagaaacaaaagatcaacttcaatggggaacagatgttcgatgttaaaga
ggcgatagataccaaaaatcatcaactcgactccaaactcttcgagtattttaaggagaaggcgttcccgtatctgtcaacaaagcacttggg
ggtcttcccggatcatgttattgatatgtttatcaatggatataggctctccctgacaaaccatggccccaccccagtaaaggaaggctccaa
agatccacggggcggcatttttgatgccgtttttacgcgaggggaccaatcaaaactcctgaccagccggcatgacttcaaagaaaaaaactt
gaaagagataagtgacttgataaagaaagagctcaccgaagggaaggctttgggactctctcacacttacgcaaacgtgcgcatcaatcat
gtaatcaatctgtggggggccgatttcgatagcaatggaaatcttaaggcgatctatgtaacggattccgattctaatgcgagcattggtatg
aaaaagtatttcgtgggagttaactcagctggtaaggtcgctatttccgcaaaagagatcaaggaggacaacattggtgcgcaagttttgggt
ctctttacgctcagcacaggccaagattcctggaatcagacgaatcatcatcaccatcaccat
SEQ ID NO: 62 IV.3 scFv
QVQLVQSGAEVKKPGASVKVSCKASGYTFTNYGMNWVRQAPGQRLEWMGWLNTYT
GESWYPDDFKGRVTITRDTSASTAYMELSSLRSEDTAVYYCARGDYGYDDPLDYWGQ
GTLVTVSSGAGGSGGGGSGGGGSGGGGSDIVMTQSPLSLPVTPGEPASISCRSSKSLLHT
NQNTYLHWFLQKPGQSPQLLIYRMSVLASGVPDRFSGSGSGTDFTLKISRVEAEDVGVY
YCMQHLEYPLTFGQGTKLEIKR
SEQ ID NO: 63-IV.3 scFv (with C-term LPET)
QVQLVQSGAEVKKPGASVKVSCKASGYTFTNYGMNWVRQAPGQRLEWMGWLNTYT
GESWYPDDFKGRVTITRDTSASTAYMELSSLRSEDTAVYYCARGDYGYDDPLDYWGQ
GTLVTVSSGAGGSGGGGSGGGGSGGGGSDIVMTQSPLSLPVTPGEPASISCRSSKSLLHT
NQNTYLHWFLQKPGQSPQLLIYRMSVLASGVPDRFSGSGSGTDFTLKISRVEAEDVGVY
YCMQHLEYPLTFGQGTKLEIKRGSSSGLPETGGGHHHHHH
SEQ ID NO: 64-IV.3 scFv (with C-term GGG)
GGGSSGQVQLVQSGAEVKKPGASVKVSCKASGYTFTNYGMNWVRQAPGQRLEWMG
WLNTYTGESWYPDDFKGRVTITRDTSASTAYMELSSLRSEDTAVYYCARGDYGYDDPL
DYWGQGTLVTVSSGAGGSGGGGSGGGGSGGGGSDIVMTQSPLSLPVTPGEPASISCRSS
KSLLHTNQNTYLHWFLQKPGQSPQLLIYRMSVLASGVPDRFSGSGSGTDFTLKISRVEAE
DVGVYYCMQHLEYPLTFGQGTKLEIKRHHHHHH
SEQ ID NO: 65 IV.3 scFv VH CDR1
NYGMN
SEQ ID NO: 66 IV.3 scFv VH CDR2
WLNTYTGESWYPDDFKG
SEQ ID NO: 67 IV.3 scFv VH CDR3
GDYGYDDPLDY
SEQ ID NO: 68 IV.3 scFv VL CDR1
RSSKSLLHTNQNTYLH
SEQ ID NO: 69 IV.3 scFv VL CDR2
RMSVLAS
SEQ ID NO: 70 IV.3 scFv VL CDR3
MQHLEYPLT
SEQ ID NO: 71-IdeS-LPET
DSFSANQEIRYSEVTPYHVTSVWTKGVTPPANFTQGEDVFHAPYVANQGWYDITKTFN
GKDDLLCGAATAGNMLHWWFDQNKDQIKRYLEEHPEKQKINFNGEQMEDVKEAIDTK
NHQLDSKLFEYFKEKAFPYLSTKHLGVFPDHVIDMFINGYRLSLTNHGPTPVKEGSKDPR
GGIFDAVFTRGDQSKLLTSRHDFKEKNLKEISDLIKKELTEGKALGLSHTYANVRINHVI
NLWGADFDSNGNLKAIYVTDSDSNASIGMKKYFVGVNSAGKVAISAKEIKEDNIGAQV
LGLFTLSTGQDSWNQTNGSSSGLPETGGGHHHHHH
SEQ ID NO: 72-GGG-IdeS
GGGSSDSFSANQEIRYSEVTPYHVTSVWTKGVTPPANFTQGEDVFHAPYVANQGWYDI
TKTFNGKDDLLCGAATAGNMLHWWFDQNKDQIKRYLEEHPEKQKINFNGEQMFDVKE
AIDTKNHOLDSKLFEYFKEKAFPYLSTKHLGVFPDHVIDMFINGYRLSLTNHGPTPVKEG
SKDPRGGIFDAVFTRGDQSKLLTSRHDFKEKNLKEISDLIKKELTEGKALGLSHTYANVR
INHVINLWGADFDSNGNLKAIYVTDSDSNASIGMKKYFVGVNSAGKVAISAKEIKEDNI
GAQVLGLFTLSTGQDSWNQTNHHHHHH
SEQ ID NO: 73-IV.3-IdeS conjugate
QVQLVQSGAEVKKPGASVKVSCKASGYTFTNYGMNWVRQAPGQRLEWMGWLNTYT
GESWYPDDFKGRVTITRDTSASTAYMELSSLRSEDTAVYYCARGDYGYDDPLDYWGQ
GTLVTVSSGAGGSGGGGSGGGGSGGGGSDIVMTQSPLSLPVTPGEPASISCRSSKSLLHT
NQNTYLHWFLQKPGQSPQLLIYRMSVLASGVPDRFSGSGSGTDFTLKISRVEAEDVGVY
YCMQHLEYPLTFGQGTKLEIKRGSSSGLPETGGGSSDSFSANQEIRYSEVTPYHVTSVWT
KGVTPPANFTQGEDVFHAPYVANQGWYDITKTFNGKDDLLCGAATAGNMLHWWFDQ
NKDQIKRYLEEHPEKQKINFNGEQMFDVKEAIDTKNHQLDSKLFEYFKEKAFPYLSTKH
LGVFPDHVIDMFINGYRLSLTNHGPTPVKEGSKDPRGGIFDAVFTRGDQSKLLTSRHDFK
EKNLKEISDLIKKELTEGKALGLSHTYANVRINHVINLWGADFDSNGNLKAIYVTDSDSN
ASIGMKKYFVGVNSAGKVAISAKEIKEDNIGAQVLGLFTLSTGQDSWNQTNHHHHHH
SEQ ID NO: 74-IdeS-IV.3 conjugate
DSFSANQEIRYSEVTPYHVTSVWTKGVTPPANFTQGEDVFHAPYVANQGWYDITKTEN
GKDDLLCGAATAGNMLHWWFDQNKDQIKRYLEEHPEKQKINFNGEQMFDVKEAIDTK
NHQLDSKLFEYFKEKAFPYLSTKHLGVFPDHVIDMFINGYRLSLTNHGPTPVKEGSKDPR
GGIFDAVFTRGDQSKLLTSRHDFKEKNLKEISDLIKKELTEGKALGLSHTYANVRINHVI
NLWGADFDSNGNLKAIYVTDSDSNASIGMKKYFVGVNSAGKVAISAKEIKEDNIGAQV
LGLFTLSTGQDSWNQTNGSSSGLPETGGGSSGQVQLVQSGAEVKKPGASVKVSCKASG
YTFTNYGMNWVRQAPGQRLEWMGWLNTYTGESWYPDDFKGRVTITRDTSASTAYME
LSSLRSEDTAVYYCARGDYGYDDPLDYWGQGTLVTVSSGAGGSGGGGSGGGGSGGGG
SDIVMTQSPLSLPVTPGEPASISCRSSKSLLHTNQNTYLHWFLQKPGQSPQLLIYRMSVLA
SGVPDRFSGSGSGTDFTLKISRVEAEDVGVYYCMQHLEYPLTFGQGTKLEIKRHHHHHH
SEQ ID NO: 75-Ab37 anti-PECAM Fab V_{H}-C_{H1} (amino acid sequence)
QVQLQQPGAVLVKPGASVKMSCTASGYTFTNYNMHWVKQTPGQGLEWIGGIYPGNGN
TAYNQKFKGKATVTADKSSSAVHMQVSSLTSEDSAVYYCARRSGNSYSYWYFDVWGA
GTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSC
SEQ ID NO: 76-Ab37 anti-PECAM Fab V_{H}-C_{H1} (nucleic acid sequence)
caggtgcaactgcagcagcctggggctgtgctggtgaagcctggggcctcagtgaagatgtcctgcacggcttctggctacacatttacca
attacaatatgcactgggtaaaacagacacctggacagggcctggaatggattggaggtatttatccaggaaatggtaatactgcctacaatc
agaaattcaaaggcaaggccacagtgactgcagacaaatcctccagcgcagtccacatgcaggtcagcagcctgacatctgaggactctg
cggtctattactgtgcacgacgctccggtaatagctactcctactggtacttcgatgtctggggcgcagggaccacggtcaccgtctcctcgg
ctagcaccaagggcccatcggtcttccccctggcaccctcctccaagagcacctctgggggcacagcggccctgggctgcctggtcaag
gactacttccccgaaccggtgacggtgtcgtggaactcaggcgccctgaccagcggcgtgcacaccttcccggctgtcctacagtcctcag
gactctactccctcagcagcgtggtgaccgtgccctccagcagcttgggcacccagacctacatctgcaacgtgaatcacaagcccagca
acaccaaggtggacaagaaagttgagcccaaatcttgt
SEQ ID NO: 77-Ab37 anti-PECAM Fab V_{H} CDR1
NYNMH
SEQ ID NO: 78-Ab37 anti-PECAM Fab V_{H} CDR2
GIYPGNGNTAYNQKFKG
SEQ ID NO: 79-Ab37 anti-PECAM Fab V_{H} CDR3
RSGNSYSYWYFDV
SEQ ID NO: 80-Ab37 anti-PECAM Fab V_{L}-C_{L} (amino acid sequence)
DIKMTQSPSSIYASLGERVTITCKASQDINNYLTWYLQKPGKSPKTLIYRANRLVDGVPS
RFSGSGSGQDYSLTISSLEFEDMGIYYCLQYDELYTFGGGTKVELRRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
SEQ ID NO: 81-Ab37 anti-PECAM Fab V_{L}-C_{L} (nucleic acid sequence)
Gatatcaagatgacccagtctccatcttccatatatgcatctcttggagagagagtcactatcacttgcaaggcgagtcaggacattaataac
tatttaacctggtacctgcagaaaccagggaaatctcctaagaccctgatctatcgtgcaaacagattggtagatggggtcccatcaaggttc
agtggcagtggatctggacaagattattctctcaccatcagcagcctggagtttgaagatatgggaatttattactgtctacagtatgatgag
ttgtacacgttcggaggtggaaccaaggtcgaattgaggcgtacggtggctgcaccatctgtcttcatcttcccgccatctgatgagcagttg
aaatctggaactgcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaagcagactac
gagaaacacaaagtctacgcctgcgaagtcacccatcagggcctgagctcgcccgtcacaaagagcttcaacaggggagagtgt
SEQ ID NO: 82-Ab37 anti-PECAM Fab V_{L} CDR1
KASQDINNYLT
SEQ ID NO: 83-Ab37 anti-PECAM Fab V_{L} CDR2
RANRLVD
SEQ ID NO: 84-Ab37 anti-PECAM Fab V_{L} CDR3
LQYDELYT
SEQ ID NO: 85-Ab37 anti-PECAM Fab V_{H}-C_{H1}-IdeS fusion (amino acid)
QVQLQQPGAVLVKPGASVKMSCTASGYTFTNYNMHWVKQTPGQGLEWIGGIYPGNGN
TAYNQKFKGKATVTADKSSSAVHMQVSSLTSEDSAVYYCARRSGNSYSYWYFDVWGA
GTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCAAAGGSDSF
SANQEIRYSEVTPYHVTSVWTKGVTPPANFTQGEDVFHAPYVANQGWYDITKTFNGKD
DLLCGAATAGNMLHWWFDQNKDQIKRYLEEHPEKQKINFNGEQMFDVKEAIDTKNHQ
LDSKLFEYFKEKAFPYLSTKHLGVFPDHVIDMFINGYRLSLTNHGPTPVKEGSKDPRGGI
FDAVFTRGDQSKLLTSRHDFKEKNLKEISDLIKKELTEGKALGLSHTYANVRINHVINLW
GADFDSNGNLKAIYVTDSDSNASIGMKKYFVGVNSAGKVAISAKEIKEDNIGAQVLGLF
TLSTGQDSWNQTNHHHHHH
SEQ ID NO: 86-Ab37 anti-PECAM Fab V_{H}-C_{H1}-IdeS fusion (nucleic acid)
caggtgcaactgcagcagcctggggctgtgctggtgaagcctggggcctcagtgaagatgtcctgcacggcttctggctacacatttacca
attacaatatgcactgggtaaaacagacacctggacagggcctggaatggattggaggtatttatccaggaaatggtaatactgcctacaatc
agaaattcaaaggcaaggccacagtgactgcagacaaatcctccagcgcagtccacatgcaggtcagcagcctgacatctgaggactctg
cggtctattactgtgcacgacgctccggtaatagctactcctactggtacttcgatgtctggggcgcagggaccacggtcaccgtctcctcgg
ctagcaccaagggcccatcggtcttccccctggcaccctcctccaagagcacctctgggggcacagcggccctgggctgcctggtcaag
gactacttccccgaaccggtgacggtgtcgtggaactcaggcgccctgaccagcggcgtgcacaccttcccggctgtcctacagtcctcag
gactctactccctcagcagcgtggtgaccgtgccctccagcagcttgggcacccagacctacatctgcaacgtgaatcacaagcccagca
acaccaaggtggacaagaaagttgagcccaaatcttgtgcggccgctggaggttcagacagcttttccgccaaccaagaaataaggtattc
cgaggtgacgccttatcatgttacgagtgtgtggaccaagggcgttacacccccggcgaactttacgcagggagaggacgtgttccacgc
gccctatgtcgctaatcaaggttggtacgacataactaagacgtttaatggtaaagatgacttgctttgtggcgcagccacggcaggaaacat
gctgcattggtggttcgatcaaaacaaggatcaaattaagcgctaccttgaggagcatcctgagaaacaaaagatcaacttcaatggggaac
agatgttcgatgttaaagaggcgatagataccaaaaatcatcaactcgactccaaactcttcgagtattttaaggagaaggcgttcccgtatc
tgtcaacaaagcacttgggggtcttcccggatcatgttattgatatgtttatcaatggatataggctctccctgacaaaccatggccccaccc
cagtaaaggaaggctccaaagatccacggggcggcatttttgatgccgtttttacgcgaggggaccaatcaaaactcctgaccagccggcatg
acttcaaagaaaaaaacttgaaagagataagtgacttgataaagaaagagctcaccgaagggaaggctttgggactctctcacacttacgca
aacgtgcgcatcaatcatgtaatcaatctgtggggggccgatttcgatagcaatggaaatcttaaggcgatctatgtaacggattccgattct
aatgcgagcattggtatgaaaaagtatttcgtgggagttaactcagctggtaaggtcgctatttccgcaaaagagatcaaggaggacaacatt
ggtgcgcaagttttgggtctctttacgctcagcacaggccaagattcctggaatcagacgaatcatcatcaccatcaccat
SEQ ID NO: 87-Ab62 anti-PECAM Fab V_{H}-C_{H1} (amino acid sequence)
QVQLQQSGAELMKPGASVKISCKATGYTFSHYWIEWVKQRPGHGLEWIGEILPVTGSTK
YTEKFKDKATFTADTFSNTAYMQLTSLTSEDSAVYYCARDPHYSGSTYDAMDYWGQG
TSVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSC
SEQ ID NO: 88-Ab62 anti-PECAM Fab V_{H}-C_{H1} (nucleic acid sequence)
caggtgcagcttcaacaaagcggagcagagctgatgaagccaggcgctagtgtgaagatctcatgcaaggccaccggctacacgttcag
ccactattggatcgagtgggtgaaacagaggcccggtcatggcctggagtggatcggcgagatcctgcccgtgaccggctccaccaagta
caccgagaagttcaaggacaaggctaccttcaccgccgacaccttcagcaataccgcctacatgcaactgacaagcctgaccagcgagg
acagcgccgtgtactactgtgccagggaccctcactacagcggcagcacctacgacgccatggactactggggccagggcaccagcgta
acagtgagcgccgctagcaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggctgt
cctacagtcctcaggactctactccctcagcagcgtggtgaccgtgccctccagcagcttgggcacccagacctacatctgcaacgtgaatc
acaagcccagcaacaccaaggtggacaagaaagttgagcccaaatcttgt
SEQ ID NO: 89-Ab62 anti-PECAM Fab V_{H} CDR1
HYWIE
SEQ ID NO: 90-Ab62 anti-PECAM Fab V_{H} CDR2
EILPVTGSTKYTEKFKD
SEQ ID NO: 91-Ab62 anti-PECAM Fab V_{H} CDR3
DPHYSGSTYDAMDY
SEQ ID NO: 92-Ab62 anti-PECAM Fab V_{L}-C_{L} (amino acid sequence)
NIVLTQSPASLAVSLGQRATISCRASESVDSYGNSFMHWYQQKPGQPPKLLIYLASNLES
GVPARFSGSGSRTDFTLTIDPVEADDAATYYCQQNNEDPWTFGGGTKLEIKRRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 93-Ab62 anti-PECAM Fab V_{L}-C_{L} (nucleic acid sequence)
aacatcgtgctgacccagagccccgcatctctggccgtgagcctgggccaaagagccaccatcagctgtagcgccagcgagagcgtgg
acagctatggcaacaacttcatgcactggtatcagcaaaagcctggccaaccccccaagctgctgatctacctggcaagcaacctggaga
gcggcgtgcccgtgaggttcagcggcagcgggagcaggaccgacttcaccctgaccatcgaccccgtggaggccgacgacgccgcca
cttactactgccaacagaacaacgaggacccctggaccttcggtgggggtacgaagttggagatcaagaggcgtacggtggctgcaccat
ctgtcttcatcttcccgccatctgatgagcagttgaaatctggaactgcctctgttgtgtgcctgctgaataacttctatcccagagaggcc
aaagtacagtggaaggtggataacgccctccaatcgggtaactcccaggagagtgtcacagagcaggacagcaaggacagcacctacagcct
cagcagcaccctgacgctgagcaaagcagactacgagaaacacaaagtctacgcctgcgaagtcacccatcagggcctgagctcgccc
gtcacaaagagcttcaacaggggagagtgt
SEQ ID NO: 94-Ab62 anti-PECAM Fab V_{L} CDR1
RASESVDSYGNSFMH
SEQ ID NO: 95-Ab62 anti-PECAM Fab V_{L} CDR2
LASNLES
SEQ ID NO: 96-Ab62 anti-PECAM Fab V_{L} CDR3
QQNNEDPWT
SEQ ID NO: 97-Ab62 anti-PECAM Fab V_{H}-C_{H1}-IdeS fusion (amino acid)
QVQLQQSGAELMKPGASVKISCKATGYTFSHYWIEWVKQRPGHGLEWIGEILPVTGSTK
YTEKFKDKATFTADTFSNTAYMQLTSLTSEDSAVYYCARDPHYSGSTYDAMDYWGQG
TSVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCAAAGGSDSFS
ANQEIRYSEVTPYHVTSVWTKGVTPPANFTQGEDVFHAPYVANQGWYDITKTFNGKDD
LLCGAATAGNMLHWWFDQNKDQIKRYLEEHPEKQKINFNGEQMFDVKEAIDTKNHQL
DSKLFEYFKEKAFPYLSTKHLGVFPDHVIDMFINGYRLSLTNHGPTPVKEGSKDPRGGIF
DAVFTRGDQSKLLTSRHDFKEKNLKEISDLIKKELTEGKALGLSHTYANVRINHVINLW
GADFDSNGNLKAIYVTDSDSNASIGMKKYFVGVNSAGKVAISAKEIKEDNIGAQVLGLF
TLSTGQDSWNQTNHHHHHH
SEQ ID NO: 98-Ab62 anti-PECAM Fab V_{H}-C_{H1}-IdeS fusion (nucleic acid)
Caggtgcagcttcaacaaagcggagcagagctgatgaagccaggcgctagtgtgaagatctcatgcaaggccaccggctacacgttcag
ccactattggatcgagtgggtgaaacagaggcccggtcatggcctggagtggatcggcgagatcctgcccgtgaccggctccaccaagta
caccgagaagttcaaggacaaggctaccttcaccgccgacaccttcagcaataccgcctacatgcaactgacaagcctgaccagcgagg
acagcgccgtgtactactgtgccagggaccctcactacagcggcagcacctacgacgccatggactactggggccagggcaccagcgta
acagtgagcgccgctagcaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggctgt
cctacagtcctcaggactctactccctcagcagcgtggtgaccgtgccctccagcagcttgggcacccagacctacatctgcaacgtgaatc
acaagcccagcaacaccaaggtggacaagaaagttgagcccaaatcttgtgcggccgctggaggttcagacagcttttccgccaaccaag
aaataaggtattccgaggtgacgccttatcatgttacgagtgtgtggaccaagggcgttacacccccggcgaactttacgcagggagagga
cgtgttccacgcgccctatgtcgctaatcaaggttggtacgacataactaagacgtttaatggtaaagatgacttgctttgtggcgcagccac
ggcaggaaacatgctgcattggtggttcgatcaaaacaaggatcaaattaagcgctaccttgaggagcatcctgagaaacaaaagatcaactt
caatggggaacagatgttcgatgttaaagaggcgatagataccaaaaatcatcaactcgactccaaactcttcgagtattttaaggagaaggc
gttcccgtatctgtcaacaaagcacttgggggtcttcccggatcatgttattgatatgtttatcaatggatataggctctccctgacaaacca
tggccccaccccagtaaaggaaggctccaaagatccacggggcggcatttttgatgccgtttttacgcgaggggaccaatcaaaactcctgac
cagccggcatgacttcaaagaaaaaaacttgaaagagataagtgacttgataaagaaagagctcaccgaagggaaggctttgggactctctc
acacttacgcaaacgtgcgcatcaatcatgtaatcaatctgtggggggccgatttcgatagcaatggaaatcttaaggcgatctatgtaacgg
attccgattctaatgcgagcattggtatgaaaaagtatttcgtgggagttaactcagctggtaaggtcgctatttccgcaaaagagatcaagg
aggacaacattggtgcgcaagttttgggtctctttacgctcagcacaggccaagattcctggaatcagacgaatcatcatcaccatcaccat
SEQ ID NO: 99-M2.139 anti-collagen type II Fab V_{H}-C_{H1} (amino acid sequence)
EVQLQQSGAELAKPGTSVKMSCKASGYTFISYWMNWVKQRPGQGLEWIGAINPSDGYT
EYNQKFKDKAIMTADRSSSTAYMQLSSLTSEDSALYYCARYGGYFDYWGQGTTLTVSS
AASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC
SEQ ID NO: 100-M2.139 anti-collagen type II Fab V_{H}-C_{H1} (nucleic acid sequence)
gaagtgcagctccaacaatccggtgccgaacttgccaaacccggaacctcagtgaagatgtcctgtaaggcttctgggtatactttcatttcc
tattggatgaactgggtgaagcaacgcccaggacaaggcttggaatggatcggtgcaataaatccctccgacggttataccgaatacaacca
gaaattcaaggacaaagcaatcatgacggctgaccgctccagtagcaccgcgtatatgcagctttccagtcttacgagtgaagactctgcgc
tgtattactgcgctaggtatggaggctacttcgattattggggccagggcacaacgttgacagtcagcagcgcagctagcaccaagggccc
atcggtcttccccctggcaccctcctccaagagcacctctgggggcacagcggccctgggctgcctggtcaaggactacttccccgaacc
ggtgacggtgtcgtggaactcaggcgccctgaccagcggcgtgcacaccttcccggctgtcctacagtcctcaggactctactccctcagc
agcgtggtgaccgtgccctccagcagcttgggcacccagacctacatctgcaacgtgaatcacaagcccagcaacaccaaggggacaa
gaaagttgagcccaaatcttgt
SEQ ID NO: 101-M2.139 anti-collagen type II Fab V_{H} CDR1
SYWMN
SEQ ID NO: 102-M2.139 anti-collagen type II Fab V_{H} CDR2
AINPSDGYTEYNQKFKD
SEQ ID NO: 103-M2.139 anti-collagen type II Fab V_{H} CDR3
YGGYFDY
SEQ ID NO: 104-M2.139 anti-collagen type II Fab V_{L}-C_{L} (amino acid sequence)
DIVLTQSPASLAVSLGQRATISCRASESVEYFGTSLMQWYQQKPGQPPKLLIYAASNVES
GVPARFSGSGSGTDFSLNIHPVEEDDIAMYFCQQSREVPYTFGGGSKLEIKRRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 105-M2.139 anti-collagen type II Fab V_{L}-C_{L} (nucleic acid sequence)
gacatcgtgttgactcaatcacccgctagccttgccgtaagtttgggtcagcgggccacgatcagttgtcgcgccagtgagtcagtcgaata
cttcgggacatcattgatgcaatggtatcaacagaagccgggacagccgcccaaattgcttatctatgccgcgtccaatgtcgaaagtggag
taccggcacgcttctctggaagcggtagcggtactgactttagcttgaatatccatcccgtggaagaagatgacattgctatgtacttctgcc
agcagtccagagaggtcccttatacgttcggtggtggaagcaaattggagattaagcgccgtacggtggctgcaccatctgtcttcatcttcc
cgccatctgatgagcagttgaaatctggaactgcctctgttgtgtgcctgctgaataacttctatcccagagaggccaaagtacagtggaagg
tggataacgccctccaatcgggtaactcccaggagagtgtcacagagcaggacagcaaggacagcacctacagcctcagcagcaccctg
acgctgagcaaagcagactacgagaaacacaaagtctacgcctgcgaagtcacccatcagggcctgagctcgcccgtcacaaagagctt
caacaggggagagtgt
SEQ ID NO: 106-M2.139 anti-collagen type II Fab V_{L} CDR1
RASESVEYFGTSLMQ
SEQ ID NO: 107-M2.139 anti-collagen type II Fab V_{L} CDR2
AASNVES
SEQ ID NO: 108-M2.139 anti-collagen type II Fab V_{L} CDR3
QQSREVPYT
SEQ ID NO: 109-M2.139 anti-collagen type II Fab V_{H}-C_{H1}-IdeS fusion (amino acid)
EVQLQQSGAELAKPGTSVKMSCKASGYTFISYWMNWVKQRPGQGLEWIGAINPSDGYT
EYNQKFKDKAIMTADRSSSTAYMQLSSLTSEDSALYYCARYGGYFDYWGQGTTLTVSS
AASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCAAAGGSDSFSANQEI
RYSEVTPYHVTSVWTKGVTPPANFTQGEDVFHAPYVANQGWYDITKTFNGKDDLLCG
AATAGNMLHWWFDQNKDQIKRYLEEHPEKQKINFNGEQMFDVKEAIDTKNHQLDSKL
FEYFKEKAFPYLSTKHLGVFPDHVIDMFINGYRLSLTNHGPTPVKEGSKDPRGGIFDAVF
TRGDQSKLLTSRHDFKEKNLKEISDLIKKELTEGKALGLSHTYANVRINHVINLWGADF
DSNGNLKAIYVTDSDSNASIGMKKYFVGVNSAGKVAISAKEIKEDNIGAQVLGLFTLST
GQDSWNQTNHHHHHH
SEQ ID NO: 110-M2.139 anti-collagen type II Fab V_{H}-C_{H1}-IdeS fusion (nucleic acid)
gaagtgcagctccaacaatccggtgccgaacttgccaaacccggaacctcagtgaagatgtcctgtaaggcttctgggtatactttcatttcc
tattggatgaactgggtgaagcaacgcccaggacaaggcttggaatggatcggtgcaataaatccctccgacggttataccgaatacaacca
gaaattcaaggacaaagcaatcatgacggctgaccgctccagtagcaccgcgtatatgcagctttccagtcttacgagtgaagactctgcgc
tgtattactgcgctaggtatggaggctacttcgattattggggccagggcacaacgttgacagtcagcagcgcagctagcaccaagggccc
atcggtcttccccctggcaccctcctccaagagcacctctgggggcacagcggccctgggctgcctggtcaaggactacttccccgaacc
ggtgacggtgtcgtggaactcaggcgccctgaccagcggcgtgcacaccttcccggctgtcctacagtcctcaggactctactccctcagc
agcgtggtgaccgtgccctccagcagcttgggcacccagacctacatctgcaacgtgaatcacaagcccagcaacaccaaggggacaa
gaaagttgagcccaaatcttgtgcggccgctggaggttcagacagcttttccgccaaccaagaaataaggtattccgaggtgacgccttatc
atgttacgagtgtgtggaccaagggcgttacacccccggcgaactttacgcagggagaggacgtgttccacgcgccctatgtcgctaatca
aggttggtacgacataactaagacgtttaatggtaaagatgacttgctttgtggcgcagccacggcaggaaacatgctgcattggtggttcga
tcaaaacaaggatcaaattaagcgctaccttgaggagcatcctgagaaacaaaagatcaacttcaatggggaacagatgttcgatgttaaag
aggcgatagataccaaaaatcatcaactcgactccaaactcttcgagtattttaaggagaaggcgttcccgtatctgtcaacaaagcacttgg
gggtcttcccggatcatgttattgatatgtttatcaatggatataggctctccctgacaaaccatggccccaccccagtaaaggaaggctcca
aagatccacggggcggcatttttgatgccgtttttacgcgaggggaccaatcaaaactcctgaccagccggcatgacttcaaagaaaaaaact
tgaaagagataagtgacttgataaagaaagagctcaccgaagggaaggctttgggactctctcacacttacgcaaacgtgcgcatcaatcat
gtaatcaatctgtggggggccgatttcgatagcaatggaaatcttaaggcgatctatgtaacggattccgattctaatgcgagcattggtatg
aaaaagtatttcgtgggagttaactcagctggtaaggtcgctatttccgcaaaagagatcaaggaggacaacattggtgcgcaagttttgggt
ctctttacgctcagcacaggccaagattcctggaatcagacgaatcatcatcaccatcaccat
SEQ ID NO: 111-R6.5 anti-ICAM-1 Fab V_{H}-C_{H1} (amino acid sequence)
QVQLQQSGPELVRPGVSVKISCKGSGYTFIDYAIHWVKESHAKSLEWIGVISAYSGDTN
YNQKFKGKATMTVDKSSNTAYLELARLTSEDSAIYYCARGGWLLLSFDYWGQGTTLTV
SSAASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSC
SEQ ID NO: 112-R6.5 anti-ICAM-1 Fab V_{H}-C_{H1} (nucleic acid sequence)
Caggtccaactgcagcagtcagggcctgagctggtgaggcctggggtctcagtgaagatttcctgcaagggttccggctacacattcattg
attatgctatacactgggtgaaggagagtcatgcaaagagtctagagtggattggagttattagtgcttactctggtgacacaaactacaacc
agaagtttaagggcaaggccacaatgactgttgacaaatcctccaacacagcctatttggaacttgccagattgacatctgaggattctgcca
tctattactgtgcaagagggggatggttactactctcctttgactactggggccaaggcaccactctcacagtctcctcggccgctagcacca
agggcccatcggtcttccccctggcaccctcctccaagagcacctctgggggcacagcggccctgggctgcctggtcaaggactacttccc
cgaaccggtgacggtgtcgtggaactcaggcgccctgaccagcggcgtgcacaccttcccggctgtcctacagtcctcaggactctactcc
ctcagcagcgtggtgaccgtgccctccagcagcttgggcacccagacctacatctgcaacgtgaatcacaagcccagcaacaccaaggt
ggacaagaaagttgagcccaaatcttgt
SEQ ID NO: 113-R6.5 anti-ICAM-1 Fab V_{H} CDR1
DYAIH
SEQ ID NO: 114-R6.5 anti-ICAM-1 Fab V_{H} CDR2
VISAYSGDTNYNQKFKG
SEQ ID NO: 115-R6.5 anti-ICAM-1 Fab V_{H} CDR3
GGWLLLSFDY
SEQ ID NO: 116-R6.5 anti-ICAM-1 Fab V_{L}-C_{L} (amino acid sequence)
DIVMTQSPLSLPVSLGDQASISCRSSQSLVHSNGNNYLHWYLQKSGQAPKLLIYKVSNRF
SGVPDRFSGSGSGTDFTLKISRVEAEDLGVYFCSQSTHVPLTFGGGTKVELRARTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 117-R6.5 anti-ICAM-1 Fab V_{L}-C_{L} (nucleic acid sequence)
gatatcgtgatgacccaaagtccactctccctgcctgtcagtcttggagatcaagcctccatctcttgcagatcaagtcagagccttgtacac
agcaatggaaataactatttacattggtacctgcagaagtcaggccaggctccaaagctcctgatctacaaagtttccaaccgattttctggg
gtcccagacaggttcagtggcagtggatcagggacagatttcacactcaagatcagcagagtggaggctgaggatctgggagtttatttctgc
tctcaaagtacacatgttcctctcacgttcggtggaggcacaaaggtcgaattgagggcacgtacggtggctgcaccatctgtcttcatcttc
ccgccatctgatgagcagttgaaatctggaactgcctctgttgtgtgcctgctgaataacttctatcccagagaggccaaagtacagtggaag
gtggataacgccctccaatcgggtaactcccaggagagtgtcacagagcaggacagcaaggacagcacctacagcctcagcagcaccctg
acgctgagcaaagcagactacgagaaacacaaagtctacgcctgcgaagtcacccatcagggcctgagctcgcccgtcacaaagagctt
caacaggggagagtgt
SEQ ID NO: 118-R6.5 anti-ICAM-1 Fab V_{L} CDR1
RSSQSLVHSNGNNYLH
SEQ ID NO: 119-R6.5 anti-ICAM-1 Fab V_{L} CDR2
KVSNRFS
SEQ ID NO: 120-R6.5 anti-ICAM-1 Fab V_{L} CDR3
SQSTHVPLT
SEQ ID NO: 121-R6.5 anti-ICAM-1 Fab V_{H}-C_{H1}-IdeS fusion (amino acid)
QVQLQQSGPELVRPGVSVKISCKGSGYTFIDYAIHWVKESHAKSLEWIGVISAYSGDTN
YNQKFKGKATMTVDKSSNTAYLELARLTSEDSAIYYCARGGWLLLSFDYWGQGTTLTV
SSAASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCAAAGGSDSFSANQ
EIRYSEVTPYHVTSVWTKGVTPPANFTQGEDVFHAPYVANQGWYDITKTFNGKDDLLC
GAATAGNMLHWWFDQNKDQIKRYLEEHPEKQKINFNGEQMFDVKEAIDTKNHQLDSK
LFEYFKEKAFPYLSTKHLGVFPDHVIDMFINGYRLSLTNHGPTPVKEGSKDPRGGIFDAV
FTRGDQSKLLTSRHDFKEKNLKEISDLIKKELTEGKALGLSHTYANVRINHVINLWGADF
DSNGNLKAIYVTDSDSNASIGMKKYFVGVNSAGKVAISAKEIKEDNIGAQVLGLFTLST
GQDSWNQTNHHHHHH
SEQ ID NO: 122-R6.5 anti-ICAM-1 Fab V_{H}-C_{H1}-IdeS fusion (nucleic acid)
caggtccaactgcagcagtcagggcctgagctggtgaggcctggggtctcagtgaagatttcctgcaagggttccggctacacattcattga
ttatgctatacactgggtgaaggagagtcatgcaaagagtctagagtggattggagttattagtgcttactctggtgacacaaactacaacca
gaagtttaagggcaaggccacaatgactgttgacaaatcctccaacacagcctatttggaacttgccagattgacatctgaggattctgccat
ctattactgtgcaagagggggatggttactactctcctttgactactggggccaaggcaccactctcacagtctcctcggccgctagcaccaa
gggcccatcggtcttccccctggcaccctcctccaagagcacctctgggggcacagcggccctgggctgcctggtcaaggactacttcccc
gaaccggtgacggtgtcgtggaactcaggcgccctgaccagcggcgtgcacaccttcccggctgtcctacagtcctcaggactctactccc
tcagcagcgtggtgaccgtgccctccagcagcttgggcacccagacctacatctgcaacgtgaatcacaagcccagcaacaccaaggtg
gacaagaaagttgagcccaaatcttgtgcggccgctggaggttcagacagcttttccgccaaccaagaaataaggtattccgaggtgacgc
cttatcatgttacgagtgtgtggaccaagggcgttacacccccggcgaactttacgcagggagaggacgtgttccacgcgccctatgtcgct
aatcaaggttggtacgacataactaagacgtttaatggtaaagatgacttgctttgtggcgcagccacggcaggaaacatgctgcattggtgg
ttcgatcaaaacaaggatcaaattaagcgctaccttgaggagcatcctgagaaacaaaagatcaacttcaatggggaacagatgttcgatgtt
aaagaggcgatagataccaaaaatcatcaactcgactccaaactcttcgagtattttaaggagaaggcgttcccgtatctgtcaacaaagcac
ttgggggtcttcccggatcatgttattgatatgtttatcaatggatataggctctccctgacaaaccatggccccaccccagtaaaggaaggc
tccaaagatccacggggcggcatttttgatgccgtttttacgcgaggggaccaatcaaaactcctgaccagccggcatgacttcaaagaaaa
aaacttgaaagagataagtgacttgataaagaaagagctcaccgaagggaaggctttgggactctctcacacttacgcaaacgtgcgcatc
aatcatgtaatcaatctgtggggggccgatttcgatagcaatggaaatcttaaggcgatctatgtaacggattccgattctaatgcgagcatt
ggtatgaaaaagtatttcgtgggagttaactcagctggtaaggtcgctatttccgcaaaagagatcaaggaggacaacattggtgcgcaagtt
ttgggtctctttacgctcagcacaggccaagattcctggaatcagacgaatcatcatcaccatcaccat

1. A composition comprising an immunoglobulin-G degrading enzyme fused to a targeting moiety capable of specifically binding to a cell surface marker.

2. The composition of claim 1, wherein the immunoglobulin-G degrading enzyme is an immunoglobulin-G degrading enzyme of S. pyogenes (IdeS) polypeptide.

3. The composition of claim 2, wherein the IdeS polypeptide has at least 70% sequence identity to SEQ ID NO: 1 and is capable of cleaving human IgG.

4. The composition of claim 1, wherein the targeting moiety is an antibody fragment.

5. The composition of claim 4, wherein the antibody fragment is an scFv or Fab.

6. The composition of claim 1, wherein the targeting moiety is capable of binding to an erythrocyte surface marker.

7. The composition of claim 6, wherein the targeting moiety is capable of binding human glycophorin A.

8. The composition of claim 7, wherein the targeting moiety is an antibody fragment derived from a YTH 89.1 monoclonal antibody.

9. The composition of claim 8, wherein the targeting moiety comprises a polypeptide having CDRs having at least 70% sequence identity with SEQ ID NOS: 4-6.

10. The composition of claim 9, wherein the targeting moiety comprises a polypeptide comprises at least 70% sequence identity with SEQ ID NO: 2.

12. The composition of claim 8, wherein the targeting moiety comprises a polypeptide having CDRs having at least 70% sequence identity with SEQ ID NOS: 9-11.

13. The composition of claim 12, wherein the targeting moiety comprises a polypeptide comprises at least 70% sequence identity with SEQ ID NO: 7.

14. The composition of claim 8, wherein the targeting moiety comprises a heavy chain variable region with at least 70% sequence identity with SEQ ID NO: 2 and a light chain variable region with at least 70% sequence identity with SEQ ID NO: 7.

15. The composition of claim 8, wherein the targeting moiety comprises a first variable region comprising a CDR1 of SEQ ID NO: 4, a CDR2 of SEQ ID NO: 5 and CDR3 of SEQ ID NO: 6 and a second variable region comprising a CDR1 of SEQ ID NO: 9, a CDR2 of SEQ ID NO: 10 and CDR3 of SEQ ID NO: 11.

16. The composition of claim 6, wherein the targeting moiety is capable of binding human Wr^{b} antigen.

17. The composition of claim 16, wherein the targeting moiety is an antibody fragment derived from a Wr^{b} monoclonal antibody.

18. The composition of claim 17, wherein the targeting moiety comprises a polypeptide having CDRs having at least 70% sequence identity with SEQ ID NOS: 16-18.

19. The composition of claim 18, wherein the targeting moiety comprises a polypeptide comprises at least 70% sequence identity with SEQ ID NO: 14.

20. The composition of claim 17, wherein the targeting moiety comprises a polypeptide having CDRs having at least 70% sequence identity with SEQ ID NOS: 21-23.

21. The composition of claim 20, wherein the targeting moiety comprises a polypeptide comprises at least 70% sequence identity with SEQ ID NO: 19.

22. The composition of claim 17, wherein the targeting moiety comprises a heavy chain variable region with at least 70% sequence identity with SEQ ID NO: 14 and a light chain variable region with at least 70% sequence identity with SEQ ID NO: 19.

23. The composition of claim 17, wherein the targeting moiety comprises a first variable region comprising a CDR1 of SEQ ID NO: 16, a CDR2 of SEQ ID NO: 17 and CDR3 of SEQ ID NO: 18 and a second variable region comprising a CDR1 of SEQ ID NO: 21, a CDR2 of SEQ ID NO: 22 and CDR3 of SEQ ID NO: 23.

24. The composition of claim 6, wherein the targeting moiety is capable of binding human Rh17 antigen.

25. The composition of claim 24, wherein the targeting moiety is an antibody fragment derived from a Rh17 monoclonal antibody.

26. The composition of claim 25, wherein the targeting moiety comprises a polypeptide having CDRs having at least 70% sequence identity with SEQ ID NOS: 28-30.

27. The composition of claim 24, wherein the targeting moiety comprises a polypeptide comprises at least 70% sequence identity with SEQ ID NO: 26.

28. The composition of claim 23, wherein the targeting moiety comprises a polypeptide having CDRs having at least 70% sequence identity with SEQ ID NOS: 33-35.

29. The composition of claim 28, wherein the targeting moiety comprises a polypeptide comprises at least 70% sequence identity with SEQ ID NO: 31.

30. The composition of claim 23, wherein the targeting moiety comprises a heavy chain variable region with at least 70% sequence identity with SEQ ID NO: 26 and a light chain variable region with at least 70% sequence identity with SEQ ID NO: 31.

31. The composition of claim 23, wherein the targeting moiety comprises a first variable region comprising a CDR1 of SEQ ID NO: 28, a CDR2 of SEQ ID NO: 29 and CDR3 of SEQ ID NO: 30 and a second variable region comprising a CDR1 of SEQ ID NO: 33, a CDR2 of SEQ ID NO: 34 and CDR3 of SEQ ID NO: 35.

32. The composition of claim 1, wherein the targeting moiety is capable of binding to a platelet surface marker.

33. The composition of claim 32, wherein the targeting moiety is capable of binding human FcγRIIA.

34. The composition of claim 33, wherein the targeting moiety is an antibody fragment derived from an IV.3 monoclonal antibody.

35. The composition of claim 33, wherein the targeting moiety comprises a polypeptide having comprises a first variable region comprising a CDR1 of SEQ ID NO: 65, a CDR2 of SEQ ID NO: 66 and CDR3 of SEQ ID NO: 67 and a second variable region comprising a CDR1 of SEQ ID NO: 68, a CDR2 of SEQ ID NO: 69 and CDR3 of SEQ ID NO: 70.

36. The composition of claim 33, wherein the targeting moiety comprises a polypeptide comprises at least 70% sequence identity with one of SEQ ID NOS: 62-64.

37. The composition of claim 1, wherein the targeting moiety is capable of binding to an endothelial cell surface marker.

38. The composition of claim 37, wherein the targeting moiety is capable of binding human PECAM.

39. The composition of claim 38, wherein the targeting moiety is an antibody fragment derived from a Ab37 monoclonal antibody.

40. The composition of claim 39, wherein the targeting moiety comprises a polypeptide having CDRs having at least 70% sequence identity with SEQ ID NOS: 77-79.

41. The composition of claim 40, wherein the targeting moiety comprises a polypeptide comprises at least 70% sequence identity with SEQ ID NO: 75.

42. The composition of claim 39, wherein the targeting moiety comprises a polypeptide having CDRs having at least 70% sequence identity with SEQ ID NOS: 82-84.

43. The composition of claim 42, wherein the targeting moiety comprises a polypeptide comprises at least 70% sequence identity with SEQ ID NO: 80.

44. The composition of claim 39, wherein the targeting moiety comprises a heavy chain variable region with at least 70% sequence identity with SEQ ID NO: 75 and a light chain variable region with at least 70% sequence identity with SEQ ID NO: 80.

45. The composition of claim 39, wherein the targeting moiety comprises a polypeptide having comprises a first variable region comprising a CDR1 of SEQ ID NO: 77, a CDR2 of SEQ ID NO: 78 and CDR3 of SEQ ID NO: 79 and a second variable region comprising a CDR1 of SEQ ID NO: 82, a CDR2 of SEQ ID NO: 83 and CDR3 of SEQ ID NO: 84.

46. The composition of claim 38, wherein the targeting moiety is an antibody fragment derived from a Ab62 monoclonal antibody.

47. The composition of claim 46, wherein the targeting moiety comprises a polypeptide having CDRs having at least 70% sequence identity with SEQ ID NOS: 89-91.

48. The composition of claim 47, wherein the targeting moiety comprises a polypeptide comprises at least 70% sequence identity with SEQ ID NO: 87.

49. The composition of claim 46, wherein the targeting moiety comprises a polypeptide having CDRs having at least 70% sequence identity with SEQ ID NOS: 94-96.

50. The composition of claim 49, wherein the targeting moiety comprises a polypeptide comprises at least 70% sequence identity with SEQ ID NO: 92.

51. The composition of claim 46, wherein the targeting moiety comprises a heavy chain variable region with at least 70% sequence identity with SEQ ID NO: 87 and a light chain variable region with at least 70% sequence identity with SEQ ID NO: 92.

52. The composition of claim 46, wherein the targeting moiety comprises a polypeptide having comprises a first variable region comprising a CDR1 of SEQ ID NO: 89, a CDR2 of SEQ ID NO: 90 and CDR3 of SEQ ID NO: 91 and a second variable region comprising a CDR1 of SEQ ID NO: 94, a CDR2 of SEQ ID NO: 95 and CDR3 of SEQ ID NO: 96.

53. The composition of claim 37, wherein the targeting moiety is capable of binding human ICAM-1.

54. The composition of claim 53, wherein the targeting moiety is an antibody fragment derived from a R6.5 monoclonal antibody.

55. The composition of claim 54, wherein the targeting moiety comprises a polypeptide having CDRs having at least 70% sequence identity with SEQ ID NOS: 113-115.

56. The composition of claim 55, wherein the targeting moiety comprises a polypeptide comprises at least 70% sequence identity with SEQ ID NO: 111.

57. The composition of claim 54, wherein the targeting moiety comprises a polypeptide having CDRs having at least 70% sequence identity with SEQ ID NOS: 118-120.

58. The composition of claim 57, wherein the targeting moiety comprises a polypeptide comprises at least 70% sequence identity with SEQ ID NO: 116.

59. The composition of claim 54, wherein the targeting moiety comprises a heavy chain variable region with at least 70% sequence identity with SEQ ID NO: 111 and a light chain variable region with at least 70% sequence identity with SEQ ID NO: 116.

60. The composition of claim 54, wherein the targeting moiety comprises a polypeptide having comprises a first variable region comprising a CDR1 of SEQ ID NO: 113, a CDR2 of SEQ ID NO: 114 and CDR3 of SEQ ID NO: 115 and a second variable region comprising a CDR1 of SEQ ID NO: 118, a CDR2 of SEQ ID NO: 119 and CDR3 of SEQ ID NO: 120.

61. The composition of claim 1, wherein the targeting moiety is capable of binding to a cartilage cell surface marker.

62. The composition of claim 61, wherein the targeting moiety is capable of binding human collagen type II.

63. The composition of claim 62, wherein the targeting moiety is an antibody fragment derived from a M2.139 monoclonal antibody.

64. The composition of claim 63, wherein the targeting moiety comprises a polypeptide having CDRs having at least 70% sequence identity with SEQ ID NOS: 101-103.

65. The composition of claim 64, wherein the targeting moiety comprises a polypeptide comprises at least 70% sequence identity with SEQ ID NO: 99.

66. The composition of claim 63, wherein the targeting moiety comprises a polypeptide having CDRs having at least 70% sequence identity with SEQ ID NOS: 106-108.

67. The composition of claim 66, wherein the targeting moiety comprises a polypeptide comprises at least 70% sequence identity with SEQ ID NO: 104.

68. The composition of claim 63, wherein the targeting moiety comprises a heavy chain variable region with at least 70% sequence identity with SEQ ID NO: 99 and a light chain variable region with at least 70% sequence identity with SEQ ID NO: 104.

69. The composition of claim 63, wherein the targeting moiety comprises a polypeptide having comprises a first variable region comprising a CDR1 of SEQ ID NO: 101, a CDR2 of SEQ ID NO: 102 and CDR3 of SEQ ID NO: 103 and a second variable region comprising a CDR1 of SEQ ID NO: 106, a CDR2 of SEQ ID NO: 107 and CDR3 of SEQ ID NO: 108.

70. A method of degrading pathogenic IgG on cells comprising contacting the cells with a composition of one of claims 1-69.

71. The method of claim 70, wherein contacting the cells with the composition prevents or reduces destruction of the cells by the cell-bound IgG.

72. A method of treating a disease or condition mediated by pathogenic IgG binding to red blood cells comprising administering a composition of one of claims 1-31 to a subject in need thereof.

73. The method of claim 72, wherein the subject suffers from or is at risk of autoimmune hemolytic anemia (wAIHA), IgG-mediated hemolytic transfusion reaction (HTR), or hemolytic disease of the fetus and newborn (HDFN).

74. A method of treating a disease or condition mediated by pathogenic IgG binding to platelets comprising administering a composition of one of claim 1-5 or 32-36 to a subject in need thereof.

75. The method of claim 74, wherein the subject suffers from or is at risk of immune thrombocytopenia (ITP).

76. A method of treating a disease or condition mediated by pathogenic IgG binding to endothelial comprising administering a composition of one of claim 1-5 or 37-60 to a subject in need thereof.

77. The method of claim 76, wherein the subject suffers from or is at risk of immune vasculitis, Goodpasture's (anti-GMB) disease, or organ transplant rejection.

78. A method of preventing organ transplant rejection comprising administering a composition of one of claim 1-5 or 37-60 to an organ to be transplanted, a subject to receive an organ transplant, or a subject following organ transplant.

79. A method of treating a disease or condition mediated by pathogenic IgG binding to cartilage comprising administering a composition of one of claim 1-5 or 61-69 to a subject in need thereof.

80. The method of claim 79, wherein the subject suffers from or is at risk of autoimmune arthritis.

81. The method of claim 80, wherein the autoimmune arthritis is rheumatoid arthritis.

82. The method of one of claims 70-77 and 79-81, wherein the composition is administered intravenously.

83. The method of one of claims 70-77 and 79-82, wherein the administration is followed by a subsequent administration of untargeted IdeS to the subject.
